(19) United States
(12) Patent Application Publication
(10) Pub. No.: US 2017/0368052 A1
(43) Pub. Date: Dec. 28, 2017

(54) CRYSTAL FORMS OF APOMORPHINE AND USES THEREOF

(71) Applicant: NeuroDerm, Ltd., Rehovot (IL)

(72) Inventors: Oron Yacoby-Zeevi, Moshav Bitsaron (IL); Mara Nemas, Gedera (IL); Jonathan Cummins, Didcot (GB); Petra Dieterich, Abingdon (GB)

(21) Appl. No.: 15/538,972

(22) PCT Filed: Dec. 23, 2015
(86) PCT No.: PCT/IL2015/051246

§ 371 Date: Jun. 22, 2017

Related U.S. Application Data
(60) Provisional application No. 62/240,611, filed on Oct. 13, 2015.
(60) Provisional application No. 62/096,352, filed on Dec. 23, 2014.

(51) Int. Cl. A61K 31/473; A61K 47/10; A61K 9/08; A61K 9/00; A61K 31/485; A61K 31/05; C07D 221/18; A61K 47/22

(52) U.S. Cl. CPC A61K 31/473; C07D 221/18; A61K 47/10; A61K 47/22; A61K 9/0019; A61K 31/485; A61K 31/05; A61K 9/08; A61K 2300/00

(57) ABSTRACT

The present invention provides solid crystalline forms of apomorphine free base or a hydrate, solvate, or co-crystals thereof. Such crystalline forms may be advantageous over amorphous forms of apomorphine, e.g., amorphous salt forms such as acid addition salts of apomorphine, because of their increased/greater stability and/or improved pharmacological properties, e.g., decreased adverse reactions at the site of administration. The invention further provides liquid formulations obtained by dissolving said crystalline forms of apomorphine in a solvent, as well as a method for treatment of a neurological or movement disorder, e.g., Parkinson's disease, or a condition associated therewith, by administration of said liquid formulations.

### CROSS-REFERENCE TO RELATED APPLICATIONS

This application claims the benefit of U.S. Provisional Patent Application No. 62/096,352, filed Dec. 23, 2014, and U.S. Provisional Patent Application No. 62/240,611, filed Oct. 13, 2015, the entire content of which being herewith incorporated by reference as if fully disclosed herein.

### TECHNICAL FIELD

The present invention provides solid crystalline forms of apomorphine free base or a hydrate or solvate thereof, as well as a method for the preparation thereof, and liquid formulations obtained by dissolving said crystalline forms of apomorphine in a solvent. Such formulations are useful in the treatment of a neurological or movement disorder, e.g., Parkinson's disease, or a condition associated therewith.

Abbreviations: BHT, butylated hydroxytoluene; DCM, dichloromethane; DMSO, dimethyl sulfoxide; DSC, differential scanning calorimetry; DTA, differential thermal analysis; FTIR, fourier transform infrared spectroscopy; GVS, gravimetric vapour sorption; HPLC, high performance liquid chromatography; HSM, hot stage microscopy; IPA, isopropanol (isopropyl alcohol); IR, infrared spectroscopy; J, Joule; KF, Karl Fischer (determination of the water content by coulometric titration); LC-MS, liquid chromatography-mass spectrometry; MEK, methyl ethyl ketone; MET/CR, chromatography method reference; NMR, nuclear magnetic resonance; pKₐ, −log (Kₐ), acid dissociation constant; PLM, polarized light microscopy; RH, relative humidity (water activity*100); RRT, relative retention time; RT, room temperature (ambient, typically: 18 to 23° C.); STA, simulated thermal analysis (STA=TGA+DTA); TBME, tert-butyl methyl ether; TCNB, 2,3,5,6-tetrachloronitrobenzene; TGA, thermogravimetric analysis; THF, tetrahydrofuran; TMP, 2,2,6,6-tetramethylpiperidine; w/w, weight/weight; XRPD, X-ray powder diffraction.

### BACKGROUND ART

Parkinson's disease is a progressive degenerative disease of the central nervous system. Although the primary cause of Parkinson's disease is not known, it is characterized by the degeneration of dopaminergic neurons of the substantia nigra. The substantia nigra is located in the midbrain and is involved in controlling voluntary movements. The degeneration of neurons causes a shortage of dopamine in the brain, which is believed to cause the observable symptoms of the disease. These symptoms include paucity of movement and rigidity, resting tremor, bradykinesia, and poor balance.

There are a variety of therapeutic treatments available for Parkinson's disease. The best known is levodopa, a dopamine precursor; however, treatment with levodopa can cause serious side-effects, especially over a long term. One such complication of long-term treatment with levodopa is the development of rapid fluctuations in clinical state, where a patient switches suddenly between mobility and immobility for periods ranging from a few minutes to a few hours. This phenomenon is known as the “on-off effect”, the “on” state characterized by the levodopa benefit of early normal motor functioning and the “off” state characterized by akinesia—abrupt loss of mobility, e.g., where a patient may suddenly stop while walking. Approximately half of patients on levodopa therapy will develop such on-off effects after several years of therapy.

While apomorphine hydrochloride has proved effective in treating “off” episodes in patients with Parkinson's disease, a common and serious side effect of administering apomorphine hydrochloride by subcutaneous injection is the development of subcutaneous nodules at the injection site, which can become infected, necessitating treatment or surgical involvement. A majority of people on infused apomorphine develop nodules, and a new nodule may form every time the infusion needle is re-sited, which may happen on a daily basis. Such nodules may be painful, limit available infusion sites and interfere with absorption. Further, unstable compositions (e.g., having precipitate of apomorphine or other agents) may be the cause, or exacerbate, such nodule side effects. Thus, there is a need for new, stable formulations of apomorphine which are safe and effective for administration to patients.

### SUMMARY OF INVENTION

In one aspect, the present invention provides a solid crystalline form of apomorphine free base or a hydrate, solvate, or co-crystal thereof, more particularly, such a solid crystalline form of apomorphine free base or a solvate thereof, e.g., an alcohol solvate crystal of apomorphine free base. In a particular embodiment, the present invention provides a solid crystalline form of apomorphine solvate, wherein the solvate forming solvent is (C₁-C₈)alkanol, preferably IPA, i.e., to a solid crystalline form of apomorphine•IPA.

In another aspect, the present invention provides a liquid formulation obtained by dissolving a solid crystalline form of apomorphine free base or hydrate, solvate, or co-crystal thereof, as disclosed herein, e.g., said solid crystalline form of apomorphine free base or solvate thereof, in a solvent. The liquid formulation of the invention may further comprise an antioxidant. Particular such liquid formulations further comprise one or more pharmaceutically acceptable carriers or excipients, i.e., are pharmaceutically acceptable liquid formulations.

In yet another aspect, the present invention relates to a method of treating a neurological or movement disease or disorder, or a condition associated therewith, in a patient in need thereof, comprising administering to said patient a liquid formulation as disclosed herein. Examples of neurological or movement diseases or disorders include, without being limited to, Parkinson's disease, Alzheimer's disease, and akinesia, and non-limiting examples of conditions associated with neurological or movement diseases or disorders include alcoholism, opiate addiction, and erectile dysfunction.

In still another aspect, the present invention relates to a liquid formulation as disclosed herein, for use in the treatment of a neurological or movement disease or disorder, or a condition associated therewith.

In a further aspect, the present invention relates to a method of producing said solid crystalline form of apomorphine free base or a solvate thereof, said method comprising:
a) dissolving apomorphine hydrochloride and, optionally, an antioxidant such as an ascorbate-based antioxidant, e.g., ascorbic acid-6-palmitate, in a solvent selected from (C₁-C₃)alkyl-, dialkyl-, or trialkylbenzene, pyridine, pyrrole, (C₁-C₃)alkyl-CN, (C₁-C₃)alkyl-NO₂, (R)₂NC(O)H wherein R is H or (C₁-C₆)alkyl, (C₁-C₅)alkylC(O)O— esters, (C₁-C₈)alkanol, (C₂-C₈)alkyl-O—(C₁-C₈)alkyl, (C₃-C₈)cyclic ether, (C₃-C₇)cyclic diether, (C₂-C₆)glycol, or a mixture thereof;
b) contacting the solution obtained in (a) with a base in an amount sufficient to generate the apomorphine free base or solvate thereof; and
c) subjecting the solution to conditions that result in crystallization of the apomorphine free base or solvate thereof, thereby producing said crystalline form of apomorphine free base or solvate thereof.

In particular embodiments, the method of the present invention comprises, in step (a), dissolving apomorphine hydrochloride and, optionally, said antioxidant, in IPA, to thereby obtained, following step (c), a solid crystalline form of apomorphine•IPA.

### BRIEF DESCRIPTION OF THE FIGURES

FIGS. 1A-1F show photo micrographs of samples of apomorphine free base solvates crystallized from various solvents at ×100 magnification under plain- and cross-polarized light (1A-1F refer to samples B, G, H, M, O and P, respectively, in Table 1).

FIG. 2 shows the appearance of samples of apomorphine free base isolated from water, IPA or MeOAc (left, middle and right bottle in each one of the panels) that were left open to air over a period of days.

FIG. 3 shows the appearance of the salt release reaction mixtures during a base screen of organic bases with n-BuOH as the reaction solvent. Bases used (from left to right) were diisopropylamine, piperidine, pyrrolidine, TMP, diethylamine, betaine, L-arginine, lysine, Ca(OH)₂ and Mg(OH)₂.

FIG. 4 shows photomicrographs of apomorphine free base mono-IPA solvate crystallized from a solution of IPA and 3-amino-1-propanol at ×100 magnification under plain- and cross-polarized light.

FIGS. 5A-5B show photomicrographs of apomorphine free base mono-IPA solvate isolated from hot IPA solution using seed crystals (5A) vs. previously isolated material isolated from a reaction run under similar conditions, but with recrystallization at 40-45° C. (5B), at ×100 magnification under plain- and cross-polarized light.

FIG. 6 shows the stabilizing effect of certain antioxidants on apomorphine free base in solution. Reaction A (control) contained no added antioxidant; reaction B contained 10 wt % ascorbic acid-6-palmitate; and reaction C contained 10 wt % BHT.

FIG. 7 shows photographs of reactions with or without antioxidant held at 40-45° C. for extended periods of time to monitor discoloration of the product resulting from air sensitivity.

FIG. 8 shows a comparison of HPLC profiles of product isolated via aqueous-based (sample A0513-126-01) and non-aqueous (sample A0486-178-B1) procedures (top); and micrographs under plain- and cross-polarized light at ×40 magnification of the products of the aqueous-based and non-aqueous procedures (bottom) (nd—not detected).

FIG. 9 shows XRPD data for apomorphine free base IPA solvate isolated from IPA recrystallization following salt release under aqueous conditions (sample A0486-178-B1).

FIG. 10 shows XRPD data for apomorphine free base IPA solvate produced by the non-aqueous route (sample A0513-126-01, lower trace) and isolated from water (sample A0513-132-07, upper trace).

FIG. 11 shows DSC data of sample A0526-010-A1 apomorphine•1*IPA (from recrystallized demonstration batch).

FIG. 12 shows XRPD data for sample A0526-010-A1 before (lower diffractogram) and heated to 140° C. after release of IPA (upper diffractogram).

FIG. 13 shows a photomicrograph of sample A0526-010-A1 heated to 180° C., contents expressed from crucible, recorded under cross polarised light ×100 (note irregular morphology).

FIG. 14 shows XRPD data of sample A0526-010-A1 before (lower diffractogram) and heated to 180° C. after release of IPA (upper diffractogram).

FIG. 15 shows STA(TGA) analysis of sample A0526-010-A1 apomorphine•1*IPA (Form A).

FIG. 16 shows XRPD data of sample A0505-124-C1 apomorphine•1*IPA (Form A) resulting from a rapid cooling experiment.

FIG. 17 shows XRPD data of sample A0505-096-A1 apomorphine•0.9*IPA (upper diffractogram) compared with authentic apomorphine•1.0*IPA Form A (A0526-010-A1, lower diffractogram).

FIG. 18 shows DSC of sample A0505-096-A1 apomorphine•0.9*IPA.

FIG. 19 shows XRPD data of sample A0505-106-A1 after suspension inter-conversion (upper diffractogram), compared to the product isolated after evaporation (A0505-096-A1, lower diffractogram) and to authentic apomorphine•1*IPA, Form A (middle diffractogram).

FIG. 20 shows XRPD data of sample A0505-096-B1.

FIG. 21 shows XRPD data of apomorphine•1.0*TBME before treatment (A0505-080-D1, middle diffractogram), after stirring in IPA at 45-50° C. for 6 days (A0505-116-C1, upper diffractogram), compared to authentic apomorphine•1.0*IPA (Form A) (A0526-010-A1, bottom diffractogram).

FIG. 22 shows GVS water sorption/desorption isotherm for Form A (A0526-010-A1).

FIG. 23 shows ¹H NMR data for sample A0526-010-A1. The spectrum was acquired in CD₃OD and referenced to the solvent residual at 3.31 ppm. The sample contained an internal standard TCNB (1H, s) at 8.1 ppm.

FIG. 24 shows ¹H NMR data for sample A0526-004-B1 of apomorphine•1*IPA isolated from the demonstration batch after crystallization from IPA. The spectrum was acquired in DMSO-d₆ and referenced to the non-deuterated solvent residual at δ=2.50 ppm. No internal standard was present in the sample.

FIG. 25 shows XRPD data for sample A0526-004-B1 of apomorphine•1*IPA isolated from the demonstration batch after crystallization from isopropanol.

FIG. 26 shows a photomicrograph (sample A0526-004-B1) of the isolated apomorphine•1*IPA solvate crystals at ×40 magnification under normal polarised light (left); and a corresponding photomicrograph (also sample A0526-004-B1) of the isolated crystals at ×40 magnification under cross polarised light (right).

FIG. 27 shows ¹H NMR data for sample A0526-010-A1, i.e., apomorphine•1*IPA isolated from the demonstration batch after recrystallization from IPA. The spectrum was acquired in DMSO-d₆ and referenced to the non-deuterated solvent residual at δ=2.50 ppm. No internal standard was present in the sample.

FIG. 28 shows DSC data for sample A0526-010-A1, i.e., apomorphine•1*IPA isolated from the demonstration batch after crystallization from IPA.

FIG. 29 shows XRPD data for sample A0526-010-A1, i.e., apomorphine•1*IPA isolated from the demonstration batch after crystallization from IPA.

FIG. 30 shows HPLC data for sample A0526-010-A1, i.e., the recrystallized demonstration batch.

FIGS. 31A-31B show GVS water sorption/desorption isotherm for apomorphine•1*IPA Form A (A0526-010-A1) (31A); and GVS water sorption/desorption kinetics for apomorphine•IPA Form A (A0505-022-01) (31B).

FIG. 32 shows STA and TGA for apomorphine•1*IPA Form A (A0526-010-A1).

FIG. 33 shows XRPD data for apomorphine formamide solvate, sample A0530-004-F1.

FIG. 34 shows XRPD data for apomorphine acetone solvate, sample A0530-010-F1.

FIG. 35 shows XRPD data for apomorphine TBME solvate, sample A0530-010-G1.

FIG. 36 shows XRPD data for apomorphine methyl acetate solvate, sample A0530-010-H1.

FIG. 37 shows XRPD data for apomorphine THF solvate, sample A0530-010-K1.

FIG. 38 shows XRPD data for apomorphine ethanol solvate, sample A0530-010-O1.

FIG. 39 shows XRPD data for apomorphine acetonitrile solvate, sample A0530-010-Q1.

FIG. 40 shows XRPD data for apomorphine hydrate, sample A0530-010-X1.

FIG. 41 shows XRPD data for apomorphine 1,4-dioxane solvate, sample A0530-010-Z1.

FIG. 42 shows XRPD data for apomorphine nitromethane solvate, sample A0530-010-AB1.

FIG. 43 shows XRPD data for apomorphine pyridine solvate, sample A0530-010-AF1.

FIG. 44 shows XRPD data for apomorphine ethylene glycol solvate, sample A0530-010-AT1.

FIG. 45 shows XRPD data for apomorphine•0.5*acetone solvate, sample A0505-080-A2.

FIG. 46 shows DSC data for apomorphine•0.5*acetone solvate, sample A0505-080-A2.

FIG. 47 shows XRPD data for apomorphine•1.0*TBME solvate, sample A0505-080-D1 (prepared under anhydrous conditions).

FIG. 48 shows XRPD data for apomorphine•1.0*TBME solvate, sample A0505-090-D1 (prepared under aqueous conditions).

FIG. 49 shows DSC data for apomorphine•1.0*TBME solvate, sample A0505-080-D1 (prepared under anhydrous conditions).

FIG. 50 shows XRPD data for apomorphine•0.2*cumene•0.5*IPA solvate, sample A0505-080-E1 (prepared under anhydrous conditions).

FIG. 51 shows XRPD data for apomorphine•0.2*cumene•0.5*IPA solvate, sample A0505-090-E1 (prepared under aqueous conditions).

FIG. 52 shows DSC data for apomorphine•0.2*cumene•0.5*IPA solvate, sample A0505-080-E1 (prepared under anhydrous conditions).

FIG. 53 shows XRPD data for apomorphine•0.5*EtOH solvate, sample A0505-080-G1 (prepared under anhydrous conditions).

FIG. 54 shows XRPD data for apomorphine•0.5*EtOH solvate, sample A0505-090-G1 (prepared under aqueous conditions).

FIG. 55 shows DSC data for apomorphine•0.5*EtOH solvate, sample A0505-080-G1 (prepared under anhydrous conditions).

FIG. 56 shows XRPD data for apomorphine•0.5*THF solvate, sample A0505-080-O2 (prepared under anhydrous conditions).

FIG. 57 shows DSC data for apomorphine•0.5*THF solvate, sample A0505-080-O2.

### DETAILED DESCRIPTION OF THE INVENTION

In one aspect, the present invention provides a solid crystalline form of apomorphine free base or a hydrate, solvate, or co-crystal thereof. In a more particular such aspect, the present invention provides a solid crystalline form of apomorphine free base or a solvate thereof. Such crystalline forms of apomorphine can be advantageous over amorphous form of apomorphine, e.g., amorphous salt forms such as acid addition salts of apomorphine, due to their increased/greater stability and/or improved pharmacological properties, e.g., decreased adverse reactions such as nodule side effects at the site of administration, as compared to the corresponding amorphous forms.

The term “solvate”, with respect to the solid crystalline form of the present invention, refers to a solid crystalline form consisting of apomorphine free base molecules and molecules of one or more solvents each referred to herein as “a solvate forming solvent”. Solid crystalline forms of apomorphine free base solvate comprising molecules of more than one solvent are also referred to herein as “solid crystalline form of apomorphine free base mixed solvate”. As shown herein, such crystalline forms can be prepared by crystallization from a solvent or a mixture of more than one, e.g., two or three, solvents in which the apomorphine free base is dissolved. Yet, in cases wherein crystallization is carried out from a solvent mixture, and depending on the crystallization procedure and conditions, the solid crystalline forms of the apomorphine solvate obtained may comprise molecules of one or more of the solvents present in said solvent mixture.

In certain embodiments, the present invention provides a solid crystalline form of apomorphine solvate as defined above, wherein the solvate forming solvent is selected from a (C₁-C₃)alkyl-, dialkyl-, or trialkylbenzene, pyridine, pyrrole, (C₁-C₃)alkyl-CN, (C₁-C₃)alkyl-NO₂, (R)₂NC(O)H wherein R is H or (C₁-C₆)alkyl, (C₁-C₅)alkylC(O)O— esters such as (C₁-C₅)alkyl-C(O)O—(C₁-C₅)alkyl, straight or branched (C₁-C₈)alkanol, i.e., (C₁-C₈)alcohol, (C₂-C₈)alkyl-O(C₁-C₈)alkyl, (C₃-C₈)cyclic ether, (C₃-C₇)cyclic diether, (C₂-C₆)glycol, or a mixture thereof.

The term “alkyl” as used herein typically means a linear or branched saturated hydrocarbon radical having 1-8 carbon atoms and includes, e.g., methyl, ethyl, n-propyl, isopropyl, n-butyl, sec-butyl, isobutyl, tert-butyl, n-pentyl, 2,2-dimethylpropyl, n-hexyl, n-heptyl, n-octyl, and the like. Preferred are (C₁-C₅)alkyl groups, more preferably (C₁-C₃)alkyl groups, i.e., methyl, ethyl, n-propyl, and isopropyl.

The term “(C₁-C₅)alkylC(O)O— esters” as used herein refers to a molecule wherein the group (C₁-C₅)alkyl-COO— is linked, via the carboxylic group thereof, to a group such as (C₁-C₈)alkyl, (C₂-C₁₂)alkanoyloxymethyl, 1-(alkanoyloxy)ethyl having from 4-9 carbon atoms, 1-methyl-1-(alkanoyloxy)-ethyl having from 5-10 carbon atoms, alkoxycarbonyloxymethyl having from 3-6 carbon atoms, 1-(alkoxycarbonyl-oxy)ethyl having from 4-7 carbon atoms, 1-methyl-1-(alkoxycarbonyloxy)ethyl having from 5-8 carbon atoms, N-(alkoxycarbonyl)aminomethyl having from 3-9 carbon atoms, 1-(N-(alkoxycarbonyl)amino)ethyl having from 4-10 carbon atoms, 3-phthalidyl, 4-crotonolactonyl, gamma-butyrolacton-4-yl, di-N,N—(C₁-C₂) alkylamino(C₂-C₃)alkyl (such as β-dimethylaminoethyl), carbamoyl-(C₁-C₂) alkyl, N,N-di(C₁-C₂)alkylcarbamoyl-(C₁-C₂)alkyl and piperidino-, pyrrolidino- or morpholino(C₂-C₃)alkyl. According to the present invention, particular such molecules are selected from (C₁-C₅)alkyl-C(O)O—(C₁-C₅)alkyl.

The terms “(C₃-C₈)cyclic ether” and (C₃-C₇)cyclic diether, as used herein, refer to a cyclic organic compound having 4-8 carbon atoms and containing an ether group, i.e., a group of the formula R—O—R wherein R each independently represents an alkyl or aryl group, and to a cyclic organic compound having 3-7 carbon atoms and containing two ether groups as defined above. Examples of such compounds include, without being limited to, furan, furfural, THF, dihydrofuran, 2-furan methanol, 2-methyl-tetrahydrofuran, 2,5-dimethyl-tetrahydrofuran, 2-methyl furan, 2-ethyl-tetrahydrofuran, 2-ethyl furan, hydroxymethylfurfural, 3-hydroxytetrahydrofuran, tetrahydro-3-furanol, 2,5-dimethyl furan, 5-hydroxymethyl-2(5H)-furanone, dihydro-5-(hydroxymethyl)-2(3H)-furanone, tetrahydro-2-furoic acid, dihydro-5-(hydroxymethyl)-2(3H)-furanone, tetrahydrofurfuryl alcohol, 1-(2-furyl)ethanol, hydroxymethyltetrahydrofurfural, dioxanes, dioxalanes, pyrans, tetrahydropyrans, dioxins, oxepane, oxypine, and isomers thereof.

The term “glycol” as used herein refers to an organic alcohol having 2-6 carbon atoms, wherein two hydroxyl groups are attached to different carbon atoms of the molecule. Non-limiting examples of glycols include ethylene glycol, propylene glycol, dipropylene glycol, butylene glycol, and the like.

In particular such embodiments, the solvate forming solvent is selected from a formamide, acetone, TBME, THF, acetonitrile, nitromethane, pyridine, ethylene glycol, cumene, methyl acetate (MeOAc), ethyl acetate (EtOAc), isopropyl acetate, methanol (MeOH), ethanol (EtOH), IPA, n-propanol, n-butanol (n-BuOH), 1,4-dioxane, or a mixture thereof such as a mixture of IPA and cumene. More particular such embodiments are those wherein the solid crystalline form comprises about 0.1 to about 1.1, preferably about 0.5 to about 1.0, mol of formamide, acetone, TBME, THF, acetonitrile, nitromethane, pyridine, ethylene glycol, cumene, MeOAc, EtOH, IPA, or 1,4-dioxane per about 1 mol apomorphine free base.

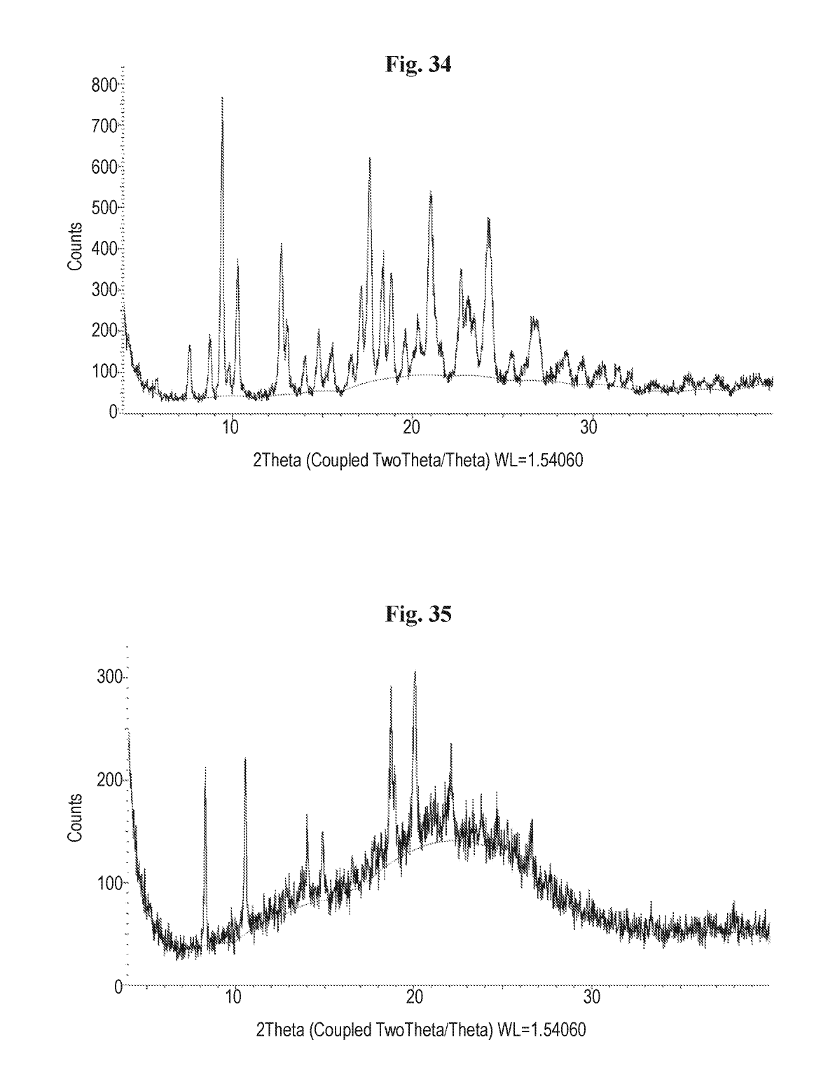

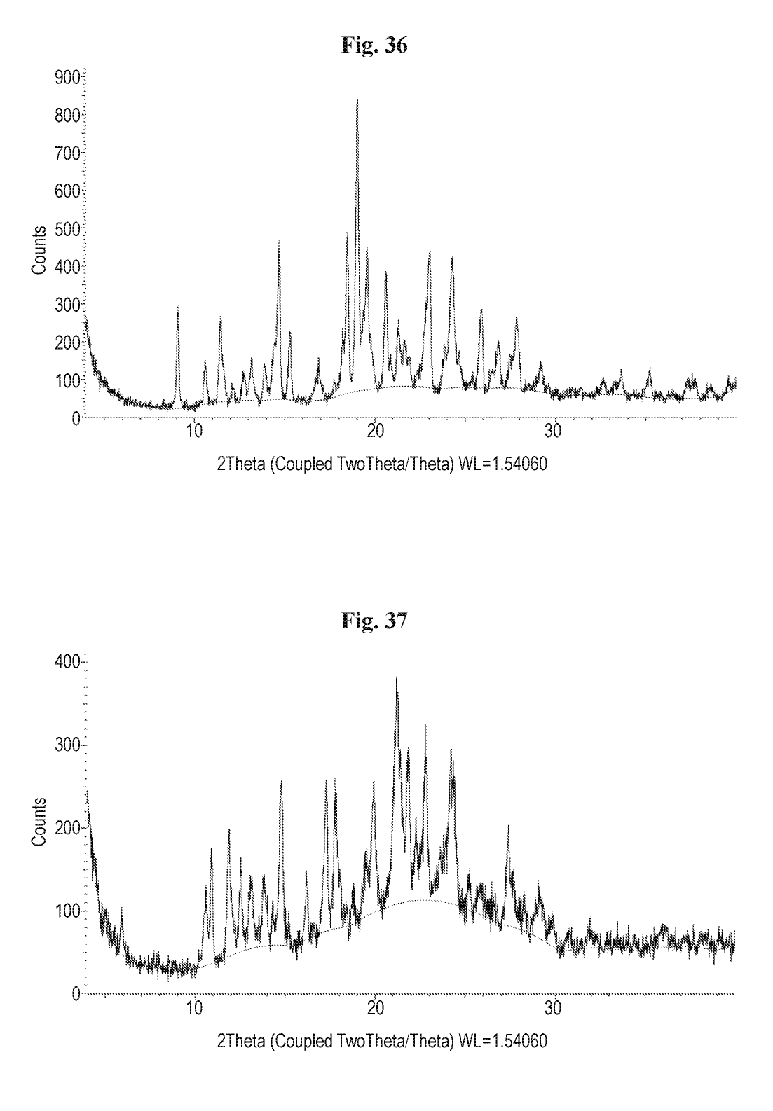

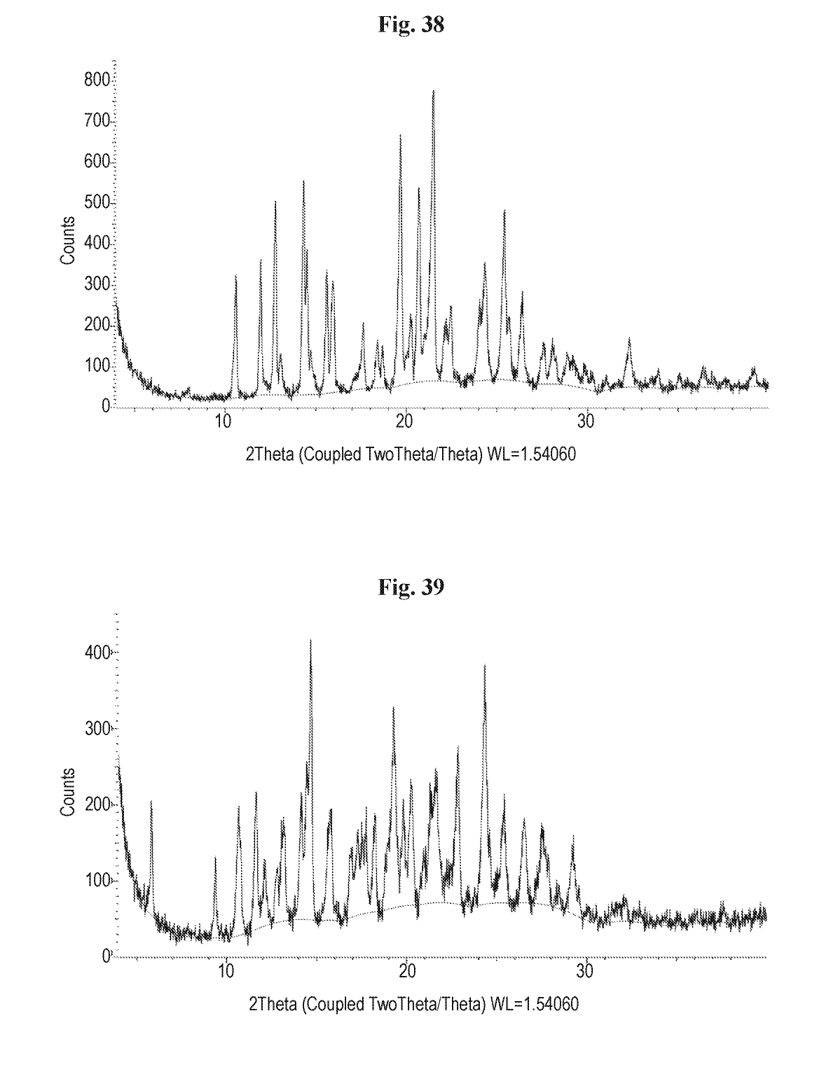

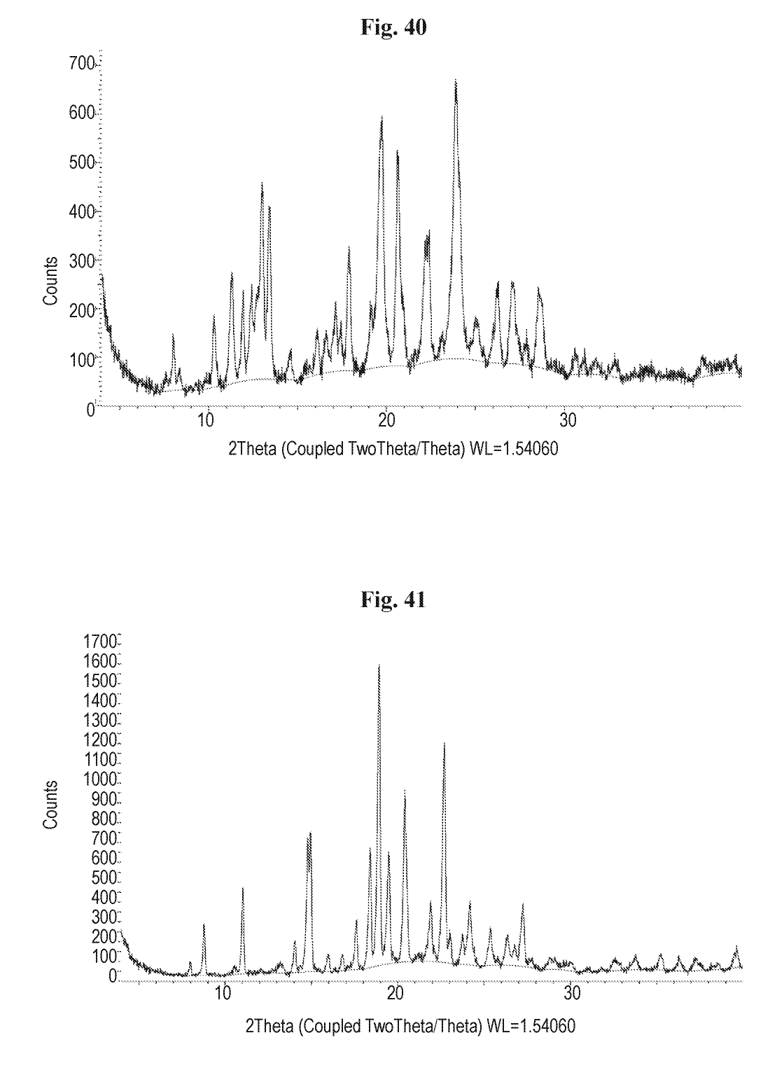

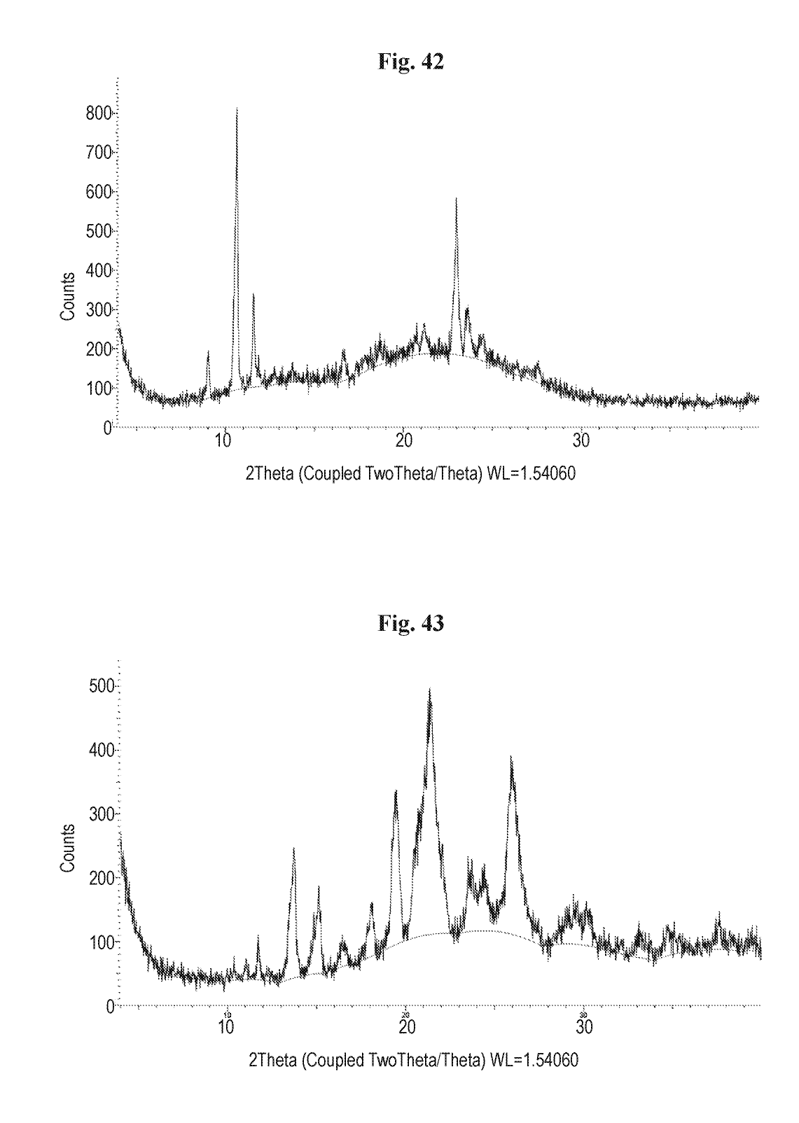

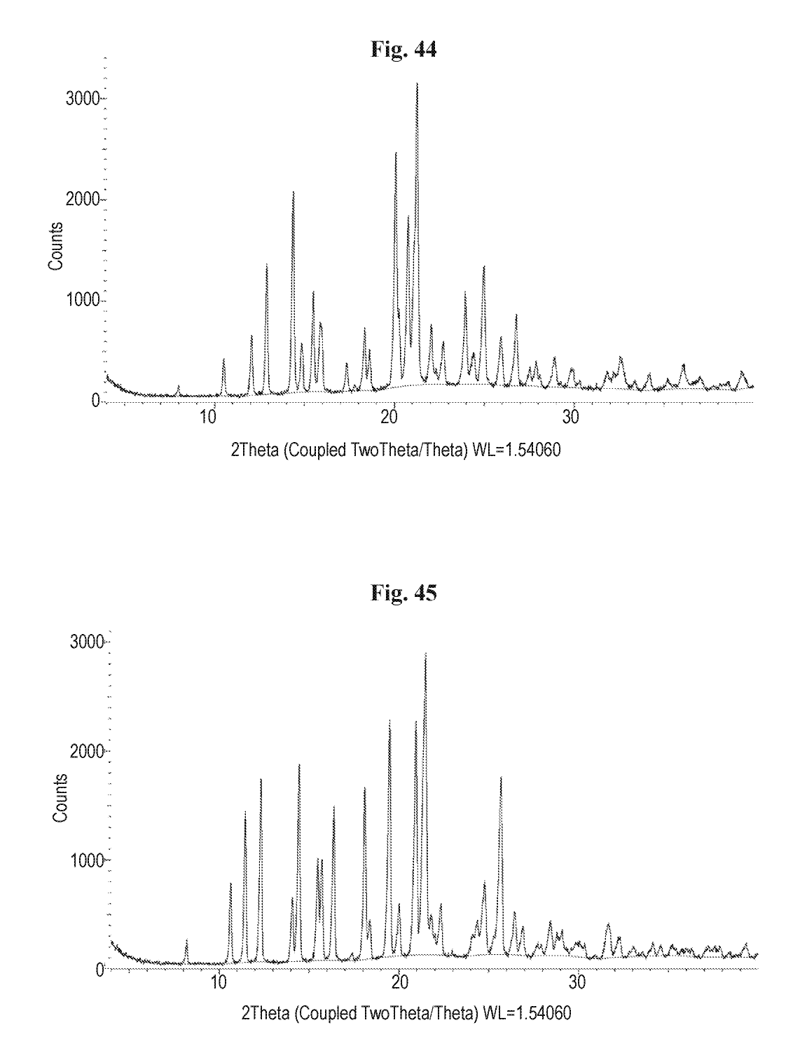

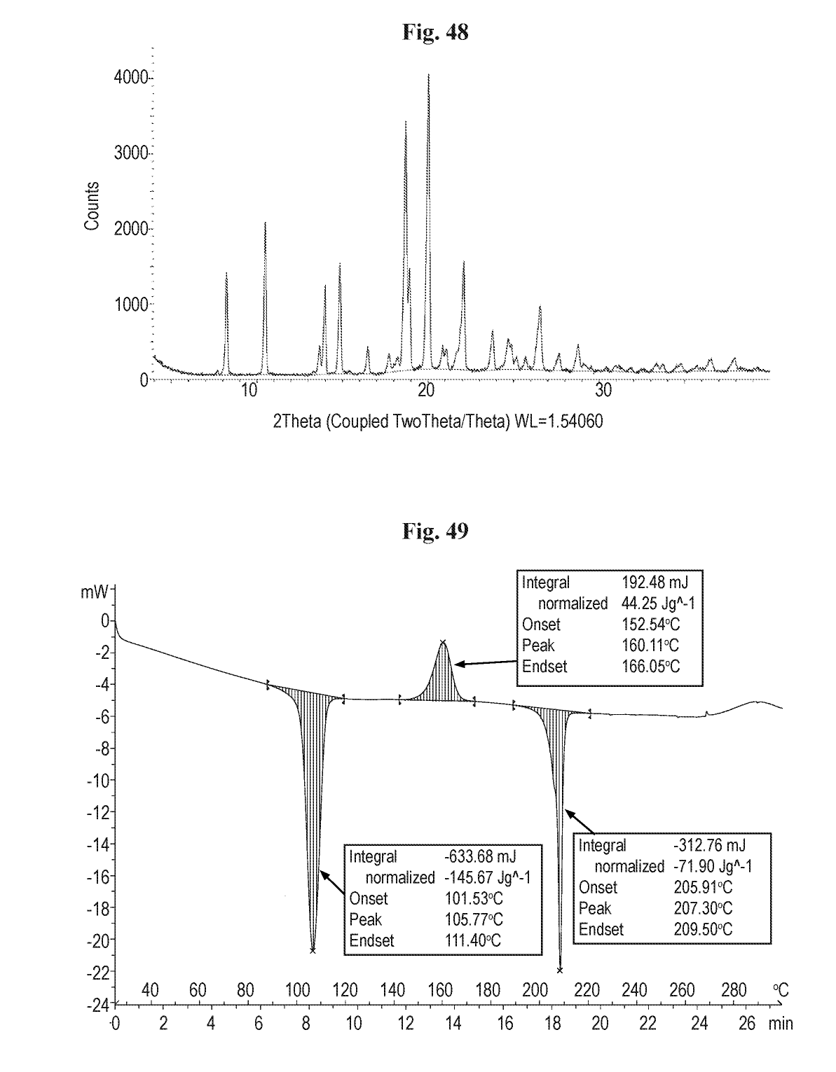

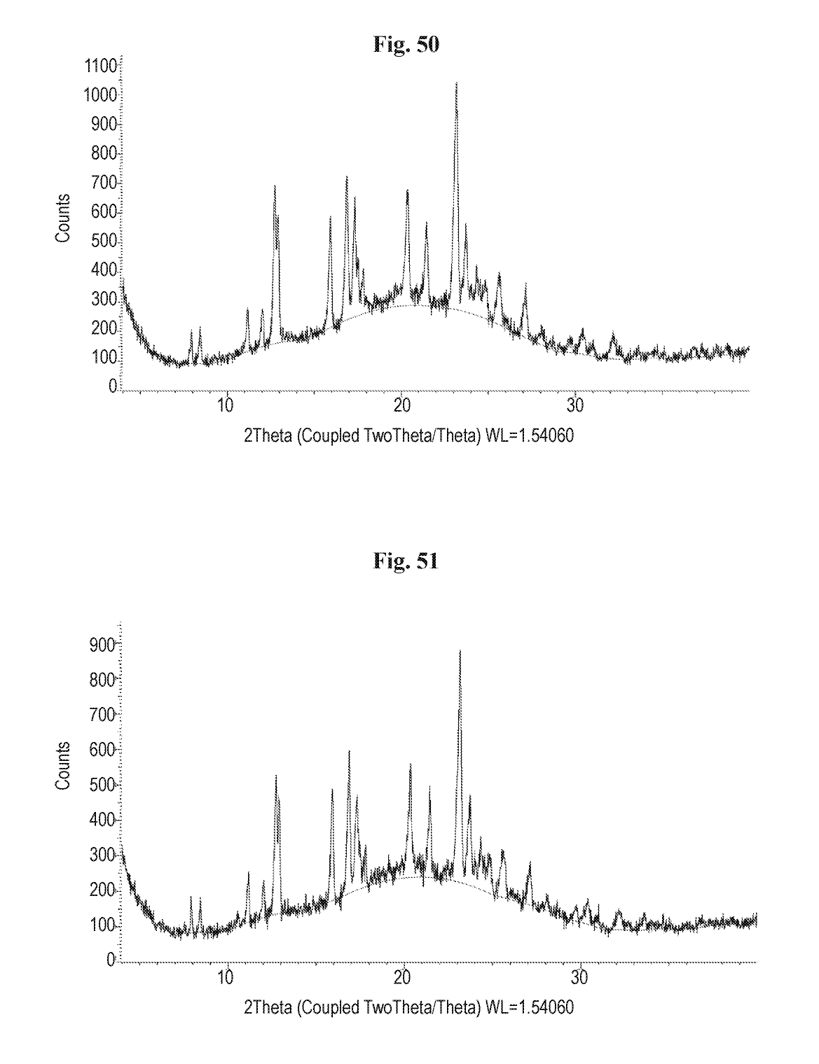

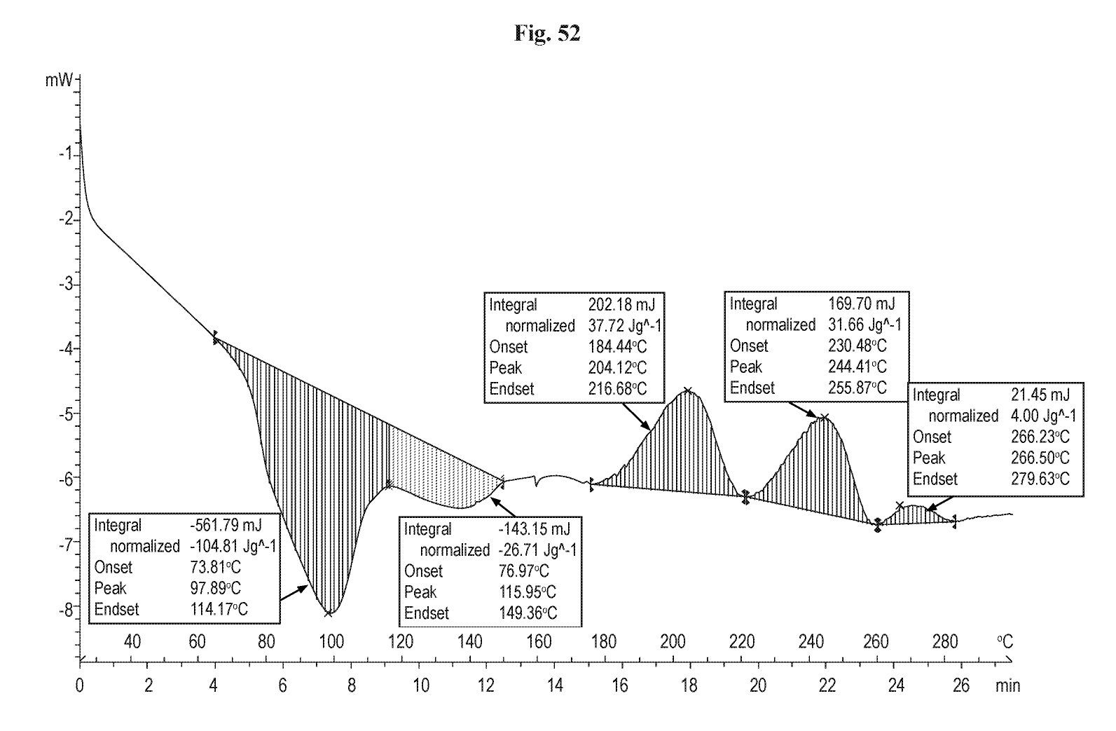

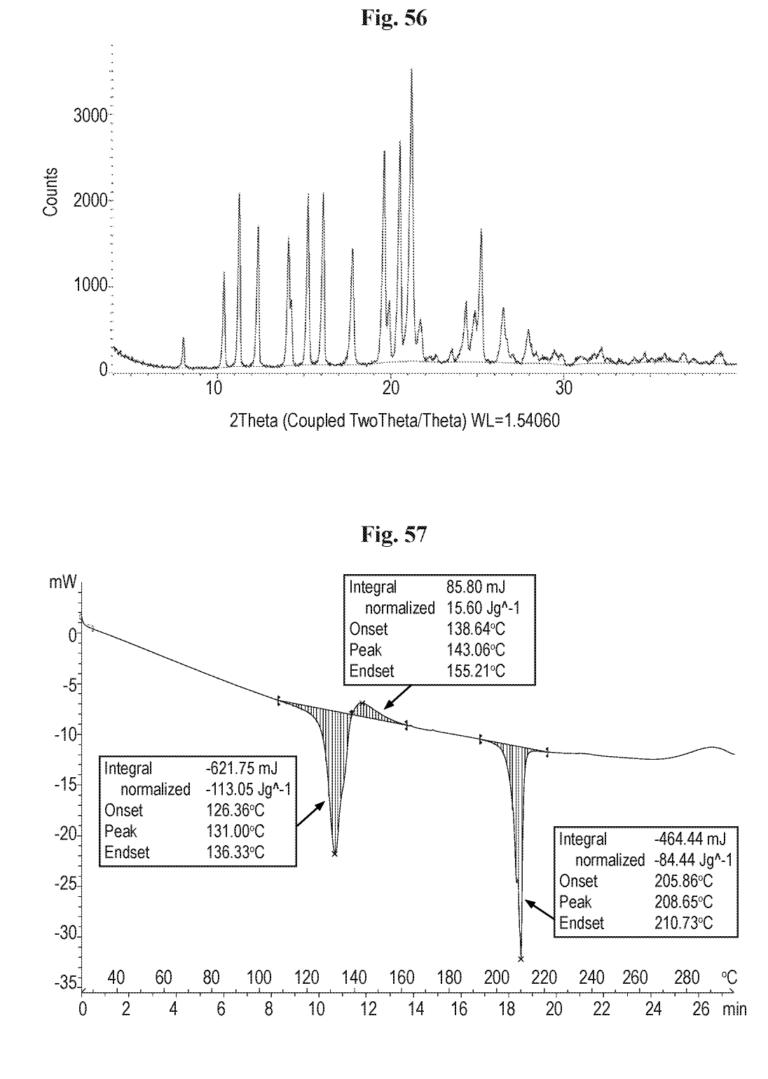

In certain specific embodiments, the present invention provides a solid crystalline form of apomorphine free base or a hydrate or solvate thereof, having an XRPD pattern equivalent to that of FIG. 33, FIG. 34, FIG. 35, FIG. 36, FIG. 37, FIG. 38, FIG. 40, FIG. 41, FIG. 42, FIG. 43, FIG. 44, FIG. 45, FIG. 47, FIG. 48, FIG. 49, FIG. 50, FIG. 51, FIG. 52, FIG. 56 or FIG. 57.

In certain embodiments, the present invention provides a solid crystalline form of apomorphine solvate as defined above, wherein the solvate forming solvent is (C₁-C₈)alkanol, e.g., methanol, ethanol, propanol, IPA or n-butanol, but preferably IPA. In particular such embodiments, the invention provides a solid crystalline form of apomorphine free base•IPA solvate wherein the IPA is about 15% to about 25%, about 16% to about 20%, about 17% to about 19%, or about 18% to about 19%, e.g., about 18.0%, 18.1%, 18.2%, 18.3%, 18.4%, 18.5%, 18.6%, 18.7%, 18.8%, 18.9% or 19.0%, by weight of the crystal. In other particular such embodiments, the invention provides a solid crystalline form of apomorphine free base•IPA mono-solvate, i.e., a solid crystalline form of apomorphine solvate, which comprises about 1 mol of IPA per about 1 mol apomorphine free base.

Such solid crystalline forms, e.g., an apomorphine free base/IPA crystals, may have enhanced stability against discoloration or decomposition relative to amorphous apomorphine free base. In addition, such crystals may desolvate at a temperature of about 110° C.; or melt at a temperature of about 204° C. Solid crystals of apomorphine solvate wherein the solvate forming solvent is (C₁-C₈)alkanol may absorb less than 0.1% w/w water from the air when allowed to equilibrate, as measured by Gravimetric Vapour Sorption (GVS), from 0% to about 90% relative humidity and 25±0.1° C.; or may contain about 0.2% w/w water or less.

In certain specific embodiments, the present invention provides a solid crystalline form of apomorphine solvate wherein the solvate forming solvent is (C₁-C₈)alkanol, having an XRPD pattern with peaks at:
(i) 8.44, 12.73, 15.84, 16.85, 17.24, 20.30, 21.37, 23.16, 23.70, 24.27, 24.82, 25.53, and 27.01 degrees 2-theta;
(ii) 8.48, 11.13, 12.88, 15.96, 16.85, 16.99, 23.69, 25.61, 30.38, and 34.35 degrees 2-theta;
(iii) 7.98, 8.49, 11.17, 12.03, 12.69, 12.88, 15.97, 16.83, 17.00, 17.36, 17.72, 20.31, 21.39, 22.43, 23.02, 23.71, 24.09, 24.85, 25.60, 27.04, 30.35, 32.18, and 34.38 degrees 2-theta;
(iv) 10.585, 11.980, 12.768, 13.091, 14.344, 14.526, 15.596, 15.960, 17.637, 18.446, 18.708, 19.678, 20.224, 20.689, 21.497, 22.467, 24.326, 25.437, 26.387, 27.577, 28.067, 32.313, 28.850, and 24.036 degrees 2-theta; or
(v) 7.962, 10.599, 11.952, 12.778, 14.352, 14.527, 15.608, 15.925, 17.584, 18.375, 18.693, 19.688, 20.249, 20.668, 21.480, 22.159, 22.447, 24.024, 24.365, 25.404, 25.662, 26.428, 27.535, 28.036, 28.896, 29.360, 29.860, 30.262, 31.018, and 32.308 degrees 2-theta;
wherein each peak value is ±0.2 degrees 2-theta.

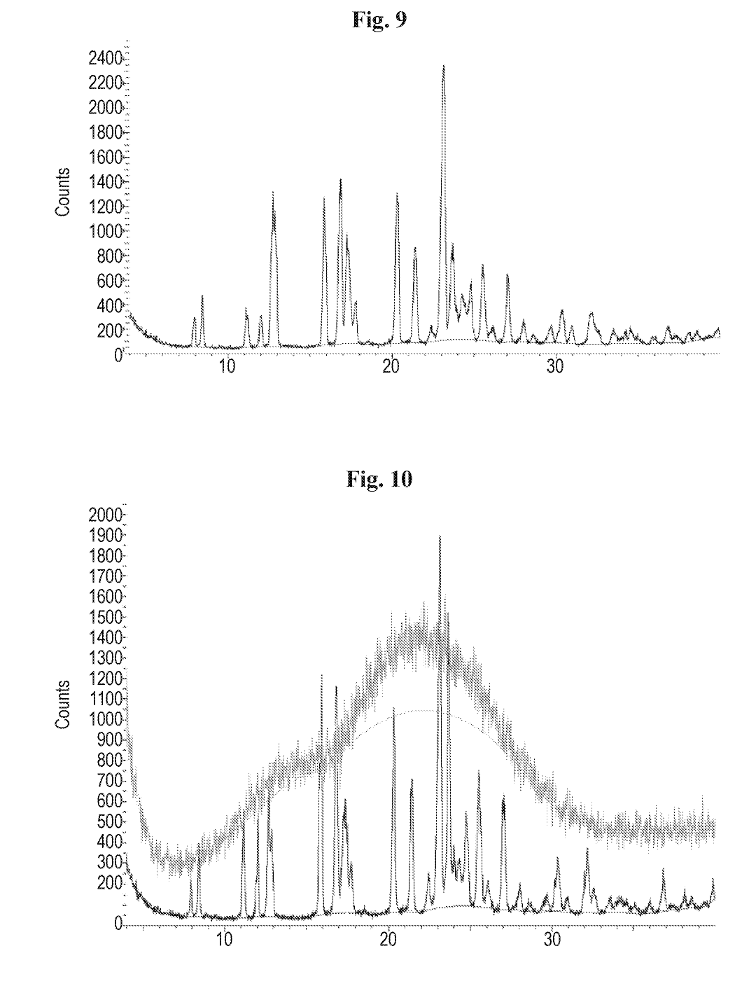

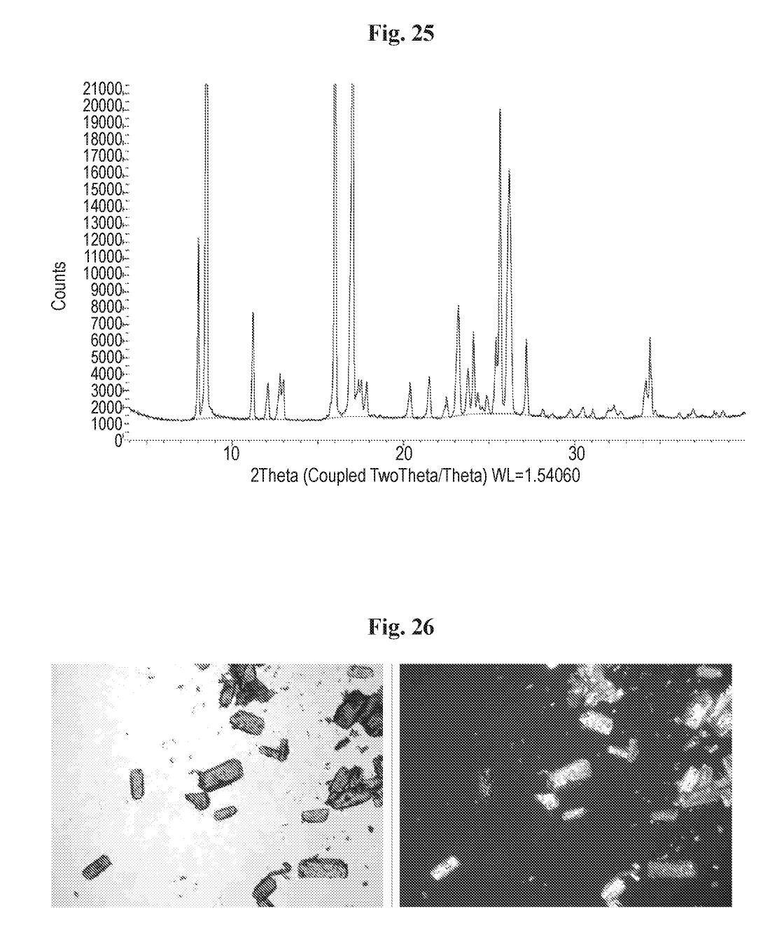

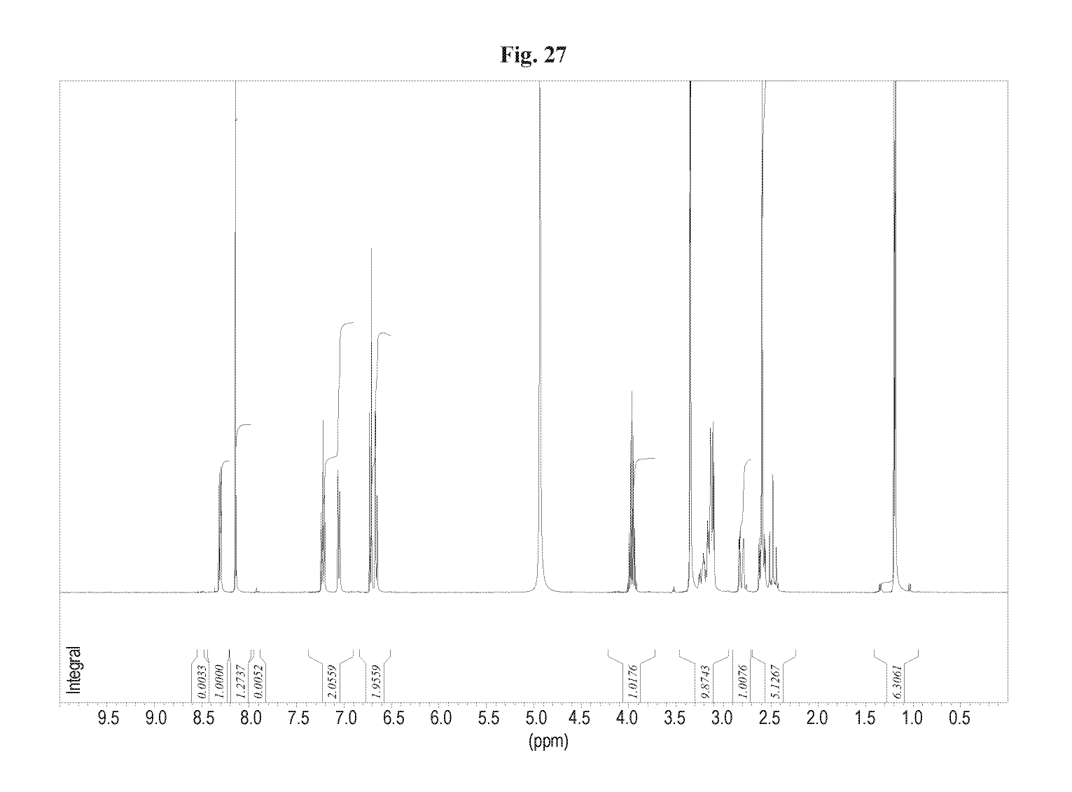

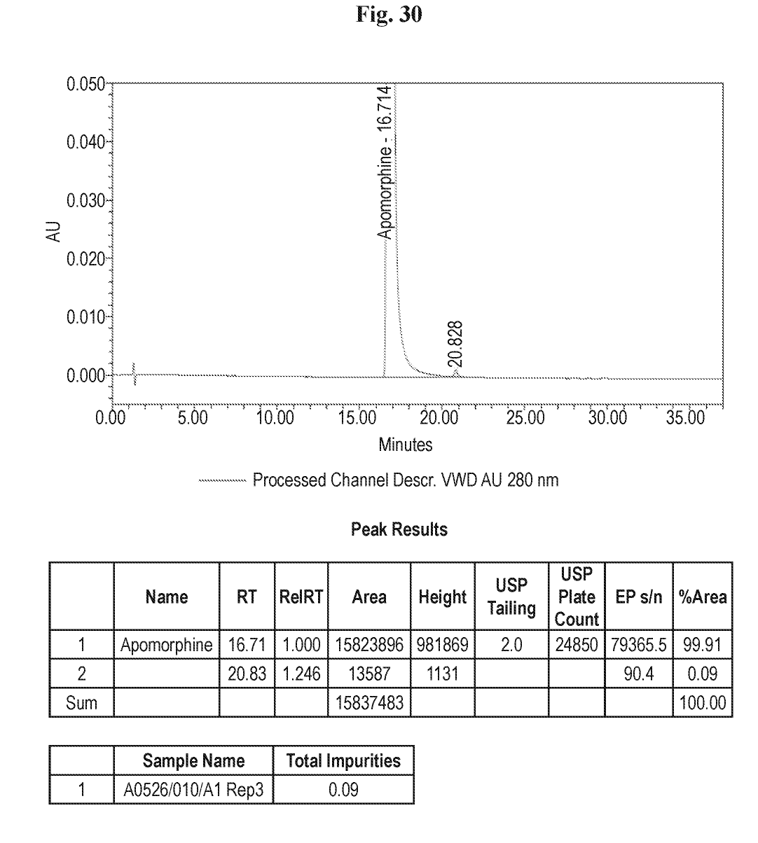

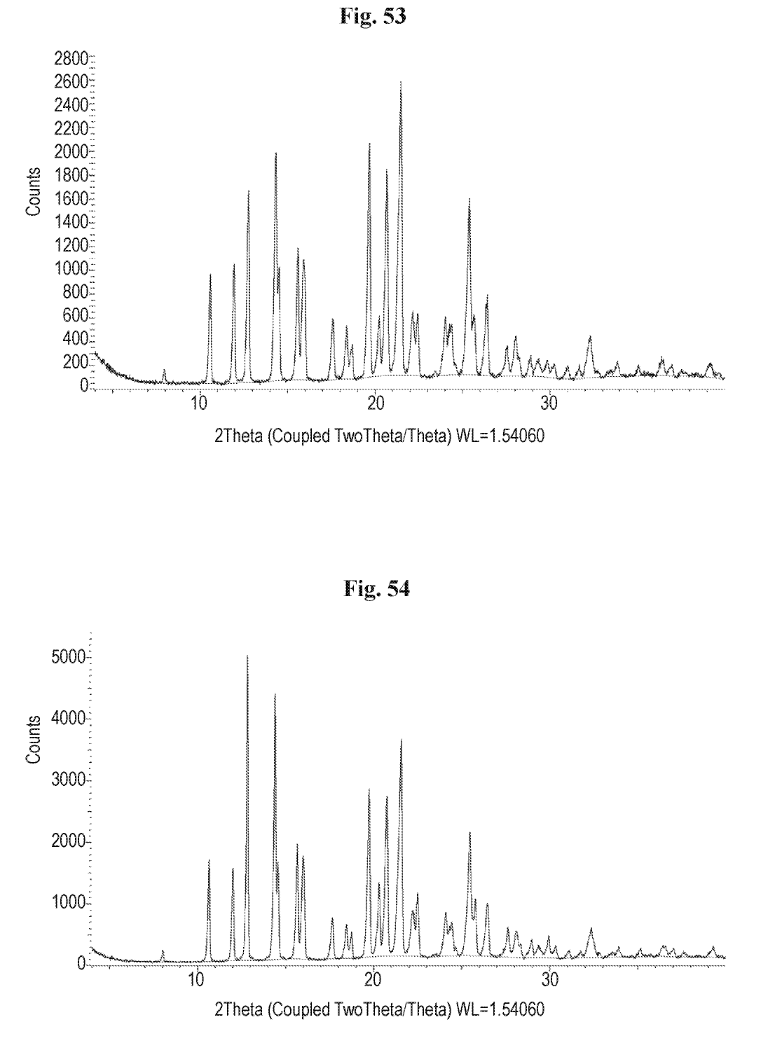

In other specific embodiments, the present invention provides a solid crystalline form of apomorphine solvate wherein the solvate forming solvent is (C₁-C₈)alkanol, having an XRPD pattern equivalent to that of FIG. 9, FIG. 10, FIG. 25, FIG. 29, FIG. 38, FIG. 53, or FIG. 54.

In another aspect, the present invention provides a liquid formulation produced, or obtained, by dissolving a solid crystalline form of apomorphine free base or hydrate, solvate, or co-crystal thereof as disclosed herein, e.g., said solid crystalline form of apomorphine free base or solvate thereof, in a solvent. The liquid formulation of the invention may further comprise an antioxidant, i.e., an agent that inhibits the formation of oxidation products, such as an o-quinone scavenger, a tyrosinase inhibitor, a Cu⁺² chelator and/or a tetrahydroquinoline.

Examples of o-quinone scavengers include, without being limited to, ascorbic acid, an ascorbate such as Na-ascorbate, ascorbic acid-6-palmitate, L-cysteine, N-acetyl cysteine (NAC), glutathione (GSH), or a mixture thereof.

Examples of tyrosinase inhibitors include, without limiting, captopril, methimazole, quercetin, arbutin, aloesin, N-acetylglucoseamine, retinoic acid, α-tocopheryl ferulate, Mg ascorbyl phosphate (MAP), substrate analogues, e.g., sodium benzoate, or L-phenylalanine, or a mixture thereof.

Examples of Cu⁺² chelators include, without being limited to, Na₂-EDTA or Na₂-EDTA-Ca.

Other antioxidants that may be included in a liquid formulation as disclosed herein are dimercaptosuccinic acid (DMSA), diphenylamine (DPA), trientine-HCl, dimercaprol, clioquinol, sodium thiosulfate, triethylenetetramine (TETA), tetraethylene pentamine (TEPA), curcumin, neocuproine, tannin, cuprizone, sulfite salts such as sodium hydrogen sulfite or sodium metabisulfite, di-tert-butyl methyl phenols, tert-butyl-methoxyphenols, polyphenols, tocopherols, ubiquinones, or caffeic acid.

Further antioxidants that may be included in a liquid formulation as disclosed herein are thiols such as aurothioglucose, dihydrolipoic acid, propylthiouracil, thioredoxin, glutathione, cysteine, cystine, cystamine, and thiodipropionic acid; sulphoximines such as buthionine-sulphoximines, homo-cysteine-sulphoximine, buthionine-sulphones, and penta-, hexa- and heptathionine-sulphoximine; metal chelators such as α-hydroxy-fatty acids, palmitic acid, phytic acid, lactoferrin, citric acid, lactic acid, malic acid, humic acid, bile acid, bile extracts, bilirubin, biliverdin, ethylenediaminetetraacetic acid (EDTA), ethylene glycol tetraacetic acid (EGTA), and diethylenetriaminepentaacetic acid (DTPA); sodium metabisulfite, vitamins such as vitamin E, vitamin C, ascorbyl palmitate, Mg ascorbyl phosphate, and ascorbyl acetate; phenols such as butylhydroxytoluene, butylhydroxyanisole, ubiquinol, nordihydroguaiaretic acid, and trihydroxybutyrophenone; benzoates such as coniferyl benzoate; uric acid; mannose; propyl gallate; selenium such as selenium-methionine; stilbenes such as stilbene oxide and trans-stilbene oxide; or combinations thereof.

A liquid formulation contemplated herein may thus comprise one or more antioxidants selected from ascorbic acid, an ascorbate such as Na-ascorbate, L-cysteine, NAC, GSH, Na₂-EDTA, Na₂-EDTA-Ca, or a combination thereof. In particular embodiments, the liquid formulation comprises ascorbic acid, ascorbic acid-6-palmitate, sodium bisulfite, or a combination of ascorbic acid and another antioxidant, e.g., a cysteine such as L-cysteine or NAC. The ratio of ascorbic acid to the other antioxidant, e.g., L-cysteine or NAC, may exist at a particular weight-to-weight ratio such as about 1:1, about 2:1, about 3:1, about 4:1, about 5:1, about 6:1, about 7:1, about 8:1, about 9:1, or about 10:1.

In certain embodiments, the liquid formulation disclosed herein is a pharmaceutically acceptable liquid formulation also referred herein to as a pharmaceutical composition, i.e., a liquid formulation as disclosed in any one of the embodiments described above, when further comprising one or more pharmaceutically acceptable carriers or excipients.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, and the like, that are compatible with pharmaceutical administration. Examples of such excipients include, without limiting, Tween-80, Tween-60, Tween-40, Tween-20, N-methylpyrrolidone (NMP), or polyvinylpyrrolidone (PVP), EDTA or salts thereof, cysteine, N-acetylcysteine, sodium bisulfite, and mixtures thereof. The use of such media and agents for pharmaceutically active substances is well known in the art. The pharmaceutical compositions disclosed herein may contain other active compounds providing supplemental, additional, or enhanced therapeutic functions.

The terms “pharmaceutically acceptable” and “pharmacologically acceptable” as used herein refer to molecular entities and compositions that do not produce an adverse, allergic, or other untoward reaction when administered to an animal or human, as appropriate. For human administration, preparations should meet sterility, pyrogenicity, general safety, and purity standards as required by a government drug regulatory agency, e.g., the United States Food and Drug Administration (FDA) Office of Biologics standards.

The liquid formulations and pharmaceutical compositions disclosed herein may be liquid solutions, i.e., substantially homogeneous liquid mixtures at room temperature (e.g., 25° C.). In particular embodiments, the liquid formulations and pharmaceutical compositions disclosed herein are substantially aqueous.

In certain embodiments, the liquid formulations and pharmaceutical compositions of the present invention are stable for at least 24 hours, 48 hours, or more, i.e., for 1, 2, 3, 4, 5, 6, or 7 days, 1 week, 2 weeks, 1 month, 2 months, or more, at room temperature, e.g., at any temperature in the range of 18° C. to 30° C., e.g., at 25° C.

In certain embodiments, the liquid formulations and pharmaceutical compositions of the present invention have substantially no precipitation of solids for at least 24 hours, 48 hours, or more, i.e., for 1, 2, 3, 4, 5, 6, or 7 days, 1 week, 2 weeks, 1 month, 2 months, or more, at room temperature, e.g., at any temperature in the range of 18° C. to 30° C., e.g., at 25° C.

The pharmaceutical composition of the present invention may be formulated for any suitable route of administration, e.g., for subcutaneous, transdermal, intradermal, intravenous, intraarterial, intramuscular, intraperitoneal, intrathecal, intrapleural, intratracheal, intranasal, sublingual, or buccal administration.

In yet another aspect, the present invention relates to a method of treating a neurological or movement disease or disorder, or a condition associated therewith, in a patient in need thereof, comprising administering to said patient a liquid formulation as disclosed herein. Examples of neurological or movement diseases or disorders include, without being limited to, Parkinson's disease, Alzheimer's disease, and akinesia, and non-limiting examples of conditions associated with neurological or movement diseases or disorders include alcoholism, opiate addiction, and erectile dysfunction. In a particular embodiment, the invention thus relates to a method of treating Parkinson's disease in a patient in need thereof, comprising administering to said patient a liquid formulation as disclosed herein.

In still another aspect, the present invention relates to a liquid formulation as disclosed herein, for use in the treatment of a neurological or movement disease or disorder, or a condition associated therewith. In a particular embodiment, the invention thus relates to a liquid formulation as disclosed herein, for use in the treatment of Parkinson's disease.

In a further aspect, the present invention relates to a method of producing a solid crystalline form of apomorphine free base or a solvate thereof as disclosed herein, said method comprising:
a) dissolving apomorphine hydrochloride and, optionally, an antioxidant such as an ascorbate-based antioxidant, e.g., ascorbic acid-6-palmitate, in a solvent selected from (C₁-C₃)alkyl-, dialkyl-, or trialkylbenzene, pyridine, pyrrole, (C₁-C₃)alkyl-CN, (C₁-C₃)alkyl-NO₂, (R)₂NC(O)H wherein R is H or (C₁-C₆)alkyl, (C₁-C₅)alkylC(O)O— esters such as (C₁-C₅)alkyl-C(O)O—(C₁-C₅)alkyl, (C₁-C₈)alkanol, (C₂-C₈)alkyl-0-(C₁-C₈)alkyl, (C₃-C₈)cyclic ether, (C₃-C₇)cyclic diether, (C₂-C₆)glycol, or a mixture thereof;
b) contacting the solution obtained in (a) with a base in an amount sufficient to generate the apomorphine free base or solvate thereof; and
c) subjecting the solution to conditions that result in crystallization of the apomorphine free base or solvate thereof, thereby producing said crystalline form of apomorphine free base or solvate thereof.

As shown herein and defined by the method of the present invention, the disclosed solid crystalline forms of apomorphine free base or solvate thereof are produced starting from apomorphine hydrochloride, which is dissolved in a solvent. Yet, it should be understood that such solid crystalline forms may further be produced, following a method similar to that disclosed herein, wherein the apomorphine hydrochloride is replaced by apomorphine hydrobromide.

In certain embodiments, the solvent dissolving the apomorphine hydrochloride and optionally said antioxidant in step (a) of the method of the invention is a formamide, acetone, TBME, THF, acetonitrile, nitromethane, pyridine, ethylene glycol, cumene, MeOAc, EtOAc, isopropyl acetate, MeOH, EtOH, IPA, n-propanol, n-BuOH, 1,4-dioxane, or a mixture thereof. In particular embodiments, said solvent is MeOH, EtOH, IPA, n-propanol, n-BuOH, or dioxane, preferably IPA.

In particular embodiments, the method of the present invention thus comprises, in step (a), dissolving apomorphine hydrochloride and, optionally, said antioxidant, in IPA, to thereby obtained, following step (c), a solid crystalline form of apomorphine*IPA solvate.

In certain embodiments, the base contacted in step (b) of the method of the invention with the solution obtained in step (a) has a pKa higher than that of apomorphine, i.e., higher than 8.9. Such a base may be selected from a (C₁-C₈)amino alcohol also referred to as (C₁-C₈)alkanolamine, i.e., an organic compound having an alkane backbone of 1-8 carbon atoms, which contain both hydroxyl and amino (—NH₂, —NHR, and —NR₂) functional groups. Particular such bases include, without being limited to, pyrrolidine, piperidine, 2,2,6,6-tetramethylpiperidine, diethylamine, ethanolamine, 2-(methylamino) ethanol, ethanolamine, 2-amino-1-propanol, 3-amino-1-propanol, alaninol, serinol, 2-amino-1-butanol, 4-amino-1-butanol, arginine, or N-methyl dicyclohexyl amine. In a particular embodiment, said base is 3-amino-1-propanol.

In certain embodiments, step (a) of the method of the invention comprises dissolving apomorphine hydrochloride in a solvent as defined above. In other embodiments, the solid crystalline form produced comprises at least one antioxidant, and step (a) of the method of the invention thus comprises dissolving apomorphine hydrochloride and said antioxidant in a solvent as defined above. The antioxidant may be any antioxidant as defined above, or a mixture of such antioxidants. In particular embodiments, the antioxidant is an ascorbate-based antioxidant such as ascorbic acid-6-palmitate. More particular such embodiments are those where the antioxidant is ascorbic acid-6-palmitate, and the amount of antioxidant dissolved in the solvent is about 0.001% to about 6%, about 0.001% to about 5%, about 0.001% to about 4%, about 0.001% to about 3%, about 0.001% to about 2%, about 0.001% to about 1%, about 0.005% to about 2.5%, about 0.005% to about 2.0%, about 0.005% to about 1.5%, or about 1%, 2%, 3%, 4%, 5% or 6%, by weight, relative to the amount of apomorphine hydrochloride.

In certain embodiments, at least part of the method of the present invention is performed under a flow of an inert gas. In particular such embodiments, the method of the invention is aimed at producing a solid crystalline form of apomorphine*IPA solvate, and step (a) of the method comprises dissolving apomorphine hydrochloride and, optionally, said antioxidant, in IPA, following which the solution is placed under a flow of nitrogen.

In certain embodiments, step (a) of the method of the present invention includes heating the components, i.e., said solvent, apomorphine hydrochloride, and optionally said antioxidant. In particular such embodiments, the method of the invention is aimed at producing a solid crystalline form of apomorphine•IPA solvate, and step (a) includes heating the components to a temperature of about 55° C. to about 83° C.

In certain embodiments, the solution obtained in step (b) of the method of the present invention, which contains the apomorphine free base or solvate thereof, is a homogenous solution. In other embodiments, the solution obtained in step (b) is filtered prior to step (c), i.e., before subjecting to conditions that result in crystallization of the apomorphine free base or solvate thereof.

In certain embodiments, the crystallization step (c) of the method of the present invention comprises gradual cooling over 1 to 24 hours to initiate crystallization. In particular such embodiments, step (c) comprises gradual cooling from approximately 82° C. to 68-72° C. for 1-2 h, and then to approximately 18-23° C. for 3-10 hours.

In certain embodiments, the crystallization step (c) of the method of the invention further comprises seeding the solution with seed crystals to initiate crystallization of the solution.

It should be understood that a variety of crystallization methods and techniques may be used in accordance with the disclosed method. For example, the crystallization of the apomorphine free base or solvate thereof in step (c) may be performed by diffusion techniques; evaporative crystallization, e.g., at a high partial pressure of an inert gas such as nitrogen; slow evaporation under reduced pressure; or a classical crystallization. The crystallization may also be performed by rapid, ballistic, or shock cooling, e.g., to 0° C. or −78 C°.

In yet a further aspect, the present invention relates to a crystalline form of apomorphine free base or solvate thereof produced, or obtained, by a method as disclosed herein.

In still a further aspect, the present invention provides a kit comprising (i) crystals or co-crystals of apomorphine free base or a hydrate or solvate thereof, or a formulation obtained by dissolving said crystals or co-crystals, and optionally an additional therapeutic agent such as levodopa, carbidopa, or entacapone, in a solvent; and optionally (ii) instructions for use. Such a formulation may be liquid as disclosed herein, or in the form of a lyophilized powder that can be reconstituted into a liquid formulation. The formulation may be designed for administration by any suitable route such as, but not limited to, subcutaneously, transdermally, intradermally, intravenously, intraarterially, intramuscularly, intraperitoneally, intrathecally, intrapleurally, intratracheally, intranasally, sublingually, or buccally; or may be designed for transdermal administration and form a part of a transdermal patch.

In certain embodiments, a kit as disclosed herein comprises one, two, three, or more pre-filled cartridges, each containing a liquid formulation as disclosed herein, suitable for use by a patient or a physician. Each such pre-filled cartridge may contain a disclosed liquid formulation comprising a single dose, i.e., a dose suitable for a single administration to a patient, of an apomorphine free base or a solvate thereof, and optionally an additional therapeutic agent, e.g., levodopa, carbidopa, or entacapone.

The invention will now be illustrated by the following non-limiting Examples.

### EXAMPLES

### Example 1: Preparation of Apomorphine Free Base by Aqueous Method

An aqueous procedure was used to isolate apomorphine free base using the following steps. Apomorphine hydrochloride (3.1 g, 1.0 wt) was dissolved in 0.1% w/w aqueous sodium metabisulfite solution (200 ml, 67 vol). 1N aqueous sodium bicarbonate solution was added (33 ml, 11 vol) at approximately 25° C. Stirring continued for approximately 30 minutes at approximately 25° C. The resulting precipitate was filtered using Whatman paper #43 ø 110 mm (1 L Buchner flask) using a vacuum pump. The precipitate was washed with water (2×100 ml, 2×33 vol) and dried under a flow of N₂ using a vacuum pump for 4 h. The expected yield was 83.4%. An improved result was obtained by cooling the precipitate for an additional 20 minutes at approximately 4° C.

### Example 2: Crystallization of Free Base Apomorphine

A crystallization study of the free base was conducted (A0513-36) focusing on removal of color, filterability, and determination of whether the solid was crystalline or amorphous. The apomorphine free base used was an amorphous residue isolated from the resin procedure (A0513-32) described below. This solid was highly colored, underscoring the need for a procedure capable of removing any colored impurities in the solid. Solvents and mixtures of solvents were investigated for crystallization of the free base. The results are summarized in Table 1 below.

TABLE 1
Solvents and mixtures for crystallization of the free base

| Sample | Solvent | Solids if present | Liquors | Solvent content | Comments |
|---|---|---|---|---|---|
| A | THF (10 vol)/ | No solids present | Red solution | Not isolated | Dissolved in THF |
| | TBME (15 vol) | | | | thus TBME added |
| B | 1,4-dioxane | Grey solids | Red solution | 28% dioxane | Initially dissolved, |
| | (10 vol) | (158 mg, 85% | | | crystals formed on |
| | | recovery cor.) | | | 16 h stirring |
| C | Chlorobenzene | Blue gum/oil | Not isolated | Not isolated | Dissolved prior to |
| | (10 vol) | | | | oiling |
| D | EtOH (5 vol) | Minor amount of | Not isolated | Not isolated | Dissolved prior to |
| | | solids | | | minor amount of |
| | | | | | solids forming |
| E | TBME (10 vol) | Slurry (turquoise) | Not isolated | Not isolated | Slurry throughout no |
| | | | | | dissolution |
| F | n-BuOH | Solution (with | Not isolated | Not isolated | Initially dissolved in |
| | (10 vol)/TBME | 5 vol of n-BuOH a | | | 5 vol of n-BuOH then |
| | (20 vol) | gel formed) | | | set as a gum at |
| | | | | | ambient. When |
| | | | | | additional n-BuOH |
| | | | | | added a full solution |
| | | | | | formed. Anti-solvent |
| | | | | | added by no |
| | | | | | precipitate formed |
| G | IPA (10 vol) | White solids | Purple | 18% w/w IPA | Dissolved fully prior |
| | | (163 mg, 88% | solution | | to crystallization |
| | | recovery cor.) | | | |
| H | EtOH (5 vol)/ | Light green solids | Purple | 8% w/w EtOH | Dissolved prior to |
| | TBME (5 vol) | (10 mg, 54% | solution | | minor amount of |
| | | recovery cor.) | | | solids forming, |
| | | | | | TBME added to force |
| | | | | | more product out of |
| | | | | | solution |
| I | THF (5 vol)/ | Light green solids | Purple | — | Dissolved prior to |
| | w-heptane (1 vol) | (104 mg uncor.) | solution | | minor amount of |
| | | | | | solids forming, |
| | | | | | heptane then added |
| J | n-BuOH (10 vol)/ | Minor dark solid | Not isolated | Not isolated | Fully dissolved in n- |
| | n-heptane | | | | BuOH then addition |
| | (21 vol) | | | | of heptane |
| K | Toluene (10 vol) | Green/black oil | Not isolated | Not isolated | Green slurry forms |
| | | | | | on addition on |
| | | | | | heating an oil forms |
| | | | | | no change from |
| | | | | | increasing the solvent |
| | | | | | charge |
| L | MIBK (5 vol) | Green/black oil | Not isolated | Not isolated | Initially forms a |
| | | | | | solution, on stirring |
| | | | | | the product oil, heat |
| | | | | | cool cycles resulted |
| | | | | | in oils |
| M | i-PrOAc (10 vol) | Grey/green solids | Light purple | 1% w/w | Green slurry forms |
| | | 94 mg cor. 51% | | | on addition, heating |
| | | theoretical | | | to reflux not |
| | | | | | dissolved on cooling |
| | | | | | green solids triturated |
| | | | | | to grey/green solids |
| N | DIPE (10 vol) | Green solids | Clear | Not isolated | Solids did not |
| | | | | | dissolved on addition |
| | | | | | of solvent or heating, |
| | | | | | no colour was |
| | | | | | removed to the |
| | | | | | liquors |
| O | MeOAc (7 vol) | Very light green | Dark purple | 20% w/w | On addition of 5 vol a |
| | | solids 172 mg cor. | | | full solution formed |
| | | 93% theoretical | | | and solid then |
| | | | | | precipitated at |
| | | | | | ambient temp. |
| | | | | | Further 2 vol MeOAc |
| | | | | | added to add mobility |
| P | EtOAc (5 vol) | Green solids | Dark purple | 23% w/w | Dissolved slowly on |
| | | 131 mg cor. 71% | | | charge of EtOAc |
| | | theoretical | | | (5 vol), heated to |
| | | | | | obtain full solution. |
| | | | | | Green oil later |
| | | | | | formed on cooling |
| | | | | | which triturated to a |
| | | | | | solid |
| Q | n-BuOAc | Oily black solids | Dark | Not isolated | Dissolved slowly on |
| | (10 vol) | | blue/green | | charge of BuOAc |
| | | | | | (5 vol), heated to |
| | | | | | obtain full solution |
| | | | | | (purple). Green solid |
| | | | | | later formed on |
| | | | | | cooling, no colour in |
| | | | | | liquors |
| R | i-BuOAc | Oily black solids | Dark | Not isolated | 5 vol no dissolution |
| | (10 vol) | | blue/green | | until heated, formed |
| | | | | | a purple solution. |
| | | | | | Gum on cooling, |
| | | | | | attempted higher |
| | | | | | dilution and slow |
| | | | | | cooling, result was |
| | | | | | the same |
| S | MEK (5 vol) | Full solution | Dark | Not isolated | — |
| | | | blue/green | | |
| T | Et₂O (5 vol) | Dark green solids | Light | Not isolated | Initial slurry became |
| | | | green/blue | | immobile, could not |
| | | | | | dissolve on heating in |
| | | | | | 5 vol charged up to |
| | | | | | 25 vol and reflux, |
| | | | | | some dissolution but |
| | | | | | not complete, further |
| | | | | | solids formed on |
| | | | | | cooling |
| U | Anisole (5 vol) | Full solution oiled | Dark | Not isolated | Fill solution |
| | | on extended | blue/green | | remained in solution |
| | | stirring | | | when cooled, later |
| | | | | | oiled in stirring at |
| | | | | | ambient. Heat cool |
| | | | | | cycles and additional |
| | | | | | solvent added, |
| | | | | | remained black/blue |
| | | | | | oil |
| V | Cumene (10 vol) | Dark green solids | Colorless | Not isolated | Melted on heating |
| | | | | | but solid triturated to |
| | | | | | flowing green solid |
| | | | | | on cooling |
| W | Ethyl formate | Green solids | Not isolated | Not isolated | Initially a near full |
| | (10 vol) | | | | solution formed and |
| | | | | | then precipitate |
| | | | | | formed. Heating to |
| | | | | | near reflux formed a |
| | | | | | near solution which |
| | | | | | precipitated on |
| | | | | | cooling to ambient |

Crystallization solvent candidates that resulted in solids include IPA, methyl acetate (MeOAc), ethyl formate, ethanol (at low volumes), ethyl acetate (EtOAc), and isopropyl acetate (i-PrOAc). Crystallization solvent candidates that result in a gum or highly colored solids include: toluene, chlorobenzene, n-butylacetate (n-BuOAc), isobutyl acetate (i-BuOAc), methyl isobutylketone (MIBK), and anisole. Solvents include methanol, n-butanol, THF, MEK. Anti-solvents include TBME, heptane, diisopropyl ether (DIPE), and cumene.

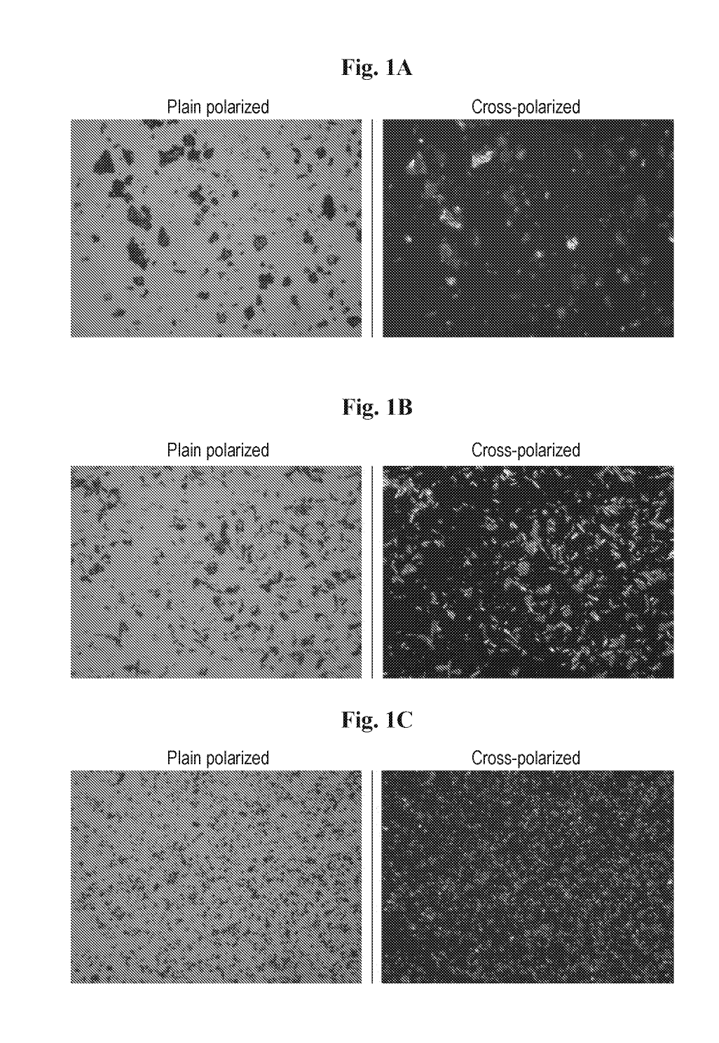

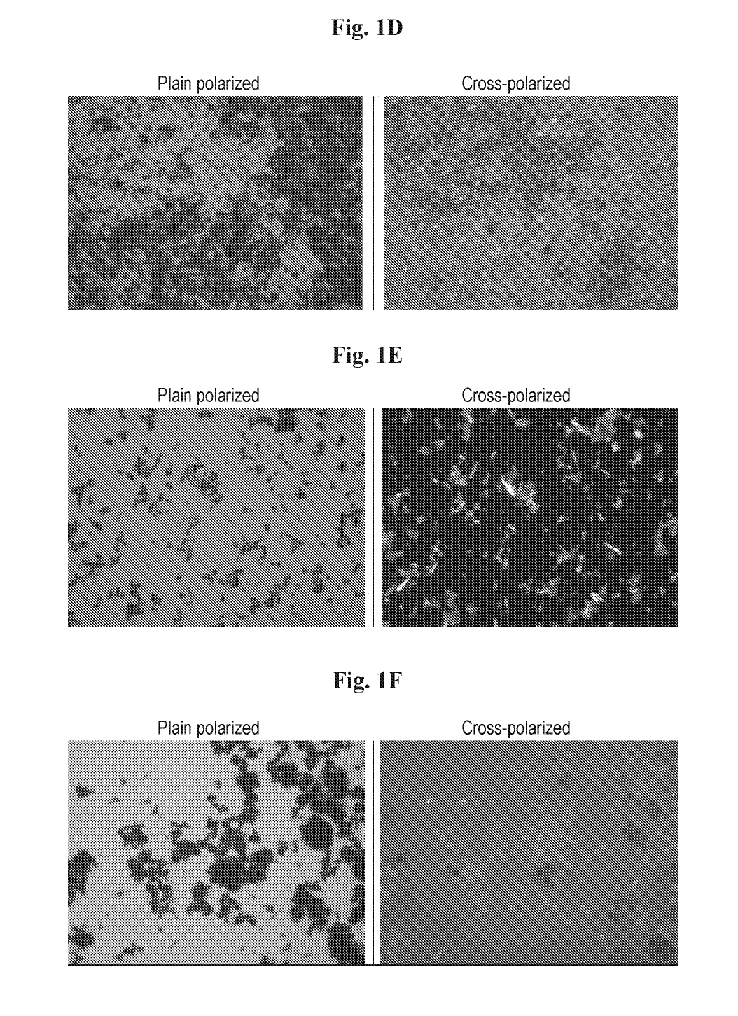

The most promising solids isolated from crystallization solvent candidates were investigated by microscope under plain- and cross-polarized light. These were isolated in good yield and were white to off-white solids (FIGS. 1A-1F).

Four samples (B, G, H and O) were transferred to a vacuum oven and dried at 45° C. for 24 h. Post-drying, solvent contents were 23.4% w/w dioxane, 18.6% w/w IPA, 20.0% w/w MeOAc and 7.8% w/w EtOH, respectively. Crystals isolated from dioxane and IPA were approximately mono-solvates and in the case of EtOH a hemi-solvate. Sample G (from IPA) was heated to 75° C. for 17 h under vacuum, at which point IPA remained at 17.4% w/w, indicating that the solvent is bound in the crystal. MeOAc and IPA resulted in the whitest material, and they had a similar level of crystallinity. These solvents were entrained in the crystal at approximately 1 molar equivalents (eq.), confirming that they were stoichiometric solvates.

Crystals isolated from IPA and 1,4-dioxane were investigated by DSC to determine the temperature at which solvent would be released. IPA is released at >110° C., and dioxane at >120° C. IPA and MeOAc allow full dissolution of the free base at moderate temperature and poor solubility on cooling to ambient temperature. Controlling the rate of cooling can therefore allow control over the rate of crystal growth and thus control of the filtration, which is problematic when following the aqueous route.

Significantly, both IPA and MeOAc allow removal of the green color from the product. IPA was selected as the lead solvent candidate for the final crystallization.

### Stability to Air

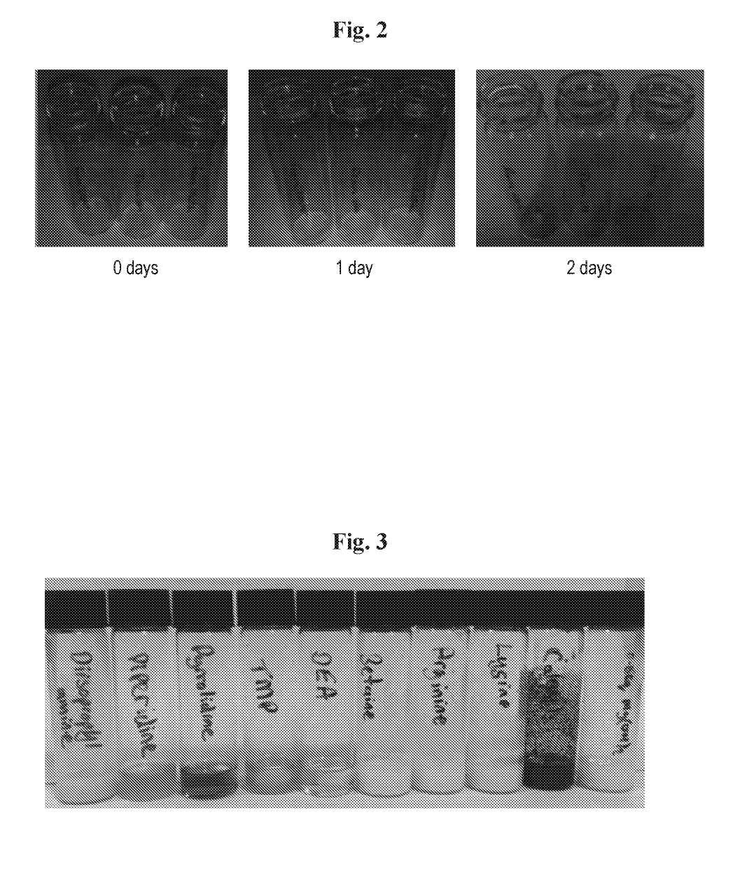

Samples of free base isolated from water, IPA, and MeOAc were retained open to air and the color visually monitored (FIG. 2). Over the course of 2 days, the products isolated from organic solvents discolored to green solids. The material isolated from water began as a yellow/green solid, did not appear to change over these two days, and gradually discolored over the course of about 4 weeks. This extended stability may be due to residual sulfite in the material isolated from water acting as an antioxidant to impart some protection against oxidation to apomorphine free base.

### Example 3: Solubility

A portion of apomorphine free base was synthesized utilizing the supplied aqueous method (A0513-02-01). This material was used in 50 mg portions (A0513-06).

The free base is fully soluble in 5 vol of either MeOH, EtOH, or n-BuOH. Of the solvents screened, n-BuOH appeared promising as a candidate for a reaction solvent, as it did not dissolve the HCl salt (in up to 20 vol at reflux) and could potentially offer both a phase separation with water and an azeotrope with water to dry the organic phase (if required). Methanol was the only solvent that dissolved any significant amount of the HCl salt. The free base precipitated as an oil from most solvents, particularly after heating. An apparently crystalline solid formed following dissolution of the free base and cooling in 1,4-dioxane.

### Example 4: Salt Release Methods

Salt release was accomplished using: 1) basic resins where the resin would be filtered from a solution of the product; 2) organic bases to remove the base hydrochloride salt by precipitation, retaining the apomorphine in solution; and 3) lipophilic bases keeping the base hydrochloride salt in organic solution and obtaining the apomorphine free base by precipitation or extraction.

### Resins

To use resins, solubility of the free base in solution was required, and therefore the alcoholic solvents identified above that are able to dissolve the free base were used. To determine the required loading of resin and the most suitable solvent, a small screen was performed utilizing Amberlite IRA-402(OH) and Ambersep900 hydroxide form resins with MeOH, EtOH, and n-BuOH. To each portion of apomorphine hydrochloride (200 mg, 1.0 wt), Amberlite or Ambersep (100 mg, 0.5 wt) was added in MeOH, EtOH or n-BuOH (7.5 vol).

The noted weights of resins included the weight of water contained in the resin. Prior to use, each resin was washed with solvent (3×10 vol) to remove water. The progress of the reactions could be followed visually as the apomorphine hydrochloride was present as a white slurry (except in MeOH, in which the HCl salt has moderate solubility), while the resin was present as red beads. The white slurry disappeared as the reaction progressed.

Salt release with basic resins was possible and performed well with Amberlite IRA-402(OH) (2.5 wt, wet weight). Ambersep resin required higher loadings to effect the salt release and was therefore not investigated further. Methanol was preferred, as the HCl salt had more solubility in MeOH than the other alcohols, resulting in the fastest reaction rate.

The Amberlite IRA-402(OH) conditions in MeOH were scaled up to 10 g (A0513-32; this material was later used in the initial screening of isolation conditions, see below).

Amberlite IRA-402(OH) (25 g, 2.5 wt) was washed with MeOH (degassed, 4×100 ml, 4×10 vol); 4 washes resulted in 2.0% w/w water. Apomorphine hydrochloride (10.0 g, 1.0 wt) was charged to the resin with MeOH (degassed, 75 ml, 7.5 vol). The mixture was slurried, protected from light and air at 18-23° C. for 30 min, and filtered and washed with MeOH (2×7.5 vol). The blue/violet solution was concentrated to a residue at 45° C. on a rotary evaporator to yield 5.9 g (67%) (A0513-32-01) as a turquoise solid (yield corrected for 7.7% w/w MeOH by ¹H NMR). Liquid chromatography showed 99.68% area vs. 99.73% area when released from via the supplied aqueous route. In an attempt to recover material from the resin, washes with 2 M HCl in MeOH (40 vol) were performed to yield 600 mg of a black tar.

Although a portion of apomorphine free base was generated for use in solubility screening, the recovery from the resin was poor. The yield was low and the material isolated was highly colored, which was challenging to remove by crystallization.

### Removal of Base Hydrochloride by Filtration

A base with a pKa higher than that of apomorphine (pKa 8.9) was used to release salt from apomorphine and to form its own salt (Scheme 1). Some base salts would be insoluble in a solvent which apomorphine free base would be soluble, allowing removal of the base hydrochloride by filtration.

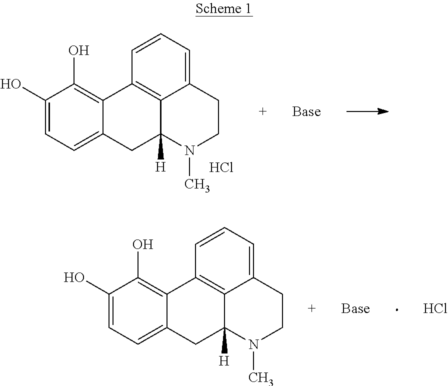 [SMILES: CN1CCc2cccc3c2[C@H]1Cc1ccc(O)c(O)c1-3 ; Cl ; CN1CCc2cccc3c2[C@H]1Cc1ccc(O)c(O)c1-3 ; Cl]

n-BuOH (7.5 vol) was first used as a solvent because it solubilizes the free base but not the apomorphine hydrochloride. The aim was to determine which base hydrochloride salts could be removed by filtration, thus leaving behind a solution of apomorphine free base. A selection of amine bases having pKa values ranging from 10.8 to 13.2 were screened (A0513-10).

The selected base (1.1 eq) was added to a slurry of apomorphine hydrochloride (100 mg) in n-BuOH (7.5 vol). Where a solid formed, it was filtered off and the product-containing liquor analyzed and compared with the expected NMR spectrum of the free base. Where appropriate, the base hydrochloride collected by filtration was also analyzed to determine if the apomorphine hydrochloride was fully released. The results are summarized in Table 2.

The appearance of the salt release reactions is shown in FIG. 3. Salt release was complete with the use of L-arginine, diethyl amine, diisopropylamine, TMP, piperidine, or pyrrolidine. Use of TMP and diisopropylamine resulted in light pink reactions, diethylamine and arginine produced nearly colorless reactions, and lysine yielded a pale yellow reaction (the lysine was pale yellow on addition). Diisopropylamine, lysine, and L-arginine resulted in free base material in the liquors with a solid precipitating.

Of diisopropylamine, L-arginine, and lysine, L-arginine and lysine formed salts that were not soluble in d₆-DMSO. On investigating the solid and the liquor (by NMR), the presence of the L-arginine and lysine could not be detected. Signals were detected for diisopropylamine in the NMR of both the liquors and the isolated solid, indicating that an alternate solvent to n-BuOH may be required in which the diisopropylamine hydrochloride salt is less soluble. L-Arginine was further investigated for the salt release (A0513-50) using MeOH, EtOH, and n-BuOH as reaction solvents (Scheme 2).

MeOH, EtOH and n-BuOH were added to vials (A, B, D and E) with apomorphine as shown in Table 3. HCl (11-00645, Johnson Matthey, 98.9% w/w by NMR assay vs. TCNB) (3×200 mg) and L-arginine (126 mg, 1.1 eq) were added. The reaction was stirred at ambient temperature. Vials D and E were heated to 40° C. to aid solubility of the apomorphine free base. Strong apomorphine signals were detected in the L-arginine removed by filtration by ¹H NMR analysis. The solids were filtered after 2 h of stirring (n-BuOH after 16 h at 40° C.).

The amount of arginine (HCl or free base) retained in the solution of product was found to be least when ethanol was employed as the solvent. Salt release in ethanol at 40° C. was then scaled up to determine the yield more accurately and analyze the viability of the route (A0513-60).

TABLE 2
Observations made during the base screen in n-BuOH

| | | | | | Was |
|---|---|---|---|---|---|
| | | | | Was product | apomorphine |
| | | Observation on | | present in the | free base |
| | Literature | addition of the | Observation on | isolated | present in the |
| Base | pKa | base | aging | solid? | liquor? |
| L-arginine | 13.20 | Remained a white | Remained a white | Weak signals | Concordant |
| | | slurry | slurry | concordant | with free-base |
| | | | | with free-base | |
| Calcium | 12.60 | White slurry green | Green liquor | No solid | Some signals |
| hydroxide | | liquor | deepened to almost | present | present but all |
| | | | black | | aromatic |
| | | | | | signals have a |
| | | | | | shift and poor |
| | | | | | splitting on two |
| | | | | | signals |
| | | | | | assumed due to |
| | | | | | salting of |
| | | | | | phenols |
| Betaine | 12.16 | Remained a slurry | Remained a white | Solids | Consistent with |
| | | | slurry | concordant | incomplete salt |
| | | | | with HCl salt | release |
| Magnesium | 11.40 | Remained a white | Remained a white | Solids | No product |
| hydroxide | | slurry | slurry | concordant | present |
| | | | | with HCl salt | |
| Pyrrolidine | 11.27 | Full dissolution of | Dark green full | No solid | Concordant |
| | | the solids, liquor | solution | present | with free-base |
| | | turned light green | | | |
| Piperidine | 11.22 | Partial dissolution | Light green liquor | No solid | Concordant |
| | | of the solids, | near complete | present | with free-base |
| | | solution light green | dissolution | | |
| TMP | 11.07 | Light slurry | Pink liquor near | No solid | Concordant |
| | | | complete | present | with free-base |
| | | | dissolution | | |
| Diisopropylamine | 11.05 | Partial dissolution | Solids precipitated, | Solids contain | Concordant |
| | | of the solids, liquor | liquor remained | some free- | with free-base |
| | | turned pink | pink | base signals | |
| Diethylamine | 10.93 | Near full solution | Near full solution | No solid | Concordant |
| | | | clear colourless | present | with free-base |
| Lysine | 10.79 | A yellow slurry | Remained a yellow | Solids | Concordant |
| | | | slurry | concordant | with free-base |
| | | | | with HCl salt | |

apomorphine hydrochloride (10.0 g) and L-arginine (6.02 g, 6.0 wt, 1.05 eq) were added to a flask, EtOH (7.5 vol) was added, and the solids were slurried at ambient temperature then heated to 38-42° C. There remained a white slurry throughout. After 1 h at 40° C., ¹H NMR analysis indicated that complete salt release had occurred. Therefore the slurry was filtered at 40° C. and washed with EtOH (2×5 vol). The yield was 4.28 g (48.7%) contained in ethanolic solution A0513-60-02. Assay of the solids indicated 4.53 g (51.5%) contained apomorphine.

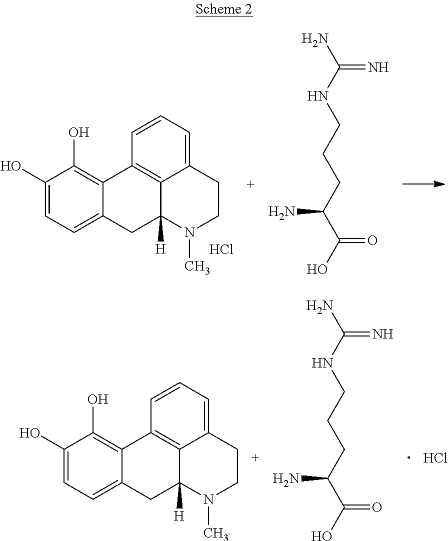

TABLE 3
Yields and arginine contained in the liquors of samples

| Experiment | Solvent | Residue | Solids on filter |
|---|---|---|---|
| A | MeOH | 204 mg uncor contains 10.4 mol %, | 150 mg, contains no |
| | | 6.3% w/w arginine | apomorphine hydrochloride by |
| | | | NMR |
| B | EtOH | 124 mg, 70% theoretical cor. | 237 mg, contains strong |
| | | 1 mol %, 0.6% w/w arginine | apomorphine signals by NMR |
| D | EtOH @ 40° c. | 116 mg, 66% theoretical cor. | 130 mg, contains very |
| | | 1 mol %, 0.6% w/w arginine | weak/minor apomorphine |
| | | | signals by NMR |
| E | BuOH @ 40° c. | 117 mg, 66% theoretical cor. | 137 mg, contains weak |
| | | 59.7% mol %, 25.1% w/w arginine | apomorphine signals by NMR |

The white solids were returned to flask under nitrogen and slurried in EtOH (7.5 vol) at 40° C. After 4 h at 40 to 45° C. the slurry was filtered and a further 1.1 g (12%) was isolated (by solution assay of the liquors). The solids were again returned to the flask and slurried in EtOH (5.0 vol) at 40° C., and after 16 h at 40-45° C. the slurry was filtered and a further 1.1 g (12%) was isolated (by solution assay of the liquors).

The reaction did not fully complete the salt release during the stir period at 40° C., or the product as the HCl salt was encapsulated by the arginine hydrochloride salt. Thus, another approach was employed.

### Removal of Apomorphine Free Base by Filtration

Amines were used to release the apomorphine hydrochloride in IPA, with the objective of leaving the HCl salt of the amine used to release the apomorphine hydrochloride salt in solution with precipitation of the apomorphine free base (A0513-42). The amines chosen below released the apomorphine salt in n-BuOH but remained in solution as their HCl salts (findings from A0513-10). Samples of apomorphine hydrochloride (200 mg) were slurried in IPA (2 ml, 10 vol) and amine base (1.1 eq) added.

NMR analysis indicated that each of the samples had fully released the apomorphine salt. The samples were heated to aid mobility (see Table 4 for observations). All samples were then cooled to 18-23° C. for 2 h and aged for 1 h before filtration. The solids were washed with IPA (2×5 vol) at 18-23° C. and dried for 5 min on the filter.

TABLE 4
Observations on addition of base and heating of samples

| | | | Observation on | Observation on | Observation | Observation |
|---|---|---|---|---|---|---|
| | | | addition at | heating to | on heating to | on heating to |
| | Base | pKa | ambient | about 30° C. | about 40° C. | about 60° C. |
| A | Pyrrolidine | 11.27 | Solids remain white | Remains mobile | Mobile slurry | Thin mobile |
| | | | in green liquor | white slurry in | (green) | slurry (green) |
| | | | | green liquors | | |
| B | Piperidine | 11.22 | Quickly sets to | Remains | Mobile slurry | Thin mobile |
| | | | while gel | immobile white | | slurry (light |
| | | | | gel | | green) |
| C | TMP | 11.07 | Mobile slurry of | Remains slurry | Mobile slurry | Thin mobile |
| | | | white solids in pink | of white solids | | slurry (light |
| | | | solution | in pink solution | | blue) |
| D | Diethyl amine | 10.93 | Light slurry, near | Crystallized | Mobile slurry | Thin mobile |
| | | | solution | form near full | | slurry (light |
| | | | | solution but | | red) |
| | | | | formed an | | |
| | | | | immobile | | |
| | | | | gel/slurry | | |
| E | 3-amino-1- | 10.23 | Forms immobile | Mobile slurry | Mobile slurry | Thin mobile |
| | propanol | | white gel | | (light yellow) | slurry (light |
| | | | | | | green) |
| F | N-methyl- | 9.25 | Forms immobile | Mobile slurry | Mobile slurry | Thin blue |
| | dicyclohexyl | | white gel | | | solution |
| | amine | | | | | |

After storage for 64 h, reactions B and E showed the least green coloration, and C was dark green. Each of the bases effected full salt release of the apomorphine, which precipitated in each case to yield a filterable solid. At ambient temperatures gels formed in some cases but heating resulted in mobile slurries. Therefore, adding the amine to a warm (approximately 40° C.) slurry of the apomorphine hydrochloride was expected to aid mobility and the crystallization of the product. Only reaction C with TMP resulted in significant entrainment of the base used to release the apomorphine in the product (Table 5).

TABLE 5
Yield summary and base removal by NMR analysis

| | | | | Amount of |
|---|---|---|---|---|
| | | | Solvent | base detected |
| | | | content | in solid |
| | | Yield | by ¹H NMR | by ¹H NMR |
| | Base | (% recovery) | (% w/w) | (% w/w) |
| A | Pyrrolidine | 133 mg (76) cor. | 19.0 | 0.04 |
| B | Piperidine | 134 mg (76) cor. | 17.2 | 0.03 |
| C | TMP | 128 mg (73) cor. | 17.8 | 17.57 |
| D | Diethyl amine | 130 mg (74) cor. | 18.7 | 0.03 |
| E | 3-amino-1-propanol | 128 mg (73) cor. | 17.9 | 0.04 |
| F | N-methyl- | 122 mg (70) cor. | 17.9 | <0.00 |
| | dicyclohexyl amine | | | |

From the selection of bases reported above, piperidine and 3-amino-1-propanol resulted in white apomorphine with no base detected in the isolated product by ¹H NMR analysis. Because 3-amino-1-propanol is of lower toxicity, it was chosen for the scale-up reaction. Scheme 3 shows the initial salt release scale-up reaction conditions.

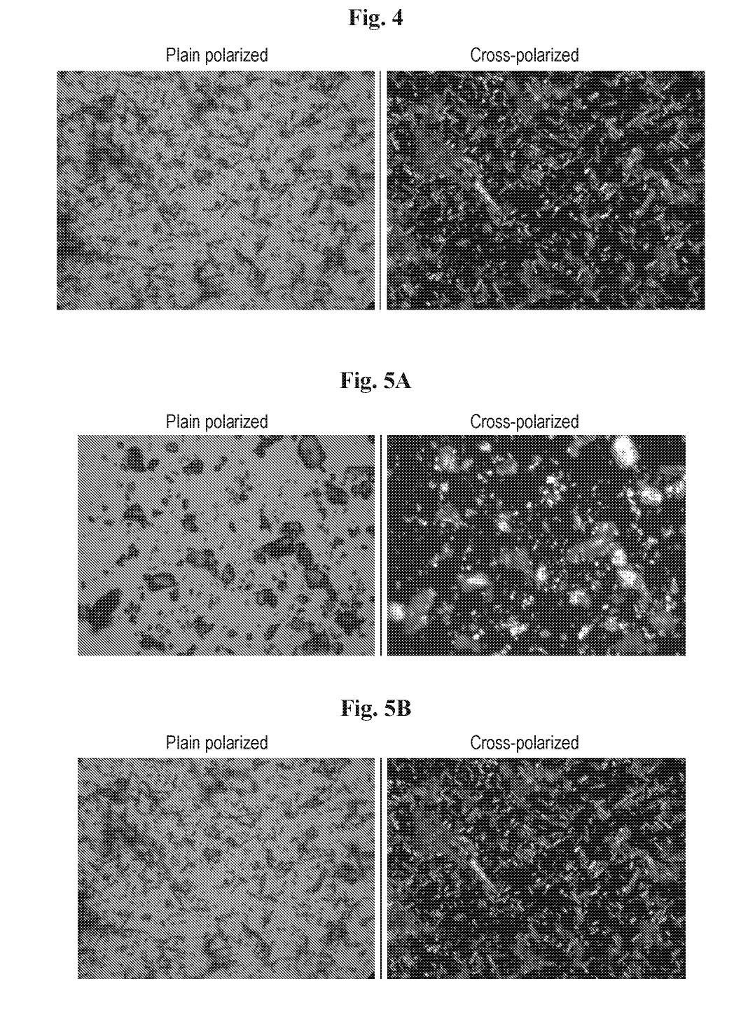

The 3-amino-1-propanol mediated salt release of apomorphine was scaled to 10 g in IPA (10 vol), and in order to aid the mobility of the reaction, the addition of the 3-amino-1-propanol was performed at 40-45° C. On rapid addition a full solution formed from which precipitated the product after approximately 15 min. Analysis of the liquor indicated that 80% crystallization had occurred (later reactions indicated that when the addition time of the 3-amino-1-propanol was increased, the HCl salt converted to the free base without formation of a solution). The slurry was cooled and aged over 16 h at 18 to 23° C. The solid was filtered and washed with IPA to yield a white solid in 92.0% yield corrected for 84.7% w/w assay (the combined mother liquors and washes contained 6.2% yield by assay) (17.8% w/w IPA, approximately 1 eq) 100% area by HPLC KF=0.1% w/w. FIG. 4 shows photo micrographs of the isolated material at ×100 magnification under plain- and cross-polarized light. This material (A0513-64-05) was left in an open vial in a fume hood and its color change and HPLC purity profile monitored over time.

The scale-up reaction indicated that apomorphine free base mono-IPA solvate product could be isolated as a white crystalline solid. The solid was found to contain no residual 3-amino-1-propanol by ¹H NMR.

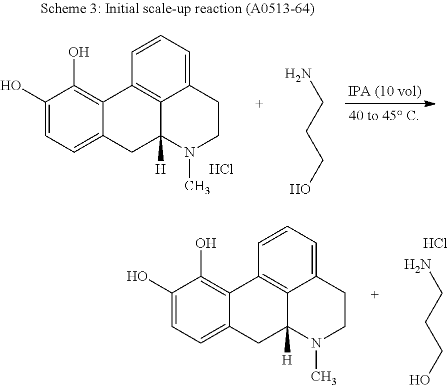

### Recrystallization of Apomorphine Free Base

The stability of the product to air oxidation is expected to increase by increasing the particle size. This reduces the total surface area of the product relative to mass and results in less crystal/air contact. A recrystallization would also be useful as a purification method if ascorbic acid-6-palmitate or 3-amino-1-propanol were entrained in the isolated product. The recrystallization of isolated apomorphine free base was performed to determine the volume of IPA required and the potential seed point if required (A0513-90-01).

On a 1.0 g scale (The material used was A0513-68-01, previously isolated from IPA in the presence of ascorbic acid-6-palmitate), the apomorphine free base was slurried in IPA (10 ml, 10 vol). The slurry was vacuum/nitrogen purged three times at ambient temperature and heated to 50° C. At this temperature, a slurry remained, and only on achieving reflux and adding further IPA (1 vol) was full dissolution obtained. On cooling, solids formed at 71° C.; based on this a seed temperature of 77-78° C. was calculated (later experiments indicated that the optimal temperature for seeding the reaction mixture was 71° C., the difference is likely to be due to the presence of the 3-amino-1-propanol hydrochloride salt). This should allow seeds to be added and not dissolve which will initiate crystal growth and potentially generate larger crystals. The slurry was warmed to 74° C. and held for 1 h before cooling to 55° C. The slurry was held for 40 min before cooling to 18-23° C. for 2 h. The slurry was filtered and resulted in a yield of 1.00 g, 83.5%, 83.5% w/w assay, 16.8% w/w IPA (some mechanical losses were made in transfer to the filter). Chemical purity (CP)=100% area.

The product was stable under the crystallization conditions over the 4 h time period, and no color was observed in the liquor. The isolated material was investigated by microscopy (FIGS. 5A-5B). The recrystallized material had a crystal size larger than that of the material isolated following a 45° C. reaction. The product was a stoichiometric solvate as previously observed.

### Example 5: Antioxidant Additives

The apomorphine free base isolated as an IPA solvate was found to be relatively stable in air when compared to the aqueous route. However, the free base in solution is susceptible to aerial oxidation. To improve stability during processing, ascorbic acid-6-palmitate or BHT were assessed re: stabilizing the apomorphine free base in solution (A0513-54).

200 mg portions of apomorphine free base (isolated by the supplied aqueous route, A0513-02-01) and antioxidant (10 wt %, 20 mg) were added to vials, followed by MeOH (10 vol). The vial was flushed with air, sealed, and stirred. The reactions were compared with a control reaction having no antioxidant.
A—in MeOH (10 vol) (control reaction)
B—ascorbic acid-6-palmitate (10 wt %) in MeOH (10 vol)
C—BHT (10 wt %) in MeOH (10 vol)

Samples were analyzed by HPLC. FIG. 6 shows the color changes over time of reactions A-C. It was found that upon addition, the ascorbic acid-6-palmitate turned the solution from green to yellow. The HPLC profile just after addition of the antioxidant was of similar purity for each sample. After 24 h, sample B had precipitated a small amount of solid in a pale green solution. The HPLC profile of this slurry indicated that the purity was higher than for either A or C. Key impurities elute early in the HPLC profile at RRT 0.15 and 0.18. In the samples without ascorbic acid-6-palmitate these impurities were >1.5% area.

After 72 h, all samples had dark-colored precipitate present. Sample B exhibited the highest purity, while samples A and C continued to degrade to early running impurities totaling >3.1% area and >4.3% area. Further impurities formed at RRT 1.09, 1.25 and to a lesser extent at RRT 1.37, 1.71, and 1.76.

The ascorbic acid-6-palmitate had a stabilizing effect on the apomorphine free base, and prevented discoloration for a limited time, likely due to full consumption of the ascorbic acid-6-palmitate.

### Ascorbic Acid-6-Palmitate as an Antioxidant

The effect of adding ascorbic acid-6-palmitate on stability of the reaction mixture and the isolated solid was determined in a larger scale reaction (A0513-68). On a 10 g scale, ascorbic acid-6-palmitate (0.01 wt) was added to a flask with the apomorphine hydrochloride salt and IPA (10 vol). 3-amino-1-propanol (2.5 ml, 1.1 eq, 0.25 vol) was added at 40-45° C., and a slurry was present throughout. The slurry was stirred at 40-45° C. for 2 h; at this point 10% of the reaction mixture was removed for an extended stability study at 40-45° C. The remainder of the reaction was cooled and aged over 16 h to 18-23° C. The solid was filtered and washed with IPA (2×3 vol) and resulted a white solid (liquor was pale yellow). The solid was dried under a flow of nitrogen for 30 min. The yield was 7.03 g (79.8%, corrected for 18.3% w/w IPA, approximately 1 eq), 100% area by HPLC (Note: 10% of the reaction mixture was removed for the stability study, therefore this yield is at the expected approximately 90% level, and the combined mother liquor and washes contained 7.6% of theoretical yield by assay). In this material the ascorbic acid-6-palmitate and 3-amino-1-propanol in the product were below detectable levels by ¹H NMR analysis.

### Stability of the Reaction Mixture

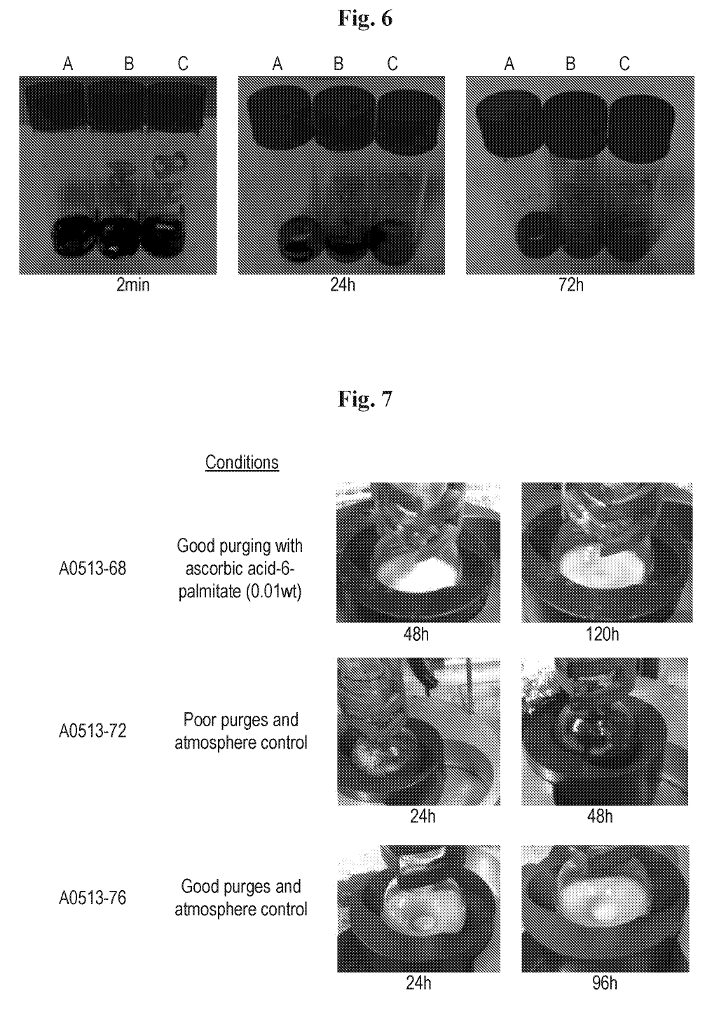

Stability of the reaction mixture was compared with and without ascorbic acid-6-palmitate. Each reaction was held at 40-45° C. and both the color and the HPLC profile monitored. HPLC purity profiles are shown on Table 6. Photographs of the reactions after extended time periods are shown in FIG. 7.

Effective purging of the reaction to remove air is important to obtain a stable reaction mixture at 40-45° C. Even with good purging, minor discoloration is observed over extended stir periods at 40-45° C. Ascorbic acid-6-palmitate resulted in less discoloration but little difference in HPLC profile over the time period examined.

### Stability of the Reaction Mixture at Reflux

Following the observation that a controlled cool down from reflux resulted in a larger particle size, an investigation of the stability under reflux conditions in IPA (10 vol) was performed. In this case, the base was added under reflux and the procedure compared with and without ascorbic acid-6-palmitate as an antioxidant (Table 7).

TABLE 6
HPLC profiles of stability experiments

| | | % area: RRT (RT = 16.43 min, apomorphine method) | | | | | | | | |
|---|---|---|---|---|---|---|---|---|---|---|
| | Comment | 0.74 | 1.00 | 1.09 | 1.22 | 1.25 | 1.37 | 1.50 | 1.71 | 1.76 |
| A0513-68A-03 | Stability of reaction mixture | nd | 99.93 | nd | nd | nd | nd | nd | 0.05 | 0.03 |
| | (40° C., ascorbic | | | | | | | | | |
| | acid-6-palmitate, 24 h) | | | | | | | | | |
| A0513-68A-04 | Stability of reaction mixture | nd | 99.96 | nd | nd | nd | nd | nd | 0.04 | nd |
| | (40° C., ascorbic | | | | | | | | | |
| | acid-6-palmitate, 48 h) | | | | | | | | | |
| A0513-68A-05 | Stability of reaction mixture | nd | 99.86 | nd | nd | 0.04 | nd | nd | 0.06 | 0.04 |
| | (40° C., ascorbic | | | | | | | | | |
| | acid-6-palmitate, 120 h) | | | | | | | | | |
| A0513-72-01 | Stability of reaction mixture | 0.22 | 98.18 | 0.13 | 0.44 | 0.10 | 0.15 | 0.07 | nd | nd |
| | (40° C., poor purging, 24 h) | | | | | | | | | |
| A0513-72-02 | Stability of reaction mixture | 0.10 | 94.66 | 2.32 | 0.42 | 1.46 | 0.16 | 0.10 | nd | nd |
| | 40° C., poor purging, 48 h) | | | | | | | | | |
| A0513-76-01 | Stability of reaction mixture | nd | 99.90 | nd | nd | nd | nd | nd | 0.06 | 0.04 |
| | (40° C., effective purging, 24 h) | | | | | | | | | |
| A0513-76-02 | Stability of reaction mixture | nd | 99.90 | nd | nd | nd | nd | nd | 0.06 | 0.04 |
| | (40° C., effective purging, 96 h) | | | | | | | | | |

nd—not detected.

TABLE 7
Comparative data for reaction in IPA with/without ascorbic acid-6-palmitate

| | | % area: | | | | | | |
|---|---|---|---|---|---|---|---|---|
| | | RRT (RT = 16.43 min, apomorphine method) | | | | | | |
| | Comment | 1.00 | 1.09 | 1.25 | 1.29 | 1.50 | 1.71 | 1.76 |
| A0513-94-01 | Solution at reflux | 99.56 | 0.05 | 0.06 | nd | nd | 0.06 | 0.04 |
| A0513-94-02 | Liquors | 92.99 | 1.79 | 0.57 | 1.20 | 0.22 | 0.34 | 0.43 |
| A0513-94-03 | Isolated solid | 99.81 | nd | 0.13 | nd | nd | 0.02 | nd |
| A0513-98-01 | Solution at reflux with | 99.83 | nd | 0.04 | nd | nd | 0.06 | 0.04 |
| | ascorbic acid-6-palmitate | | | | | | | |
| A0513-98-02 | Liquors | 95.62 | 1.25 | 0.19 | 0.61 | 0.12 | 0.45 | 0.34 |
| A0513-98-03 | Isolated solid | 99.93 | nd | 0.07 | nd | nd | nd | nd |

nd—not detected.

The reaction without ascorbic acid-6-palmitate was found to be unstable and resulted in a green solution after 2 h. This instability was unexpected as the recrystallization experiment discussed above, which employed high-purity apomorphine free base previously isolated from IPA, did not indicate that the product was particularly unstable. It was therefore concluded that the presence of the 3-amino-1-propanol led to the reduced stability under reaction conditions. Addition of ascorbic acid-6-palmitate to the reaction mixture from the start resulted in a clear pale yellow solution after 2 h at reflux, and the solid later isolated was whiter than that isolated in the absence of the ascorbic acid-6-palmitate. The use of 0.01 wt % of the antioxidant was therefore added to the process. This addition was shown to have no detrimental effect on the reaction and to be removed beyond the limit of ¹H NMR detection levels on isolation from IPA. Although the color difference was significant, the difference in the HPLC profile was minor; the isolated yields were also comparable at approximately 85% each.

### Stability Study of the Isolated Solid

Samples of solids isolated from an IPA recrystallization of the free base, salt released via the 3-amino-1-propanol route were monitored by their color change and their HPLC profile. The HPLC profiles are described in Table 8. Photographs of the color changes are shown in FIG. 9.
A0513-64-05: contains no ascorbic acid-6-palmitate.
A0513-68-01: contained ascorbic acid-6-palmitate at 0.1 wt % loading in the reaction, but was not detected by ¹H NMR analysis in the solid output.
A0513-106-02: isolated from IPA (30 vol, reaction contained ascorbic acid-6-palmitate at 0.1 wt % loading).

TABLE 8
HPLC profiles of stability test to air and light

| | | % area: | | | | | | |
|---|---|---|---|---|---|---|---|---|
| | | RRT (RT = 16.43 min, apomorphine method) | | | | | | |
| | Comment | 0.41 | 1.00 | 1.09 | 1.71 | 1.76 | 2.02 | 2.04 |
| A0513-64-05 | Output | nd | 100.00 | nd | nd | nd | nd | nd |
| A0513-64-05a | Isolated stability to air | nd | 99.98 | nd | 0.02 | nd | nd | nd |
| | and light (2 days) | | | | | | | |
| A0513-64-05b | Isolated stability to air | nd | 99.95 | nd | 0.03 | 0.02 | nd | nd |
| | and light (5 days) | | | | | | | |
| A0513-64-05c | Isolated stability to air | 0.03 | 99.92 | nd | 0.02 | 0.03 | nd | nd |
| | and light (17 days) | | | | | | | |
| A0513-68-01 | Isolated from reaction | nd | 99.98 | nd | 0.02 | nd | nd | nd |
| | containing asc (1 day) | | | | | | | |
| A0513-68-01a | Stability of solid in air | nd | 99.97 | nd | 0.03 | nd | nd | nd |
| | and light (5 days) | | | | | | | |
| A0513-68-01a | Stability of solid in air | 0.01 | 99.92 | nd | 0.03 | 0.02 | 0.02 | nd |
| | and light (14 days) | | | | | | | |

nd—not detected.

The isolated product IPA solvate was found to be stable at ambient conditions when exposed to air and light when isolated from reactions with 3-amino-1-propanol used as the base. This is in contrast to materials isolated in this manner using alternative bases (i.e., pyrrolidine, diethylamine, N-methyl dicyclohexylamine, or TMP). This suggests that the base used, and efficient removal of it, and/or the highly crystalline form of the solvate, can be important to the stability of the IPA solvate.

### Example 6: Non-Aqueous Procedure

An optimized non-aqueous procedure is provided below and shown in Scheme 4.
1. Add apomorphine hydrochloride (1.0 wt), ascorbic acid-6-palmitate (0.01 wt), IPA (30 vol) and stir at 18-23° C.
2. Vacuum/nitrogen purge the vessel 3× at 18-23° C. and place under a flow of nitrogen.
3. Heat the slurry to 60-65° C.
4. Add 3-amino-1-propanol (1.1 eq, 0.25 vol) maintaining 60-70° C. (target 60-65° C.).
5. As required, cool the reaction mixture to 60-65° C. (Note: the reaction mixture has been held at this point for 2 h at reflux (83° C.) with no significant change in purity profile (A0513-98))
6. Stir for 15-20 minutes and check for full dissolution.
7. On full dissolution, clarify the reaction mixture through a ≦1 μm filter and line rinse with IPA (3 vol) at 60-65° C. (Note: the reaction solution is highly unstable to air while hot, therefore total exclusion of air is required.)
8. Concentrate the reaction mixture under reduced pressure to approximately 10 vol maintaining 55-65° C.
9. Heat the slurry to reflux (expect 82° C.) and check for full dissolution (Note: the distillation has been performed over 6 h at 60-65° C. with no significant change in purity profile of the material isolated. Minor discoloration of the slurry was observed; pale yellow (A0513-126))
10. Cool the slurry to 68-71° C. and age for 1 h (crystallization is expected during the cool down period approximately 72° C.).
11. Slow the stirring for the remainder of the preparation to the minimum effective rate and check for crystallization: if crystallization has not occurred, seed the reaction with 0.1 wt % of seeds and age for 1 h.
12. Cool the slurry to 18-23° C. over 5-6 h at an approximately constant rate.
13. Age the slurry at 18-23° C. for 2-16 h.
14. Filter the solid and wash with IPA (2×3 vol) at 18-23° C. under nitrogen.
15. Dry the solid under a flow of nitrogen for at least 30 min.
Expected yield: 88-94%.

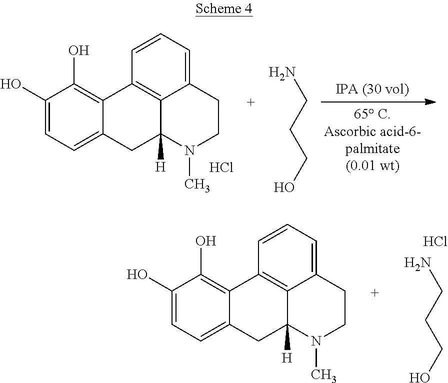

### Scale-Up of Non-Aqueous Procedure

The procedure above was used to prepare a 30 g sample for use in accelerated stability testing. The preparation was performed as described above (A0513-126).

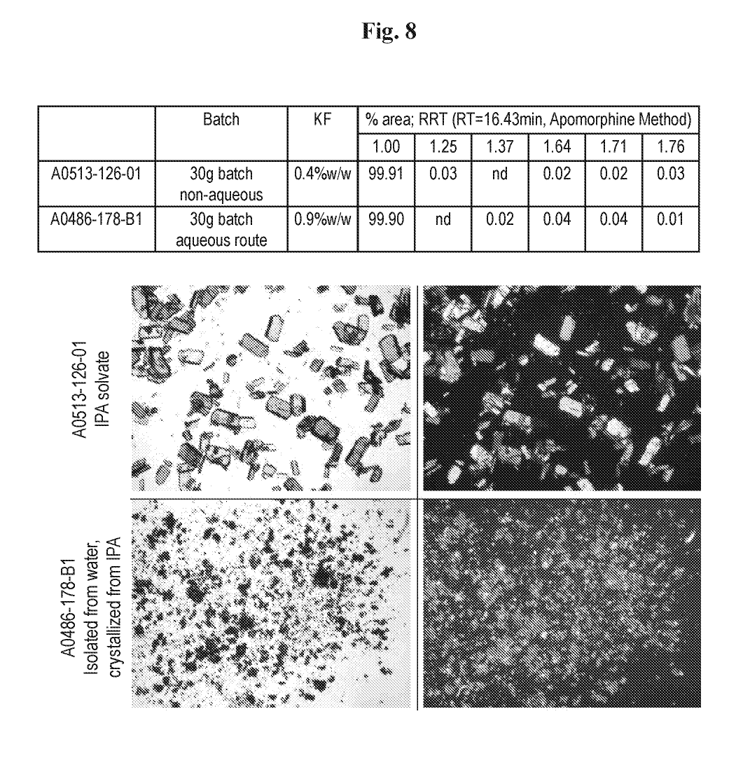

Key points of note: (i) No exotherm was noted on addition of the 3-amino-1-propanol. (ii) During the crystallization phase of the preparation the slurry was heated to 82° C. and full dissolution was obtained. During the cooling step to 71° C. for 1 h the precipitate was noted at 72° C. (iii) The slurry was cooled to 18-23° C. at the rate of approximately 10° C./h. After 5 h the temperature was 21° C. and the slurry was aged for 2 h. (iv) Output: 34.11 g, 86.1% corrected for 17.9% w/w IPA (A0513-126-01). (v) The process performed as expected except for a marginally suppressed yield, which may be due to a shorter age period at ambient temperature. The material was isolated as a highly crystalline solid (approximately 100×250 μm in size; see FIG. 8 and FIG. 10).

### Example 7: Scale-Up of Aqueous Route

The aqueous route described above was performed to prepare a 30 g sample. Scheme 5 shows the reaction conditions for the aqueous route. The preparation was performed as described above (A0513-132; XRPD data shown in FIG. 10).

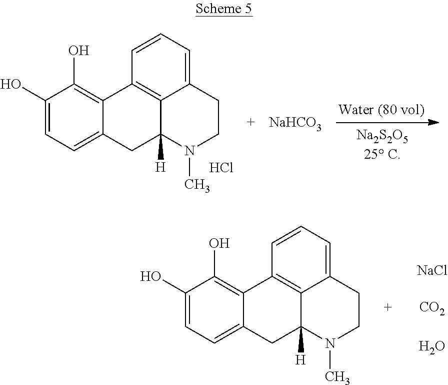

Key points of note: (i) The precipitate was washed with degassed water (2×1.5 L, 2×33 vol) and dried under N₂. Drying of the filter cake was slow considering the scale: 4 h KF=66% w/w water; 6 h KF=23% w/w water; 8 h KF=22% w/w water; 10 h KF=4% w/w water; 12 h KF=2.3% w/w water. (ii) Drying was stopped. Following removal of the solid from the filter, the solid was homogenized and KF was found to be 5.7% w/w. The solid was returned to the filter for further drying: 1 h KF=4.0% w/w; 2 h KF=3.8% w/w; 2.5 h KF=4.3% w/w drying was stopped as a minor amount of pale green was noted in the filter cake; Output: 29.7 g, 75% uncorrected for 4.3% w/w water (A0513-136-07).

The isolated material was crystallized from IPA.

A0486-178—Recrystallization of Crude A0513-136-07 from IPA
1. The crude product (29.7 g, A0513-136-07) was charged to a flask and ascorbic acid-6-palmitate (297 mg, 0.01 wt, 1.0% w/w) was added with IPA (11 vol). The flask was vacuum purged with nitrogen 3×.
2. The solution was heated to reflux (approximately 83° C.).
3. At reflux the bulk appeared to be in solution; however, a flocculent solid precipitated (inconsistent with the appearance of the crystalline solid). This quantity of solid was inconsistent with expectations.
4. The addition of an extra volume of IPA resulted in no apparent reduction in the amount of solid present. An aliquot of the reaction mixture was removed and the volume of IPA was doubled and the aliquot heated to reflux; however, full dissolution was not observed.
5. The reaction was cooled to ambient temperature, at this point there was two precipitates present, a sticky looking pale yellow solid and a white to off white crystalline solid.
6. To clarify the insoluble material, the volume of IPA was increased to 30 vol and heated to 60-65° C. prior to filtration. A significant amount of solid remained present, which was clarified from the solution under nitrogen.
7. The pale yellow filtrates were concentrated to approximately 11 vol. at 50-60° C. under vacuum.
8. The slurry was heated to reflux (83° C.). Full dissolution was obtained (dark yellow solution).
9. The solution was cooled to 70° C. Crystallization initiated at 72° C. and the stirring rate was reduced. The slurry was held at 70° C. for 1 h.
10. The slurry was cooled to 18 to 23° C. at approximately 10° C./h and aged for 16 h at 18-23° C.
11. The product was filtered under nitrogen and washed with IPA (2×3 vol) at 18-23° C. 4.7 g of solid was recovered from concentration of the liquors and washes.

The aqueous route performed as expected up to initial isolation. The filtrations were acceptable; however, the filter cake was sticky, difficult to paste down, and cracked heavily. The filter cake was difficult to dry and dried non-uniformly.

During the IPA crystallization of the isolated crude a quantity of insoluble solids was encountered during the crystallization. The insoluble substance has an apomorphine-related HPLC and ¹H NMR profiles, and LCMS indicates the mass ion for apomorphine. A clarification in 30 vol of IPA was included in the process to remove the solids and mitigate any affect the solids may have on the crystallization and purity of the product. The crystallization resulted in lower recovery (71% vs. expected 85-90%, the remaining mass was found in the liquors). The crystalline form by XRPD (FIG. 9) was typical of apomorphine IPA solvate, although the particle size was smaller than some previously isolated materials.

### Example 8: Non-Aqueous Salt Release for Apomorphine Hydrochloride Using 3-Amino-1-Propanol

Following a screen of organic bases and basic resins, a scalable non-aqueous salt release for apomorphine hydrochloride was developed using 3-amino-1-propanol as the organic base. The 3-amino-1-propanol hydrochloride is removed to the mother liquor on isolation of the apomorphine free base from IPA. The addition of antioxidants was investigated and it was concluded that the addition of 0.1 wt % of ascorbic acid-6-palmitate resulted in a reaction mixture with a greater stability. The product isolated via the non-aqueous route is a highly crystalline mono-IPA solvate; the isolation was developed to control the crystallization which gave control over residual 3-amino-1-propanol and ascorbic acid-6-palmitate and generated a large particle size.

In comparison with the aqueous method, the developed route results in a product which is isolated in higher overall yield, 86% vs. 75%. By contrast, if the current IPA crystallization is applied to the material derived from the aqueous route, the crystallization results in an overall process yield of 50%.

### Example 9: Solvent Screen for Additional Crystal Solvate Forms

### Batches Used for the Studies

Apomorphine free base batches: A0513-002-03, A0513-132-07, and A0530-020-01 were used for the salt investigation. Apomorphine•1*IPA batches: A0513-126-01, A0530-020-01, and A0526-010-A1 were used for the polymorph investigation.

Apomorphine free base was investigated with solvents that belonged to European Pharmacopoeia/ICH Classes 2 and 3. Twenty six crystalline or partially crystalline solids were identified; the remaining isolated solids were amorphous by XRPD. The diffraction patterns of the 26 crystalline solids were then compared and 13 of these were found to be consistent with a solid phase impurity described below that was recovered during the crystallization of the 30 g batch prepared via the aqueous route. The 12 remaining crystalline and partially crystalline solids (not including the product isolated from isopropanol, A0530-010-T1), that were not concordant with either apomorphine•1*IPA Form A or the impurity (A0486-178-A1), are believed to be novel. Characterization data of the solvents diffraction patterns and assumed temperatures of solvent release are shown in FIGS. 33-57. The solvates contained variable amounts of amorphous apomorphine that was not solvated and this disrupted the solvent.

Solvates from the Polymorph Investigation

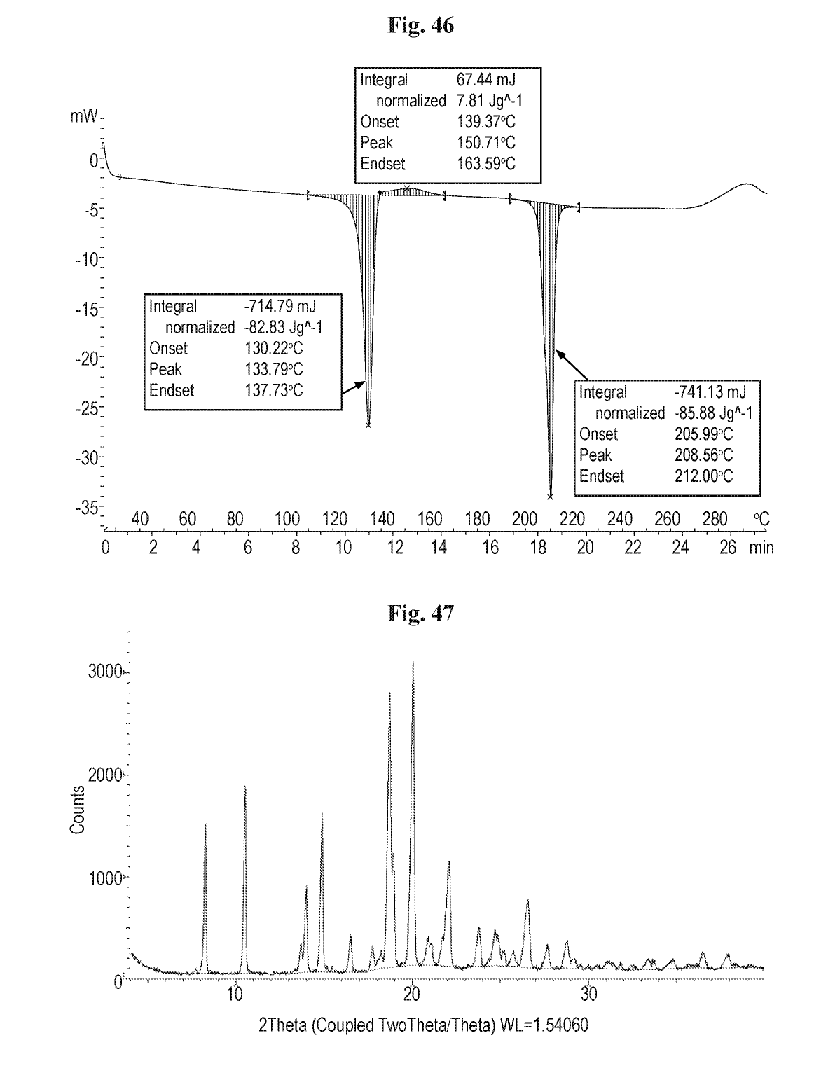

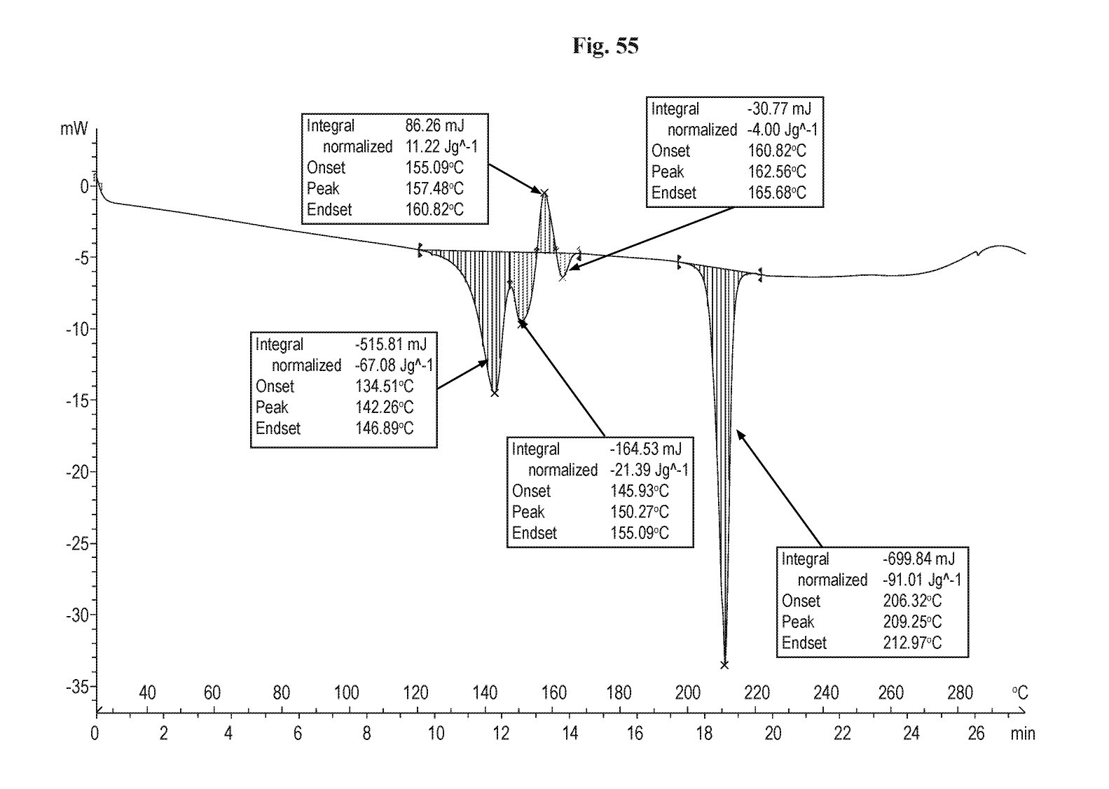

During suspension equilibrations of apomorphine•1*IPA in a variety of solvents, under aqueous and anhydrous conditions (described below), five crystalline solvates were unintentionally generated via exchange of the isopropanol:
Apomorphine•0.5*acetone (FIG. 45), onset temperature of acetone release was 130° C. (FIG. 46)
Apomorphine•1.0*TBME (FIG. 47), onset temperature of TBME release was 102° C. (FIG. 49)
Apomorphine•0.2*isopropyl benzene•0.5*IPA (FIG. 50), onset temperature of solvent releases was 74° C. (FIG. 52), de-solvation events were complex
Apomorphine•0.5*ethanol (FIG. 53), onset temperature of ethanol release was 135° C. (FIG. 55)
Apomorphine•0.5*THF (FIG. 56), onset temperature of THF release was 126° C. (FIG. 57)

The main diffraction peaks of the ethanol and TBME solvates corresponded to those first identified in the solvate screen. Whilst the main diffraction peaks of the acetone and THF solvates were markedly different from those identified in the solvate screen these differences were attributed to possible polymorphism.

### Example 10: Investigation of the Impurity

An impurity was first isolated during the 30 g crystallization of apomorphine•1*IPA from isopropanol, prepared via the aqueous route. The impurity was isolated from 13 of the 26 crystalline solids generated during the solvate screen. The identification of this impurity was attempted.

Analytical data obtained from the impurity (A0486-178-A1) was compared to the corresponding analysis performed on the recrystallized demonstration batch of apomorphine•1*IPA (A0526-010-A1). The findings from this investigation are summarized below:
The solid phases were different by XRPD and DSC analyses.
Both samples contained an equal number of protons to apomorphine; however, protons local to the nitrogen were shifted (by ¹H NMR).
Both samples contained an equal number of carbons to apomorphine that exhibited the same sp-hybridization (by ¹³C NMR & DEPT); excluding the possibility of carbonic acid adducts or a cyclic carbonate condensed at the 10, 11-diol positions.
Both samples had mass 268 [M+H], by LCMS.
Retention times by MET/CR/2498 were the same.
The pH of an aqueous solution of the impurity was 7, indicating that the impurity was not sodium phenoxide(s) at the 10, 11-diol positions.
No effervescence was observed (i.e., gas release e.g., CO₂) during the preparation of the aqueous solution of the impurity.
FTIR to determine if R₃N⁺—O⁻ (1310 to 1200 cm⁻¹) was present in the impurity was inconclusive; however, the stretch at 3496.4 cm⁻¹ corresponding to —OH was present in apomorphine•1*IPA and was absent from the impurity.
Wet chemistry tests for the presence of residual NaCl (by AgNO₃) and Na₂S₂O₅ (by KMnO₄) were inconclusive due to reaction with the apomorphine impurity.
Solubility of the impurity in methanol and IPA was lower than that of apomorphine•1*IPA.
The impurity is unlikely to be a CO₂ adduct or ester or sodium salt.
   Treatment with CO₂

To establish if treatment of apomorphine•1*IPA with carbon dioxide under aqueous conditions resulted in the formation of an ionic carbon dioxide adduct (apomorphine hydrogen carbonate or disproportionated into apomorphine carbonate) or generated a formal covalent cyclic carbonate under anhydrous conditions (Scheme 6), the following experiments were performed:

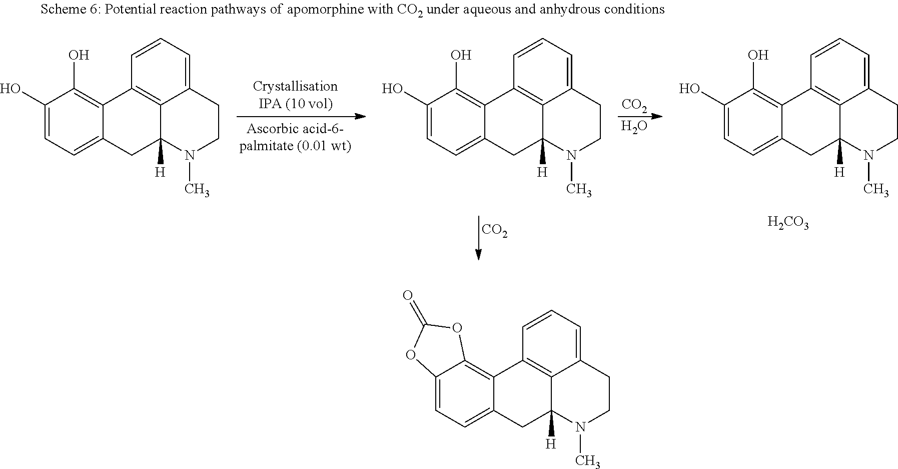

### Low Temperature Anhydrous CO₂ Treatment

### A0505-120-A1

A solution of apomorphine•1*IPA in isopropanol (50.0 vol) at 50-55° C. was quenched by a single pellet of dry ice, the product was isolated by filtration (65% theoretical) and was consistent with apomorphine•1*IPA, Form A by XRPD. The structure was concordant with apomorphine by ¹H NMR and contained one equivalent of isopropanol.

### Ambient Temperature Aqueous CO₂ Treatment

### A0505-120-B1

A suspension of apomorphine•1*IPA, Form A, was stirred in IPA/water (10.0/9.0, v/v, 11.0 vol) at 18-23° C., under CO₂ at balloon pressure for 24 h, the product was consistent with apomorphine•1*IPA by XRPD. The structure was concordant with apomorphine by ¹H NMR and contained one equivalent of isopropanol.

### Example 11: Polymorph

Two principal forms of apomorphine were incorporated into the polymorph investigation; the form generated by crystallization from isopropanol and designated as Form A (Table 9) and the form derived by precipitation from water (consistent with amorphous material). Apomorphine•1*IPA with not less than 16.5% w/w and not more than 20.2% w/w isopropanol content by GC-HS, MET/CR/2497 was preferred.

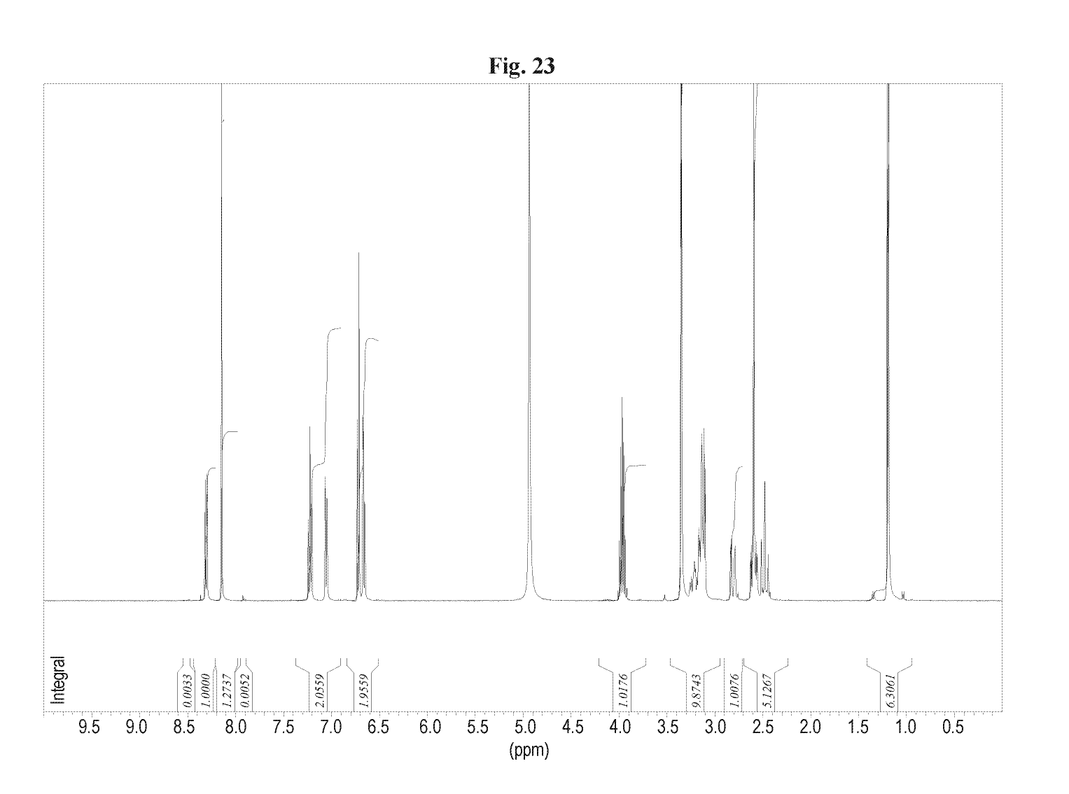

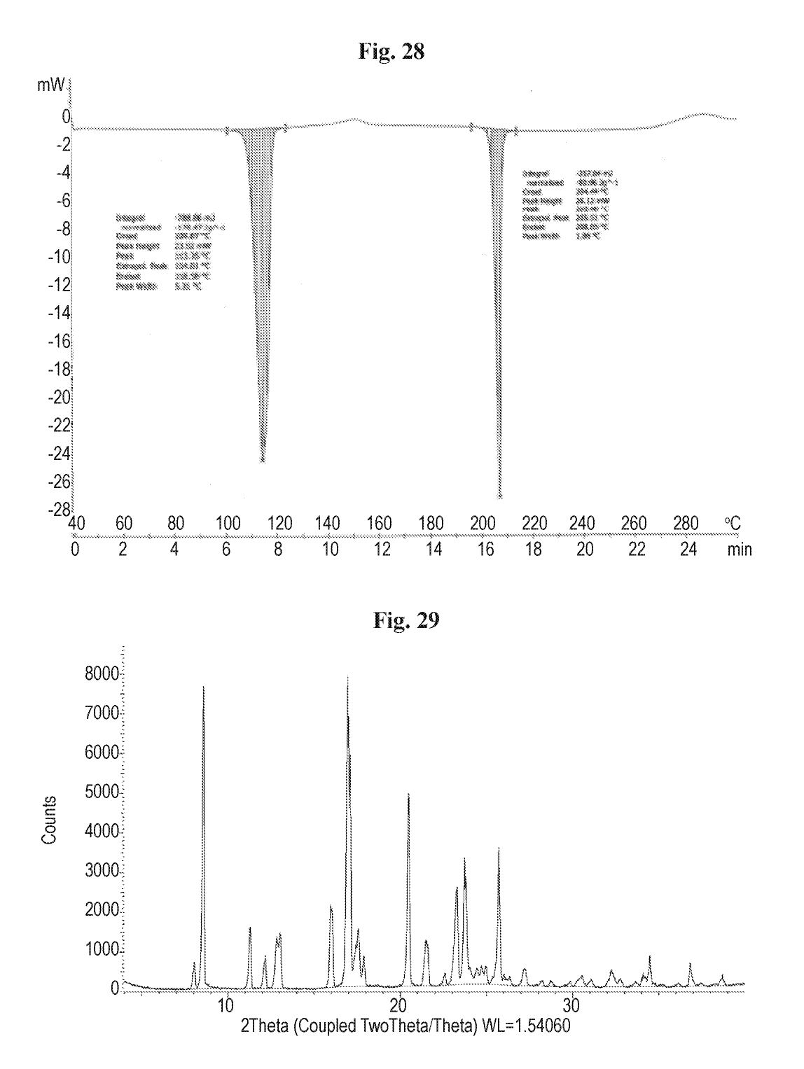

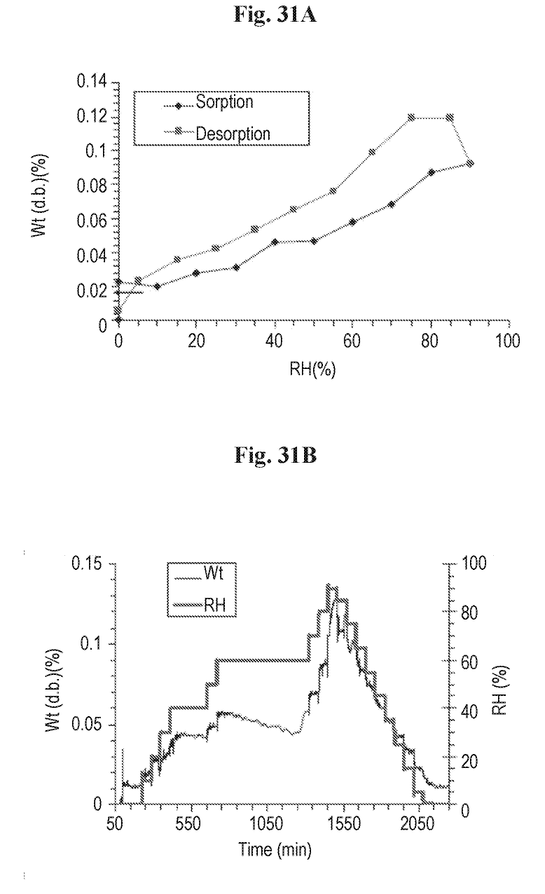

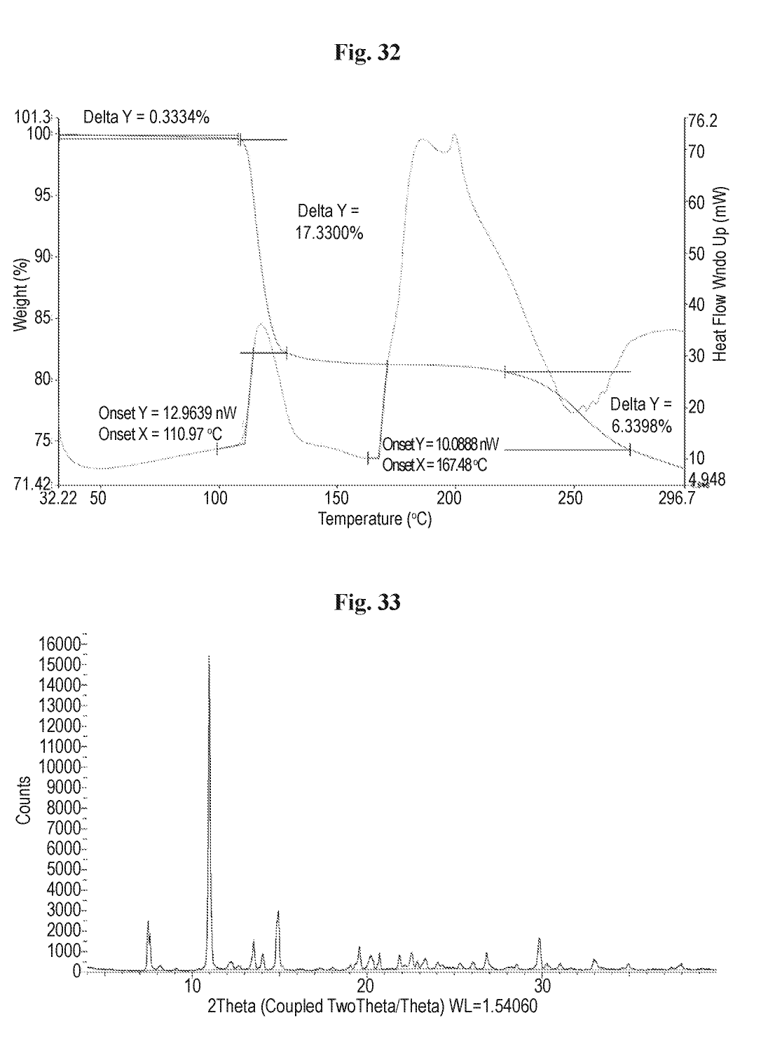

TABLE 9
Characterization of the IPA solvate form

| Provenance | Apomorphine•1*IPA (Form A) |
|---|---|
| Apomorphine•1*IPA | Batch: A0526-010-A1 |
| (batch A0526-004-B1 | Solvated molecular formula: C₂₀H₂₅NO₃ |
| perpared from | Solvated Mᵣ: 327.43 |
| apomorphine | Isolation solvent class: ICH 3 (IPA) |
| hydrochloride | ¹H NMR: conformed to molecular structure |
| batch JM 11-00645) | (FIG. 23) |
| | ¹H NMR assay^{a} (acquired against TCNB as |
| | the internal standard with an extended relaxation |
| | of 20 s): 100% w/w on anhydrous solvent free |
| | basis |
| | IPA content: 18.5% w/w (by GC-HS), 19.2% w/w |
| | by ¹H NMR |
| | Apomorphine to IPA ratio^{a}: 1.0 to 1.0 |
| | XRPD: 7.98°, 8.49°, 11.17°, 12.03°, 12.69°, |
| | 12.88°, 15.97°, 16.83°, 17.00°, 17.36°, 17.72°, |
| | 20.31°, 21.39°, 22.43°, 23.02°, 23.71°, 24.09°, |
| | 24.85°, 25.60°, 27.04°, 30.35°, 32.18°, 34.38° |
| | 2Theta (FIG. 29) |
| | DSC: onset of isopropanol release 109.9° C. |
| | (endotherm, sharp), probable melt event onset: |
| | 204.4° C. followed by decomposition (FIG. 28) |
| | Thermal microscopy: characterised by solvent |
| | release, crystallization, melt and decomposition |
| | Water content: <0.1% w/w (KF titration, |
| | Aquamicron AX) |
| | Equilibrium humidity: absorbate stable at 18-23° |
| | C. over 144 h |
| | GVS: +0.1% w/w up to 90% RH, categorised |
| | (PHEur.) as non-hygroscopic (FIG. 31A) |
| | STA (TGA): weight loss transition of 17.3% w/w |
| | was observed at approximately 111° C. |
| | (FIG. 32) |
| | Specific optical rotation: −46.7° [α]_{D} |
| | (c = 1.12 in 0.1N HCl) |

The salt release of apomorphine hydrochloride in the presence of antioxidant ascorbic acid-6-palmitate is performed in IPA by treatment with 3-amino-1-propanol. Apomorphine•1*IPA then crystallizes from solution as prisms and 3-amino-1-propanol hydrochloride remains in solution. A second crystallization of apomorphine•1*IPA from IPA is then performed to upgrade the appearance of the material.

The salt release of apomorphine hydrochloride in the presence of antioxidant sodium metabisulphite is performed under aqueous conditions by treatment with sodium hydrogen carbonate. Amorphous apomorphine then precipitates from solution.

The polymorph investigation consisted of techniques to induce stable forms such as suspension equilibrations and crystallizations in conjunction with techniques intended to promote kinetic forms e.g., ballistic cooling and co-solvent precipitations, etc.

### Thermal Evaluations

### Analysis by DSC

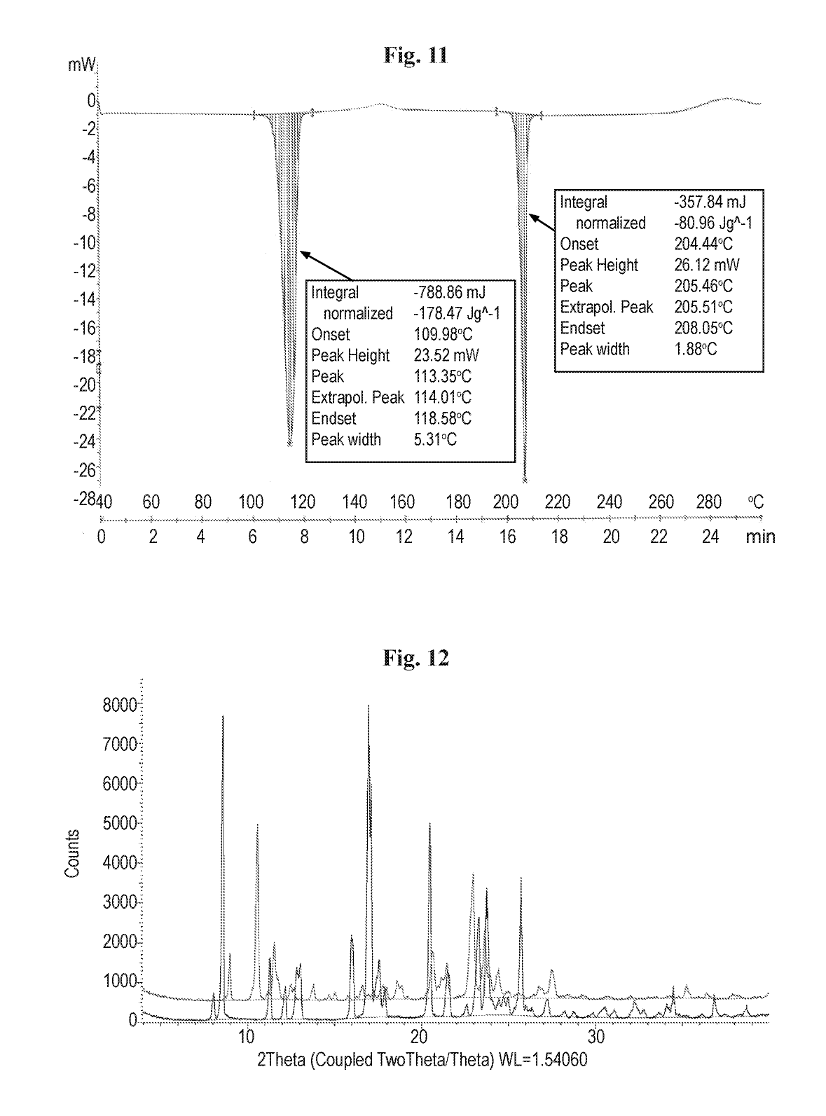

The DSC of apomorphine•1*IPA (Form A) contained a large endothermic event with onset 110° C. (FIG. 11); this event was attributed to the thermal release of IPA.

To confirm that apomorphine•1*IPA (Form A) and the related desolvated form (apomorphine•0*IPA) were not isostructural by XRPD, a specimen of Form A IPA solvate (FIG. 11) was thermally desolvated by heating from 20 to 140° C. to reach the first endothermic event that corresponded to isopropanol release.

The freshly dehydrated specimen was then expressed from the crucible under nitrogen and analyzed by ¹H NMR and XRPD (FIG. 12).

The dehydrated specimen that had undergone thermal release of IPA (A0526-010-A1 140° C.) was not consistent with authentic Form A IPA solvate (A0526-010-A1), indicating that desolvation had altered the crystal structure. Absence of the IPA was confirmed by ¹H NMR analysis.

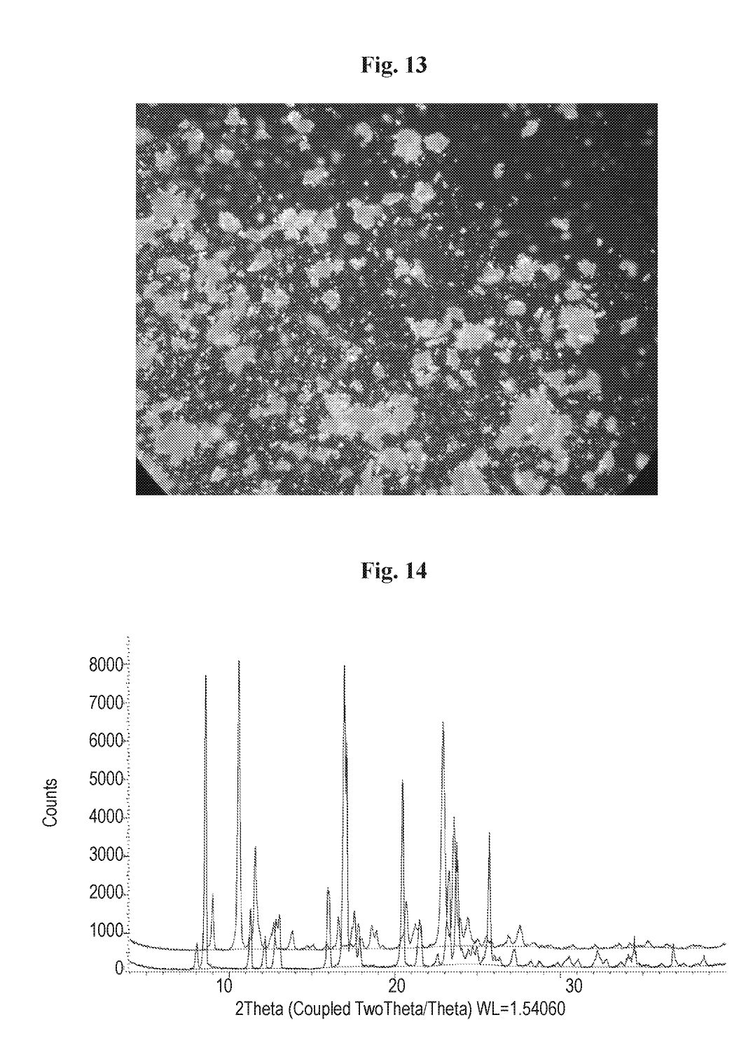

To gain better understanding of the small exothermic event present on the DSC (approximately 140-160° C., FIG. 11), a specimen was heated up to 180° C., cooled to 20° C. under nitrogen, and analyzed by optical microscopy (irregular morphology, little birefringence, FIG. 13) and XRPD (FIG. 14). The diffraction pattern was consistent with solvent released apomorphine.

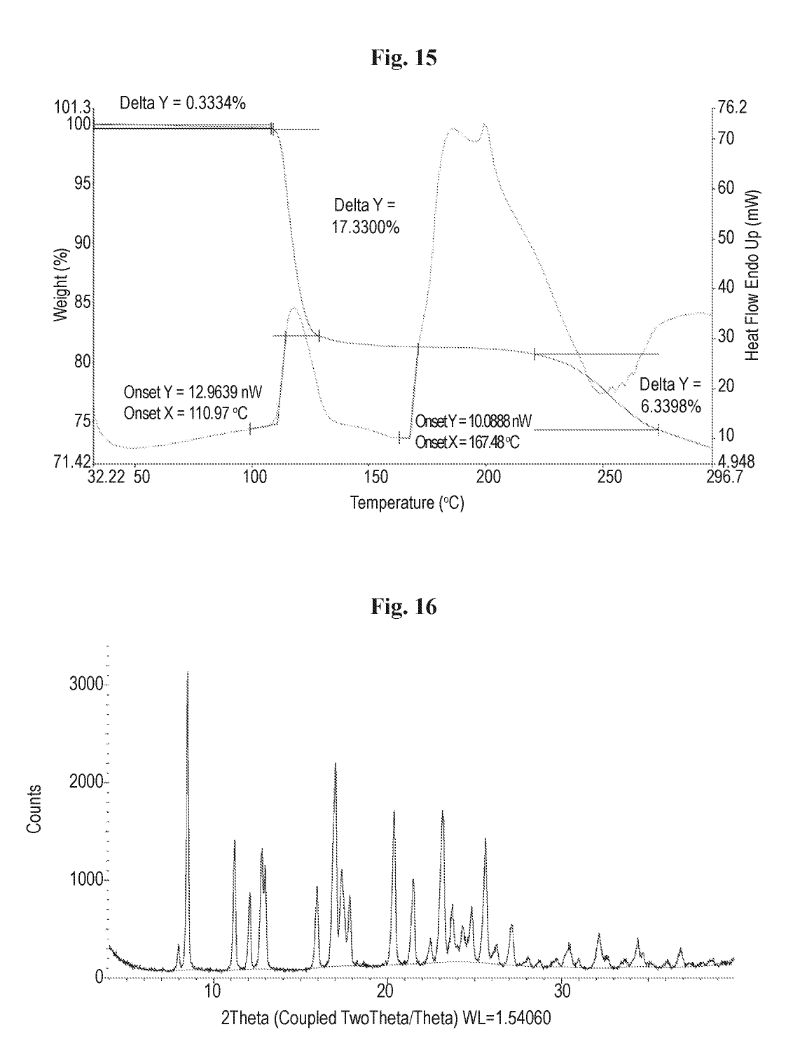

FIG. 15 shows the TGA analysis of apomorphine•1*IPA (Form A). A weight loss transition of 17.3% w/w was observed at approximately 111° C.; this corresponded to IPA release and supported the observations made by DSC. Weight loss by degradation was observed from approximately 250° C. and the DTA trace (hashed line) suggested a single melt event with onset of 270-275° C.

### Thermal Microscopy Performed Under Air

The endotherm observed by DSC with onset 110° C. corresponded to release of a liquid from the crystal by thermal microscopy, attributed to IPA desolvation of apomorphine•1*IPA (FIG. 11). Release of IPA was captured by thermal microscopy (FIG. 65), which showed obliteration of the crystals and loss of birefringence under cross polarized light. The small exotherm observed by DSC with onset approximately 150° C. appeared to correspond to crystallization of a small quantity of amorphous material (FIG. 67). Extensive decomposition was observed in the heat up to the second endotherm (FIG. 69).

### Thermal Microscopy Performed Under Nitrogen

The thermal microscopy analysis was repeated under a fast stream of nitrogen. The cooling effect of the nitrogen affected the temperature readings. Release of IPA was observed at approximately 134° C. A transition occurred >200° C. and needles began to grow. The needles proliferated until they melted and had cleared by 260° C. The chemical identity of the specimen at this temperature was not known. When the melt was exposed to the air, rapid discoloration occurred.

### High Pressure Evaluations

Specimens of amorphous apomorphine (5 mg, A0530-020-01) and apomorphine•1*IPA (Form A), (A0526-010-A1) were compressed under a pressure of 10 tones (10 t, 10×1000 kilograms), under nitrogen at 18-23° C. and maintained under these conditions for 1 h. After treatment all of the specimens became ‘glassy’ in appearance. XRPD analysis of the recovered specimens showed an increased amorphous content (A0526-010-A1) and a corresponding loss of resolution of the main reflections (Table 10).

TABLE 10
Results from the high pressure study

| Reference | Input form | Output form |
|---|---|---|
| A0530-020-01 | Amorphous | Amorphous |
| A0526-010-A1 | Form A | Form A, slightly disordered |

### Suspension Equilibrations

Suspension equilibration under anhydrous or aqueous conditions is a thermodynamic technique. The technique was applied to apomorphine•1*IPA (Form A) to determine if the phase evolved into a more stable modification.

### Anhydrous Suspension Equilibrations

Sixteen portions of apomorphine•1*IPA (approximately 100 mg, 1.0 wt.), antioxidant (0.01 wt.) and the appropriate solvents (1000-2000 μl, 10.0-20 vol) were charged to 16 separate vessels and stirred for 5-10 days at 40-45° C. If the majority of the solid dissolved, the solution was concentrated by approximately one half under nitrogen and if a suspension was generated then equilibration was resumed. If no such suspension was generated then the experiment was repeated using an equal volume of an inert diluent such as n-heptane, if the majority of the solids still dissolved under these conditions then the experiment was abandoned.

After this time the products were isolated by filtration, washed with recycled maturation solvent, dried under a stream of nitrogen at 18-23° C. and analyzed by XRPD for evidence of alternative crystalline forms and their compositions determined by ¹H NMR.

No alternative phases to the apomorphine•1*IPA prepared in the demonstration batch were identified. Typically, IPA was removed or partially exchanged from apomorphine•1*IPA by the suspension equilibration solvent. Suspension equilibrations of apomorphine•1*IPA in IPA under anhydrous (A0505-080-N1) and aqueous (A0505-090-N1) conditions generated materials that were consistent with the authentic starting material by XRPD.

Suspension equilibrations in anhydrous n-heptane (A0505-080-J1) generated a mixture of unchanged apomorphine•1*IPA solvate and a new phase (believed to be crystalline apomorphine free base). Treatment of apomorphine•1*IPA with cumene (isopropylbenzene) generated mixed solvates (A0505-080-E1 and A0505-090-E1) that were isostructural with the input material; the solvent stoichiometries were disrupted by the presence of amorphous free base. Five crystalline solvates were obtained by exchange of the solvated isopropanol and were characterized by ¹H NMR, XRPD and DSC:
Apomorphine•0.5*acetone (A0505-080-A2, FIG. 45)
Apomorphine•0.5*IPA•0.2*cumene (A0505-080-E1, FIG. 50, and A0505-090-E1, FIG. 51)
Apomorphine•1.0*TBME (A0505-080-D1, FIG. 47, and A0505-090-D1, FIG. 48)
Apomorphine•0.5*EtOH (A0505-080-G1, FIG. 53, and A0505-090-G1, FIG. 54)
Apomorphine•0.5*THF (A0505-080-O2, FIG. 56)
Diffraction patterns of the solvates isolated from anhydrous or aqueous conditions were the same

### Shock Cooled Crystallizations

The effect that different rates of cooling had on solutions of apomorphine in IPA were examined to show that crystallization promoted by rapid cooling does not alter the physical phase of the isolated apomorphine•1*IPA product. The experiments are described below and the results are summarized in Table 11.

### Cooled to the Cloud Point and Aged Approximately 16-20 h (A0505-124-A1, Control Experiment)

Apomorphine free base (150 mg, 1.0 wt), ascorbic acid-6-palmitate (1.5 mg, 0.01 wt) and IPA (1650 μl, 11.0 vol) were stirred at 18-23° C. The suspension was sonicated to degas and placed under a flow of nitrogen at 18-23° C. The suspension was heated to approximately 80-85° C., to effect dissolution. The solution was stirred for 15-20 minutes, cooled until the cloud point was observed approximately 70° C. and aged at the cloud point temperature over 16-20 h. The suspension was filtered, washed with IPA (2×450 μl, 2×3 vol) and dried under a flow of nitrogen at 18-23° C. for at least 30 min.

### Shock Cooled to 0° C. and Aged 1-2 h at the Same Temperature (A0505-124-B1, Estimated Cooling Rate: Approximately −85° C./s or −5100° C./Min)

The experiment was repeated as above (A0505-124-A1) except the hot solution at 80-85° C. was plunged into ice water at 0° C. The suspension was aged at this temperature for 1-2 h, filtered, washed with isopropanol (2×450 μl, 2×3 vol) and dried under a flow of nitrogen at 18-23° C. for at least 30 min.

### Ballistic Cooled to −78° C. and Isolated Immediately (A0505-124-C1, Estimated Cooling Rate: Approximately −163° C./s or −9780° C./Min)

The experiment was repeated according to A0505-124-A1, except the hot solution at 80-85° C. was plunged into carbon/dioxide acetone at −78° C. The suspension was filtered immediately, washed with IPA (2×450 μl, 2×3 vol) and dried under a flow of nitrogen at 18-23° C. for at least 30 min.

The isolated products were consistent with apomorphine•1*IPA (Form A) and the yields ranged from 76-81% (theoretical).

The crystallization procedure was able to tolerate natural and accelerated rates of cooling and still generate apomorphine•1*IPA as the isolated product (Table 11). The isolated samples exhibited good crystalline diffraction patterns (e.g., FIG. 16); however, there was some evidence of amorphisation and broadening of the reflections but this was not significant when compared to the demonstration batch.

TABLE 11
Crystallizations of apomorphine from IPA (boiling point 82° C.; ICH = 3; 11 vol;
dissolution temperature = 90° C.), promoted by different rates of cooling

| | | Ascorbid- | | | | | | |
|---|---|---|---|---|---|---|---|---|
| | | 6- | | | | | | |
| | | palmitate | | | | | | Molar ratio |
| | | input | Apomorphine | | | | % w/w of | of IPA to |
| Reference | Apo | (mg) | input (mg) | Cooling | Yields* | XRPDs | solvent | apomorphine |
| A0505- | apomorphine•1*IPA | 1.8 | 149.7 | Natural | 81% | Form A | 18.7% | 1.0 |
| 124-A1 | (A0526-010-A1) | | | cooling | | | | |
| A0505- | | 1.7 | 149 | Crash | 79% | Form A | 18.6% | 1.0 |
| 124-B1 | | | | cooled | | | | |
| | | | | to 0° C. | | | | |
| A0505- | | 1.7 | 151.8 | Balistic | 76% | Form A | 20.3% | 1.1 |
| 124-C1 | | | | cooled | | | | |
| | | | | to −78° C. | | | | |

*Theoretical

### Seeded Crystallizations

The effect of seeding solutions of apomorphine in IPA was examined to show that Form A is still isolated even after nucleation by alternative physical forms of apomorphine.

### General Procedure A0505-128-A1 to C1

Apomorphine free base (150 mg, 1.0 wt), ascorbic acid-6-palmitate (1.5 mg, 0.01 wt) and isopropanol (1650 μl, 11.0 vol) were stirred at 18-23° C. The suspension was sonicated to degas and placed under a flow of nitrogen at 18-23° C. The suspension was heated to approximately 80-85° C. to effect dissolution. The solution was stirred for 15-20 minutes, cooled until the cloud point was observed approximately 70° C. The temperature was increased by approximately 5° C. to re-dissolve the solids and apomorphine seeds (3.0 mg, 2% w/w) were charged to the solution. The fates of the seeds were observed. The suspension was aged at this temperature for 1-2 h and cooled to 18-23° C. The suspension was filtered, washed with IPA (2×450 μl, 2×3 vol) and dried under a flow of nitrogen at 18-23° C. for at least 30 min.

Seeds Used:

Apomorphine free base (A0530-020-01, generated via the aqueous method), apomorphine•1*IPA (A0505-096-A1, generated via evaporation from IPA and exhibited erroneous reflections by XRPD at 2Theta 24.2° and 20.7°) and apomorphine hydrochloride.

The isolated products were consistent with apomorphine•1*IPA (Form A) and the yields ranged from 75-82% (theoretical).

The crystallization procedure was able to tolerate seeding at 2% w/w by different forms and phases of apomorphine and still generate apomorphine•1*IPA (Table 12).

### Evaporation Crystallizations

These experiments were performed to determine if metastable kinetic forms of apomorphine•1*IPA were generated by evaporation of the corresponding solution in IPA.

### Slow Evaporation at 40° C. Under Standard Pressure (A0505-096-A1)

Apomorphine free base (150 mg, 1.0 wt), ascorbic acid-6-palmitate (1.5 mg, 0.01 wt) and IPA (150000, 100.0 vol) were charged to a capped vessel and stirred at 18-23° C. under nitrogen. The slurry was heated to approximately 80-85° C. until full dissolution had taken place. The solution was cooled to 40° C. and left to stand under a very slow stream of nitrogen whilst evaporated to dryness over 18 h to give a pale brown solid.

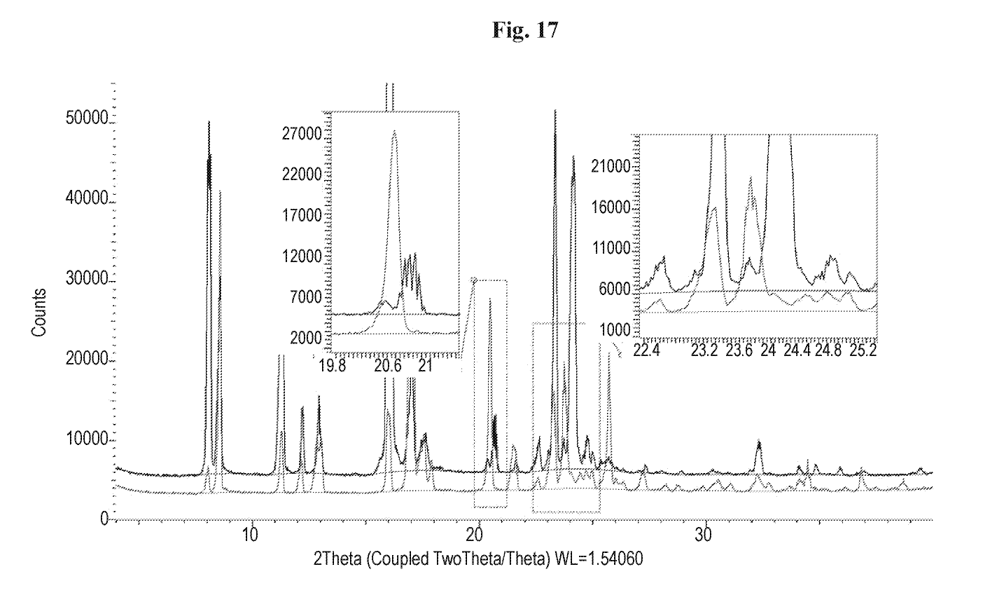

The pale brown solid was recovered in quantitative yield and contained isopropanol (17.3% w/w) that corresponded to 1.0 to 0.91 apomorphine:IPA stoichiometry (by ¹H NMR). Some differences were evident by XRPD at 2Theta 24.2° and 20.7° (FIG. 17). DSC Analysis (FIG. 18) showed ill-defined and broad, solvent release and melt events compared to the recrystallized demonstration batch (request for analysis 95391, sample A0526-010-A1). The fate of this material after suspension equilibration in IPA was determined to confirm conversion into Form A, should an analogous form arise during manufacture.

TABLE 12
Crystallizations of apomorphine from IPA (boiling point 82° C.; ICH = 3; 11 vol;
dissolution temperature = 90° C.), seeded with alternative salts and phases

| | | Ascorbid- | | | | | | |
|---|---|---|---|---|---|---|---|---|
| | | 6- | | | | | % w/w | Molar ratio |
| | | palmitate | Apomorphine | | | | of | of IPA to |
| Reference | Apo | input (mg) | input (mg) | Seeding | Yields* | XRPDs | solvent | apomorphine |
| A0505-128-A1 | A0526-010-A1 | 1.8 | 150.7 | Seeded with | 81% | Form A | 18.4% | 1.0 |
| | | | | A0530-020-01 | | | | |
| | | | | (free base via | | | | |
| | | | | aqueous | | | | |
| | | | | method) | | | | |
| A0505-128-B1 | | 1.5 | 151.7 | Seeded with | 82% | Form A | 18.4% | 1.0 |
| | | | | A0505-096-A1 | | | | |
| | | | | via evaporative | | | | |
| | | | | crystallization | | | | |
| A0505-128-C1 | | 1.9 | 151.4 | Seeded with | 75% | Form A | 18.7% | 1.0 |
| | | | | apomorphine | | | | |
| | | | | hydrochloride | | | | |

*Theoretical

### Suspension Equilibration of Evaporative Crystallization Product (A0505-106-A1)

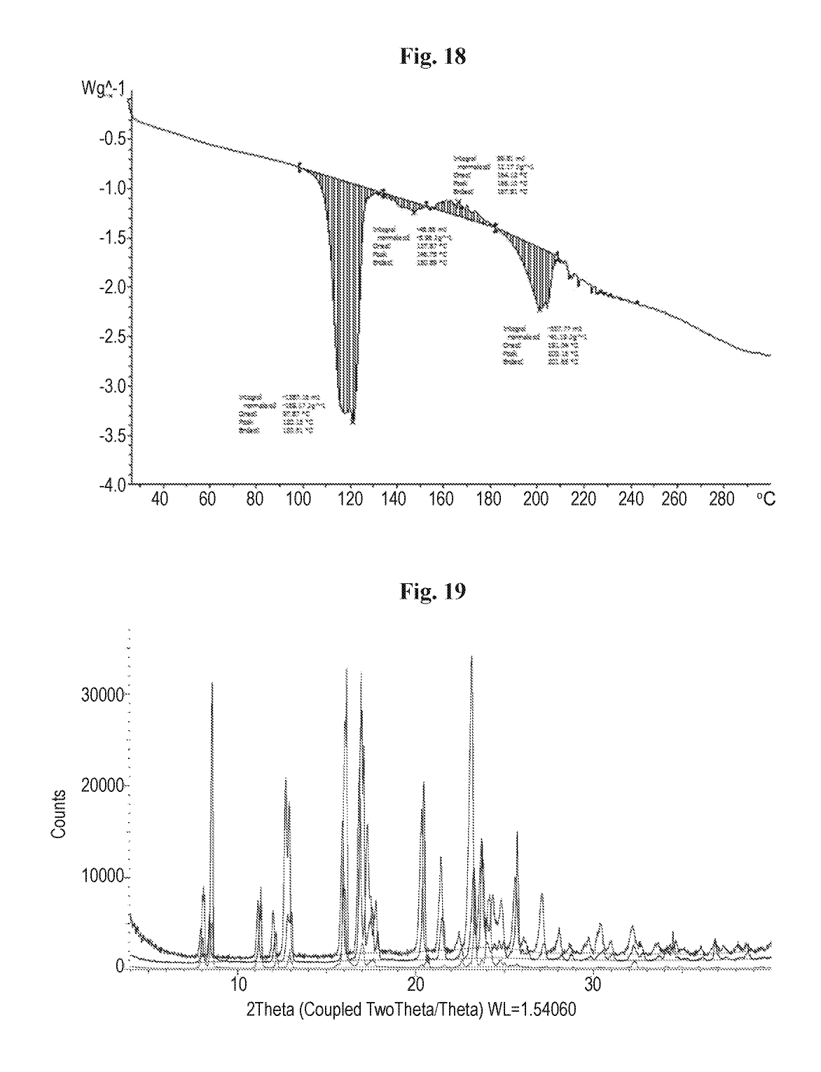

The product obtained from experiment A0505-096-A1 (100 mg) was stirred at 40 to 45° C. for 3 days under nitrogen, filtered, washed with recycled liquors to give 78 mg (78% theoretical) as an off-white solid. XRPD analysis (FIG. 19) confirmed conversion into apomorphine•1*IPA Form A. IPA content was 18.3% w/w by ¹H NMR and DSC of solvent release and melting were consistent with Form A.

### Slow Evaporated Under Ambient Temperature and Pressure (A0505-096-B1)

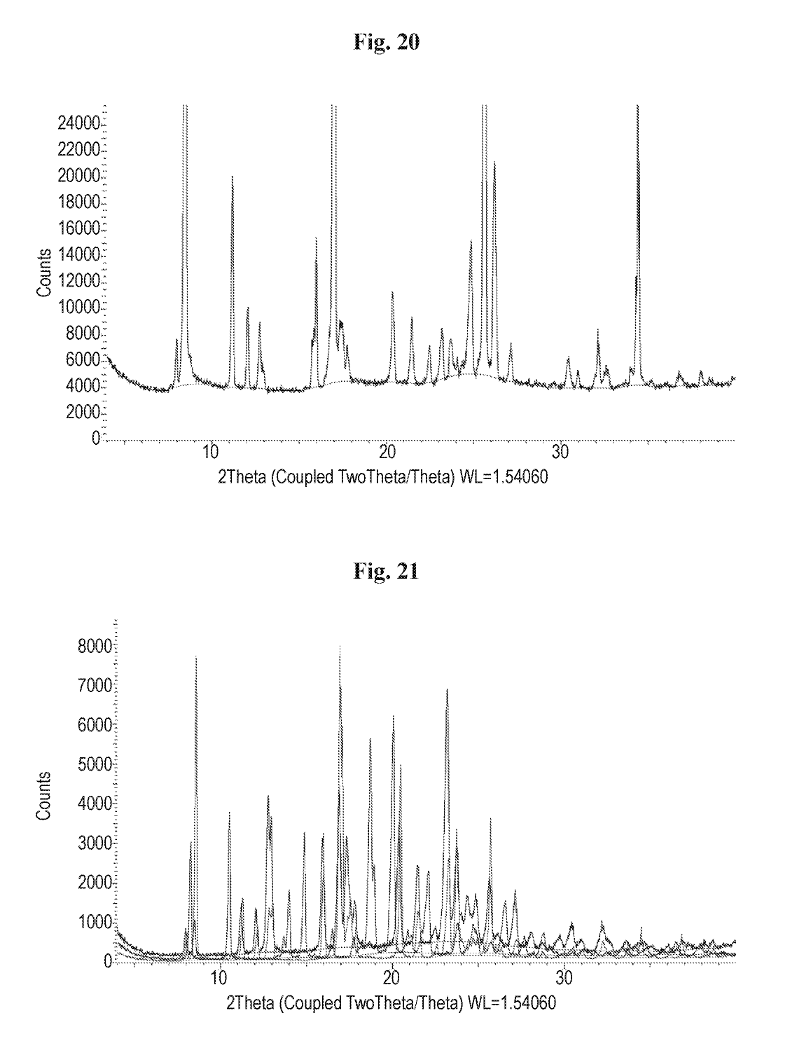

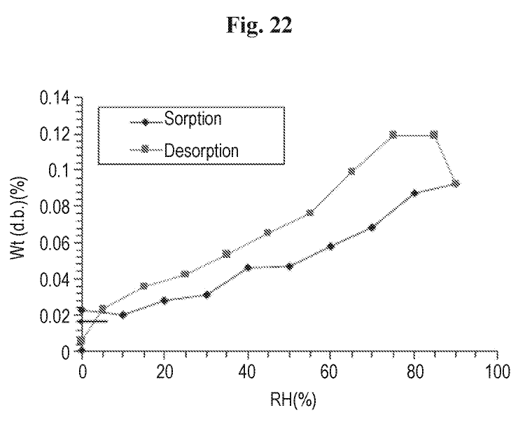

As above, except the solution was left to stand under a very slow stream of nitrogen whilst cooling to 18-23° C. until evaporated to dryness. Pale brown solid was recovered in quantitative yield. The diffraction pattern (FIG. 20) was consistent with authentic apomorphine•1.0*IPA Form A (A0526-010-A1) and the isopropanol content was 17.3% w/w, corresponding to 1.0 to 0.93 apomorphine:isopropanol stoichiometry (¹H NMR).

### Co-Solvent Crystallizations

The purpose of these experiments were to determine if metastable kinetic forms of apomorphine•1*IPA were generated by precipitation promoted by co-solvent addition to an IPA solution.

### General Procedure A0505-098-A1 to P1

Sixteen portions of apomorphine free base (150 mg, 1.0 wt.) and ascorbic acid 6-palmitate (1.5 mg, 0.01 wt) were charged to 16 separate vessels. IPA (15000, 10.0 vol) was charged to each vessel, the vessels were capped and heated to 80-85° C. to affect full dissolution. The relevant solvent (15000, 10.0 vol) was then charged to each of the hot solutions. The mixtures were left to cool to 18-23° C., with stirring over 16-20 h and the products were isolated by filtration under nitrogen (Table 13).

No alternative phases to the apomorphine•1*IPA (Form A), prepared in the demonstration batch were identified. However, two product subsets were identified. The first set consisted of mixed occupancy solvates, isostructural with apomorphine•1*IPA (Form A) and the second set consisted of low yields of the impurity, consistent with the clarification residue (A0486-178-A1) recovered from the 30 g batch that used free base apomorphine derived from the aqueous route (See Example 10). An explanation for the presence of this impurity was that the starting material (A0530-020-01) used for the investigation was apomorphine free base that was derived from the aqueous route.

Mixed occupancy solvates were generated when IPA was partially exchanged from apomorphine•1*IPA by the precipitant solvent, except in the cases of diethyl ether and n-heptane (A0505-098-I1 and A0505-098-J1, Table 13) where no exchange took place, presumably because of the poor solubility of the solvate in these solvents.

Evidently, apomorphine•1*IPA Form A can alter its solvated composition if certain solvents or water are present at high enough activities to effect displacement. Partial replacement of IPA by the competitive solvent avoids disruption to the crystal phase by XRPD. However, solvates or hemi-solvates obtained from the suspension equilibration screen, in which complete replacement of the IPA occurred, resulted in new diffraction patterns by XRPD.

Precipitation of apomorphine•1*IPA from IPA, by isopropanol generated a product (A0505-098-N1, Table 13) that was consistent with the authentic starting material by XRPD. This supports the assumption that undesirable forms of apomorphine•1*IPA will not be generated by uncontrolled crystallization during manufacture, or if they are, they readily convert to or revert back into Form A.

TABLE 13
Results from the co-solvent crystallization screen (apomorphine free base, input:
A0530-020-01; co-solvent 1: 2-propanol, 10.0vol; equilibration temperature: 18-23° C.)

| | | | | Isolated under N₂ | | | |
|---|---|---|---|---|---|---|---|
| | Co-solvent | Boiling | | Isolated | | | Solvents |
| Reference | 2 (10vol) | point (° C.) | ICH | (mg) | Yields* | XRPDs | (% w/w) |
| A0505-098-A1 | Acetone | 56 | 3 | 15.3 | 10% | Impurity | IPA 4.0%, |
| | | | | | | | acetone 0.4% |
| A0505-098-B1 | Anisole | 154 | 3 | 15.0 | 10% | Impurity | IPA 0.4%, |
| | | | | | | | anisole 4.2% |
| A0505-098-C1 | 1-butanol | 118 | 3 | 13.8 | 9% | Impurity | IPA 0.8%, |
| | | | | | | | 1-BuOH 4.5% |
| A0505-098-D1 | TBME | 55 | 3 | 96.6 | 65% | Isostructural | IPA 12.7%, |
| | | | | | | with Form A | TBME 0.8% |
| A0505-098-E1 | Cumene | 152 | 3 | 91.6 | 61% | Isostructural | IPA 12.6%, |
| | | | | | | with Form A | cumene 1.7% |
| A0505-098-F1 | DCM | 40 | 2 | 16.7 | 11% | Impurity | IPA 3.0%, |
| | | | | | | | DCM 0.2% |
| A0505-098-G1 | Ethanol | 78 | 3 | 64.5 | 43% | Impurity | IPA 7.3%, |
| | | | | | | | EtOH 0.6% |
| A0505-098-H1 | Ethyl | 75 | 3 | 11.5 | 8% | Impurity | IPA 3.1%, |
| | acetate | | | | | | EtOAc 0.3% |
| A0505-098-I1 | Ethyl ether | 35 | 3 | 97.6 | 65% | Isostructural | IPA 15.3%, |
| | | | | | | with Form A | EtO₂ nd |
| A0505-098-J1 | n-heptane | 98 | 3 | 133.9 | 89% | Isostructural | IPA 15.2%, |
| | | | | | | with Form A | n-heptane 0.3% |
| A0505-098-K1 | Isopropyl | 87 | 3 | 25.2 | 17% | Impurity | IPA 4.5%, |
| | acetate | | | | | | iso-PrOAc 2.4% |
| A0505-098- | MEK | 80 | 3 | 14.4 | 10% | Impurity | IPA 1.7%, |
| L1 | | | | | | | MEK 0.7% |
| A0505-098- | Methanol | 65 | 2 | 19.8 | 13% | Impurity | IPA 4.3%, |
| M1 | | | | | | | MeOH nd |
| A0505-098- | 2-Propanol | 83 | 3 | 110.0 | 73% | Isostructural | IPA 16.1%, |
| N1 | | | | | | with Form A | |
| A0505-098- | THF | 66 | 2 | 29.2 | 19% | Impurity | IPA 3.6%, |
| O1 | | | | | | | THF 1.4% |
| A0505-098-P1 | Toluene | 111 | 2 | 78.1 | 52% | Isostructural | IPA 14.7%, |
| | | | | | | with Form A | Toluene 1.3% |

*Theoretical;
nd—not detected.

### Competitive Suspension Equilibrations

Four suspension equilibrations (A0505-116-A1 to D1) were performed to include different combinations of input forms (Table 14). The mixtures were stirred in IPA (10.0 vol) at 45-50° C. for 6 days. The resulting solids were isolated by filtration, washed with recycled maturation solvent, dried under a stream of nitrogen at 18-23° C. and analyzed by XRPD. The diffraction patterns and isopropanol contents of the products from the suspension equilibrations were consistent with authentic apomorphine•1*IPA Form A.

TABLE 14
Results from the competitive suspension inter-conversions

| | | | | Total | Outputs | | | | |
|---|---|---|---|---|---|---|---|---|---|
| | Inputs (mg)** | | | apomorphine | Isolated | | | % w/w of | Apomorphine |
| Reference | 1 | 2 | 3 | input | (mg) | Yields* | XRPDs | solvent | to IPa content |
| A0505-116-A1 | 100.8 | — | — | 100.8 | 69.7 | 69% | Form A | 18.6 | 1.0 to 1.0 |
| A0505-116-B1 | 68.1 | 49.1 | — | 117.2 | 93.4 | 80% | Form A | 18.3 | 1.0 to 1.0 |
| A0505-116-C1 | 71.2 | — | 32.7 | 103.9 | 73.2 | 70% | Form A | 18.8 | 1.0 to 1.0 |
| A0505-116-D1 | 39 | 39.6 | 34.2 | 112.8 | 89.2 | 79% | Form A | 17.7 | 1.0 to 0.9 |

*Theoretical
**1 - Apomorphine•1*IPA (Form A) (A526-010-A1) 2 - Apomorphine free base (A530-020-01) 3 - Apomorphine•1.0 TBME (A505-080-D1)

After single forms or mixtures of single forms were suspended in anhydrous isopropanol at 45-50° C. and stirred for up to 6 days, the following observations were made:
Single form apomorphine•1*IPA (Form A) was unchanged by XRPD and ¹H NMR.
A mixture of approximately equimolar amounts of apomorphine•1*IPA (Form A) and solvent free amorphous apomorphine free base generated a product that was consistent with apomorphine•1*IPA (Form A). The product appeared to consist of predominantly crystalline material by XRPD and contained a stoichiometric amount of IPA (18.3% w/w by ¹H NMR), indicating that the treatment promoted conversion of the amorphous material to crystalline IPA solvate.
Apomorphine•1.0*TBME (generated by suspension equilibration of apomorphine•1*IPA in TBME) when stirred in the presence of apomorphine•1*IPA (Form A) reverted back into apomorphine•1*IPA (by XRPD, see FIG. 21, and ¹H NMR analyses).
A composite of the three forms, when stirred under these conditions generated apomorphine•1*IPA (Form A), (by XRPD and ¹H NMR analyses).

These findings suggest that if other forms are generated in situ they can be readily controlled by suspension equilibration treatment with IPA.

### Equilibrium Desiccator Analysis

Experiments were performed on samples of apomorphine•1*IPA (demonstration batch A0526-004-B1, after single crystallization), apomorphine•1*IPA (demonstration batch A0526-010-A1, after recrystallization) an alternative solvate apomorphine•0.5*EtOH (A0505-080-G1) and amorphous apomorphine, prepared via the aqueous route (A0530-020-01). All samples were maintained under a single humidity condition of 75 to 80% RH at 18-23° C. for 144 h. Uppermost areas of exposure of the samples were the same for all solids and the results are summarized in Table 15.

Water uptake of the apomorphine solvates was insignificant after 144 h, whilst the weight increase of amorphous apomorphine (A0486-080-C1) was 2.0% w/w after 1 h and had stabilized at this level after completion of the investigation. Uniform equilibration was assumed and all specimens were mobile after this time and appeared physically unaltered when observed. Their compositions by ¹H NMR and form by XRPD were unchanged.

TABLE 15
Equilibrium desiccator analysis, performed at 75-80% RH at 18-23° C.

| | | Weight changes | | | | | | | |
|---|---|---|---|---|---|---|---|---|---|
| Reference | Provenances | 0 h | 1 h | 3 h | 20 h | 24 h | 48 h | 72 h | 144 h |
| A0505-110-A1 | Apomorphine•1*IPA | 6645.40 | 6645.30 | 6645.47 | 6645.25 | 6645.35 | 6645.28 | 6645.36 | 6645.25 |
| (input | (recrystallized from | | | | | | | | |
| A0526-110-A1) | IPA) | | | | | | | | |
| Δwt. | | 88.62 | 88.52 | 88.69 | 88.47 | 88.57 | 88.50 | 88.58 | 88.47 |
| Δwt. percent | | 0.0% | −0.1% | 0.1% | −0.2% | −0.1% | −0.1% | 0.0% | −0.2% |
| A0505-110-B1 | Apomorphine•1*IPA | 6752.15 | 6751.98 | 6752.05 | 6751.96 | 6752.09 | 6751.92 | 6751.92 | 6752.00 |
| (input | (crystallized from IPA | | | | | | | | |
| A0526-004-B1) | reaction mixture) | | | | | | | | |
| Δwt. | | 73.99 | 73.82 | 73.89 | 73.80 | 73.93 | 73.76 | 73.76 | 73.84 |
| Δwt. percent | | 0.0% | −0.2% | −0.1% | −0.3% | −0.1% | −0.3% | −0.3% | −0.2% |
| A0505-110-C1 | Apomorphine•0.5* | 6788.79 | 6788.88 | 6788.83 | 6788.74 | 6788.81 | 6788.84 | 6789.09 | 6788.90 |
| (input | EtOH solvate | | | | | | | | |
| A0505-080-G1) | (from suspension | | | | | | | | |
| Δwt. | equilibration in | 33.91 | 34.00 | 33.95 | 33.86 | 33.93 | 33.96 | 34.21 | 34.02 |
| Δwt. percent | ethanol) | 0.0% | 0.3% | 0.1% | −0.1% | 0.1% | 0.1% | 0.9% | 0.3% |
| A0505-110-D1 | Aqueous procedure | 6818.45 | 6819.94 | 6819.01 | 6819.72 | 6819.82 | 6819.57 | 6819.99 | 6819.86 |
| (input | | | | | | | | | |
| A0530-020-01) | | | | | | | | | |
| Δwt. | | 72.72 | 74.21 | 73.28 | 73.99 | 74.09 | 73.84 | 74.26 | 74.13 |
| Δwt. percent | | 0.0% | 2.0% | 0.8% | 1.7% | 1.9% | 1.5% | 2.1% | 1.9% |

TABLE 15
Equilibrium desiccator analysis, performed at 75-80% RH at 18-23° C.
(continued)

| | | Input | | | | |
|---|---|---|---|---|---|---|
| | | | Solvent | | Output | |
| | | | contents | | | Solvent |
| | | Forms | (¹H NMR, | | Forms | contents (¹H |
| Reference | Provenances | (XRPD) | w/w) | DSC (onsets) | (XRPD) | NMR, w/w) |
| A0505-110-A1 | Apomorphine•1*IPA | Form A, | 18.9% | 110° C., 204° C. | Form A, | 18.6% |
| (input | (recrystallized from | crystalline | | | crystalline | |
| A0526-110-A1) | IPA) | | | | | |
| A0505-110-B1 | Apomorphine•1*IPA | Form A, | 19.2% | Not obtained | Form A, | 18.7% |
| (input | (crystallized from | crystalline | | | crystalline | |
| A0526-004-B1) | IPA reaction | | | | | |
| | mixture) | | | | | |
| A0505-110-C1 | Apomorphine•0.5* | Crystalline | 7.5% | 135° C., 155° C. | Crystalline | 7.7% |
| (input | EtOH solvate | | | 146° C., 155° C. | | |
| A0505-080-G1) | (from suspension | | | 160° C., 206° C. | | |
| | equilibration in | | | | | |
| | ethanol) | | | | | |
| A0505-110-D1 | Aqueous procedure | Amorphous | N/A | 94° C. & | Amorphous | N/A |
| (input | | | | 164° C. | | |
| A0530-020-01) | | | | | | |

*N/A—not applicable.

### Gravimetric Vapour Sorption Analyses

Apomorphine•1*IPA Form A (A0526-010-A1) was subjected to a step profile of 0 to 90% RH in 10% RH increments followed by a desorption profile of 85% RH to 0% RH in 10% RH decrements and the temperature was maintained at 25±0.1° C. The weight changes during the sorption/desorption cycle were monitored.

From 0% to 90% RH a weight change of <0.1% w/w was observed attributed to absorption of surface water, not bonded, this water was lost in desorption. The absorption profile showed that the sample was not hygroscopic according to European Pharmacopoeia classification of equilibration of the API (Table 16). Form changes were not observed.

TABLE 16
Hygroscopicity classifications (European Pharmacopoeia)

| | Wt. increases at 80% RH (approximately 25° C.) |
|---|---|
| Classification | after 24 h |
| Non hygroscopic | <0.2% |
| Slightly hygroscopic | ≧0.2% and <2% |
| Hygroscopic | ≧2% and <15% |
| Very hygroscopic | ≧15% |
| Deliquescent | sufficient water is absorbed to form a liquid |

### Example 12: Solvent and Polymorph

### Solvent Screen

The following solvates were identified: formamide, acetone, TBME, methyl acetate, THF, ethanol, acetonitrile, 2-propanol (solvate investigated in the polymorph screen), water, 1,4-dioxane, nitromethane, pyridine, and ethylene glycol. The solvates contained variable amounts of amorphous apomorphine free base. The diffraction patterns and proposed onset temperatures of solvent release for each of the solvates were measured and are reported in Example 14 and the corresponding figures.

Five crystalline solvates were unintentionally generated during the polymorph screen. Characterization data for these appears in Example 14 and the corresponding figures.

### Polymorph Screen

Apomorphine•1*IPA Form A is advantageous because it is easily prepared and the physical form is well controlled by crystallization. No alternative apomorphine•1*IPA polymorphic forms were identified during the polymorph screen and the solvate exhibited good resilience to elevated humidity conditions.

Some solvent exchange of IPA occurred when other solvents were present at high concentrations, but this can be avoided during the final recrystallization and isolation from IPA by employing IPA of sufficiently high grade (e.g., INEOS) pharm grade with a purity of 99.96% w/w and containing a low level of water (0.1% w/w).

### Example 13: Experimental Methods and Optimized Crystallization Procedures

### Instrumentation

DSC.

A Mettler Toledo DSC 821 instrument was used for the thermal analysis operating with STARe™ software. The analysis was conducted in 40 μl open aluminium pans, under nitrogen and sample sizes ranged from 1 to 10 mg. Typical analysis method was 20 to 350 at 10° C./minute.

FTIR.

FTIR Spectra were acquired using a PerkinElmer Spectrum One FTIR spectrometer. Samples were analyzed directly using a universal ATR attachment in the frequency range 4000 to 600 cm⁻¹. Spectra were processed using Spectrum CFD, vs. 4.0 PerkinElmer Instruments LLC.

GVS.

The sample (approximately 7 mg) was placed into a wire mesh vapour sorption balance pan and loaded inside a Hiden Analytical Instruments IGAsorp vapour sorption balance and maintained at 25±0.1° C. The sample was then subjected to s step profile from 0 to 90% RH in 10% increments and then a desorption profile from 85% to 0% RH in 10% decrements. The weight change during the sorption cycle was monitored, allowing the hygroscopic nature of the sample to be determined.

¹H NMR.

¹H NMR Spectra were acquired using a Bruker 400 MHz spectrometer and data was processed using Topspin. ¹H NMR Samples were prepared in DMSO-d₆ and referenced to the non-deuterated solvent residual at 2.50 ppm.

### ¹H NMR Assay

### Performed on Apomorphine•1*IPA Form A (A0526-010-A1)

Internal standard TCNB (30.8 mg, F.W. 260.89, 99%) and apomorphine (A0526-010-A1) (30.3 mg, F.W. 267.33) were dissolved in CD₃OD (3.0 ml) and the ¹H NMR spectrum was acquired using an extended relaxation (20 s) method (FIG. 23).

Signal at δ=8.4 ppm (1H, d) corresponded to an aryl signal associated with the product (∫1.00). Signal at δ=8.5 ppm (s) corresponded to the internal standard TCNB (∫1.27).
Apomorphine free base: 81.4% w/w
IPA content=19.2% w/w
Water content (KF)=<0.1% w/w
Apomorphine free base (on anhydrous solvent free basis):
81.4%*100/(100%-19.2%-0.1%)=100.9% w/w (on anhydrous solvent free basis)
Apomorphine to IPA solvent equivalents: ∫1.00 (1H, δ=8.3 ppm) to ∫6.3 (6H, δ=1.2 ppm); equates to 1.0 apomorphine to 1.1 IPA.

### HPLC Method

METCR2498

Column: Hypersil BDS C18, 150×4.6 mm, 5 μm

Inj. volume: 10 μl

Detection: Ultra violet @ 280 nm

Mobile phase A: Sodium octanesulfonate, pH2.2

Mobile phase B: Acetonitrile

Gradient:

| Time (mins) | % A | % B |
|---|---|---|
| 0 | 85 | 15 |
| 2 | 85 | 15 |
| 32 | 68 | 32 |
| 37 | 68 | 32 |
| 37.1 | 85 | 15 |
| 50 | 85 | 15 |

Flow rate: 1.5 ml/min

Column temperature: 35° C.

Run time: 50 minutes

Integration time: 37 minutes

Wash vial: Water/acetonitrile, 1/1 v/v

To prepare 2 L mobile phase A, weigh 2.2 g of sodium octanesulfonate monohydrate into a 2 L duran and dissolve in 2000 ml of deionized water. Adjust to pH 2.2 (±0.1) using a dilute orthophosphoric acid solution (1:1 with deionised water), mix well and degas by sonication.

To prepare 1 L mobile phase B, transfer 1000 ml acetonitrile to a 1 L mobile phase reservoir and degas by sonication.

To prepare 500 ml sample diluent transfer 500 ml deionized water to a 500 ml mobile phase duran. Add 5 ml of acetic acid. Mix well and degas by sonication.

Different volumes of mobile phase and/or sample diluent may be prepared as long as the proportions of each component remain the same.

### LC-MS

Routine LC-MS data were collected using a Micro Mass platform LCZ interfaced with: CTC Analytics liquid sample changer system, Waters 2487 dual λ absorbance detector and Agilent series 1100 binary pump.

The instrument used a ZMD quadrupole mass analyser based detector and the mass separated ions were detected via a photomultiplier system. The ZMD quadrupole instrument was calibrated up to 2000 Da.

### PLM

The instrument used for digital capture was an Olympus BX41 microscope with digital camera attachment. The magnification was ×100 and ×400. Samples were observed under plane-polarized and cross-polarized light.

TGA:A Perkin Elmer Pyris Diamond TG/DTA 6300 was used to measure the weight loss as a function of temperature from 30 to 600° C. The scan rate was 10° C./min and the purge gas was nitrogen.

HSM: The instrument used for digital capture was an Olympus BX41 microscope with digital camera and hot stage attachment. The magnification was ×100 and ×400. Samples were observed under plane-polarized and cross-polarized light.

XRPD analysis was carried out using a Bruker D2 Phaser powder diffractometer equipped with a LynxEye detector. The specimens underwent minimum preparation but, if necessary they were lightly milled in a pestle and mortar before acquisition. The specimens were located at the center of a silicon sample holder within a 5 mm pocket (approximately 5-10 mg).

The samples were continuously spun during data collection and scanned using a step size of 0.02° two theta (20) between the range of 4° to 40° two theta. Data was acquired using either 3 minute or 20 minute acquisition methods. Data was processed using Bruker Diffrac.Suite.

### Preparative Methods

### Apomorphine•1*IPA Form a by Non-Aqueous Procedure

### Procedure

Scheme 7 summarizes the steps performed in the optimized synthesis. Each step is described in more detail below:
1. Charge apomorphine hydrochloride (1.0 wt), ascorbic acid-6-palmitate (0.01 wt), IPA (30 vol) and stir at 18-23° C.
2. Vacuum/nitrogen purge the vessel ×3 at 18 to 23° C. and place under a flow of nitrogen.
3. Heat the slurry to 60-65° C.
4. Charge 3-amino-1-propanol (1.1 eq, 0.25 vol) maintaining 60-70° C. (target 60-65° C.).
5. As required cool the reaction mixture to 60-65° C. Note: The reaction mixture has been held at this point for 2 h at reflux (83° C.) with no significant change in purity profile (A0513-98).
6. Stir for 15-20 minutes and check for full dissolution.
7. On full dissolution, clarify the reaction mixture through a ≦1 μm filter and line rinse with IPA (3 vol) at 60-65° C. Note: the reaction solution is highly unstable to air whilst in hot solution; therefore total exclusion of air is required.
8. Concentrate the reaction mixture under reduced pressure to approximately 10 vol maintaining 55-65° C. Note: The distillation has been performed over 6 h at 60-65° C. with no significant change in purity profile of the material isolated. Minor discoloration of the slurry was observed; pale yellow (A0513-126).
9. Heat the slurry to reflux (expect 82° C.) and check for full dissolution. Note: The reaction mixture has been held at this point for 2 h at reflux (83° C.) with no significant change in purity profile (A0513-98).
10. Cool the slurry to 68-71° C. and age for 1 h (crystallization is expected during the cool down period approximately 72° C.).
11. Slow the stirring for the remainder of the preparation to the minimum effective rate and check for crystallization: if crystallization has not occurred seed the reaction with 0.1 wt % of seeds and age for 1 h.
12. Cool the slurry to 18-23° C. over 5-6 h at an approximately constant rate.
13. Age the slurry at 18-23° C. for 2-16 h.
14. Filter the solid and wash with IPA (2×3 vol) at 18-23° C. under nitrogen.
15. Dry the solid under a flow of nitrogen for at least 30 min.
Expected output: 88 to 94% (theoretical).

### Demonstration Batch (A0526-004)

Apomorphine hydrochloride (650 g, JM 11-00645), ascorbic acid-6-palmitate (6.50 g, 0.01 wt, 1.0% w/w) and IPA 19.5 L (30.0 vol) were charged to a 30 L vessel equipped with flange lid and overhead stirrer.
The flask underwent ×3 evacuations (approximately 5 minutes) and nitrogen purge cycles at 17.9° C. (final temperature due to cooling 10.2° C.).
The suspension was heated to 60° C. and 3-amino-1-propanol (176.78 g, 0.28 vol, 1.1 equiv.) was charged (end temperature recorded 68.9° C.).
After stirring the orange/red solution was clarified through an in-line 0.7 μm GFF filtration assembly at the same temperature to afford a clear red solution, a small amount of particulates were present on the surface of the filtration media.
Concentration to approximately 10.0 vol was performed under 290-300 mbar at 55-60° C.
The now dark green, concentrated suspension was heated to reflux (84° C.) to effect dissolution.
The solution was cooled to 18-23° C. over 13 h to give a green/brown suspension.
The suspension was filtered through 0.7 μmGFF/20 μmfilter cloth sandwich and pulled free of surplus solvents before applying ×2 isopropanol cake washes (2×3.0 vol).
The filter bed was de-liquored under a robust flow of nitrogen for 2.5 h and crumbled.
Drying of the powdery solid was continued using the same apparatus and conditions (72 h, 18-23° C.).

Output: 605 g, 85% th corrected for 18.9% w/w IPA assay against TCNB internal standard (A0526-004-B1, 99.4% w/w on anhydrous solvent free basis).

Photomicrograph: prisms (FIG. 26)

XRPD: sharp diffraction peaks, consistent with previous batches (FIG. 25)

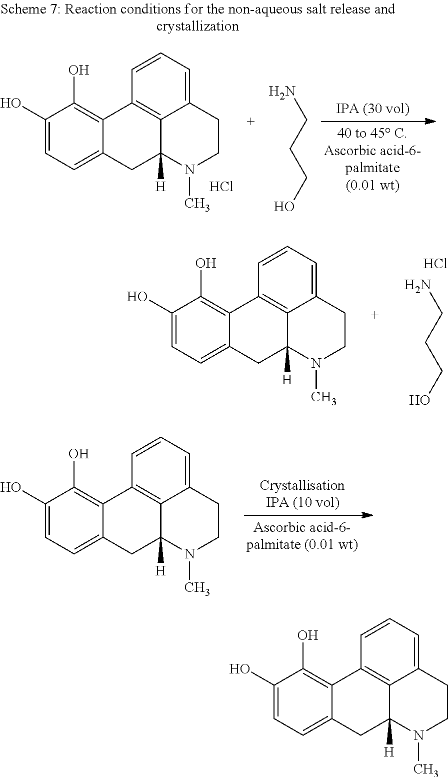

Recrystallization (A0526-010) Note: All manipulations were performed under nitrogen.
Apomorphine•1*IPA (550 g, A0526-004-B1), ascorbic acid-6-palmitate (5.50 g, 0.01 wt) and GF/F clarified IPA 6050 ml (11.0 vol) were charged to a 20 L vessel equipped with flange lid, internal lid O-ring and overhead stirrer.
The flask underwent ×3 evacuations (approximately 80 mbar, 5 minutes apiece) and nitrogen purge cycles at 24° C.
The suspension was then heated to 83° C. and full dissolution was achieved.
The solution was then cooled until crystals first appeared.
The turbid solution was aged at this temperature for 1 h and cooled to 18° C. over approximately 16 h with an agitation rate of 150-160 rpm.
The off-white suspension was filtered through 20 μm filter cloth placed above 0. μmGFF and pulled free of surplus solvents, before applying ×2 IPA cake washes (2×3.0 vol).
The filter bed was de-liquored for 2.5 h at 18-23° C., off-loaded from the filtration assembly and sieved through 1.4 mm mesh.

Output (A0526-010-A1): 503 g, 91% th (100.7% w/w on anhydrous solvent free basis, 19.2% w/w IPA, by ¹H NMR), assay against TCNB internal standard.

XRPD: sharp diffraction peaks, consistent with previous batches (FIG. 29).

QC analysis: see FIG. 30.

### Optimize Production Process

### Stage 1 (Salt Release)

| Step | Operation |
|---|---|
| 1 | Charge Vessel A with apomorphine hydrochloride (code RM0776, |
| | 0.50 wt), ascorbic acid-6-palmitate (USP) (code RM0771, 0.025 |
| | wt), propan-2-ol (IPA, pharma grade) (code RM0767, 14.0 vol, |
| | 11.0 wt) and stir at 18-23° C. |
| 2 | Perform three evacuation and argon purge cycles at 18-23° C. and |
| | place the vessel and contents under a flow of argon |
| 3 | Heat the slurry to 60-65° C. |
| 4 | Charge degassed 3-amino-1-propanol (code RM0768, 1.05 eq, |
| | 0.13 wt) maintaining 60- 65° C.*, followed by a line rinse |
| | of degassed IPA (pharma grade), (code RM0767, 1.0 vol, 0.8 wt) |
| 5 | Stir for 15-20 minutes at 60-65° C. and check for full dissolution. |
| 6 | On full dissolution, clarify the reaction mixture through a ≦1 μm |
| | filter into Vessel B and line rinse with IPA (pharma grade) (code |
| | RM0767, 1.5 vol, 1.2 wt) at 60-65° C. (Note: the reaction solution |
| | is unstable to air and therefore total exclusion of air is required). |
| 7 | Start step 0 as required and continue with step 0. |
| 8 | Rinse vessel A with 50 L of IPA (pharma grade) and discard. |
| 9 | Charge vessel A with apomorphine hydrochloride (code RM0776, |
| | 0.50 wt), ascorbic acid-6-palmitate (USP) (code RM0771, 0.025 |
| | wt), IPA (pharma grade) (code RM0767, 14.0 vol, 11.0 wt) and |
| | stir at 18-23° C. |
| 10 | Perform three evacuation and argon purge cycles at 18-23° C. and |
| | place the vessel and contents under a flow of argon |
| 11 | Heat the slurry to 60-65° C. |
| 12 | Charge degassed 3-amino-1-propanol (code RM0768, 1.5 eq, 0.13 |
| | wt) maintaining 60-65° C.*, followed by a line rinse of degassed |
| | IPA (pharma grade), (code RM0767, 1.0 vol, 0.8 wt) |
| 13 | Stir for 15-20 minutes at 60-65° C. and check for full dissolution. |
| 14 | On full dissolution, clarify the reaction mixture through a ≦1 μm |
| | filter and line rinse with IPA (pharma grade) (code RM0767, 1.5 |
| | vol, 1.2 wt) at 60-65° C. (Note: the reaction solution is unstable to |
| | air and therefore total exclusion of air is required). |
| 15 | Concentrate the combined reaction mixture in vessel B under |
| | reduced pressure to approximately 9 vol maintaining 55-65° C.**. |
| 16 | Heat the slurry to reflux at ambient pressure (expect 82° C.) and |
| | check for full dissolution. |
| 17 | Cool the slurry to 60-65° C. (target 62° C.). |
| 18 | To a separate vessel charge seeds (code NT0713 or FP0190, 0.014 |
| | wt) and IPA (pharma grade) (code RM0767, 1.0 vol, 0.8 wt) to |
| | generate a seed slurry. |
| 19 | Charge the seed slurry to the vessel at 60-65° C. (target 62° C.). |
| | Check for onset of crystallization and age for 55-65 minutes. |
| 20 | Slow the stirring for the remainder of the preparation to the |
| | minimum effective rate. |
| 21 | Cool the slurry to 18-23° C. over 4-6 h at an approximately |
| | constant rate. |
| 22 | Age the slurry at 18-23° C. for 16-20 h. |
| 23 | Filter the solid and wash with clarified, degassed IPA (pharma |
| | grade) (code RM0767, 2 × 3.0 vol, 2 × 2.4 wt) at 18 to |
| | 23° C. under nitrogen. |
| 24 | Dry the solid under a flow of nitrogen at ≦25° C. for at least 4 h |
| | until ≦20% w/w IPA (Target 18-19% w/w IPA) by ¹H NMR |
| | analysis. (MET/PR/1483). |

*Stability: The reaction mixture has been held at this point for 2 h at reflux (83° C.) with no significant change in purity profile (A0513-98) and stirred for 5 days at 65° C. with no significant change in purity (A0526-014).
**Stability: The distillation has been performed over 6 h at 60-65° C. with no significant change in purity profile of the material isolated. Minor discoloration of the slurry was observed; pale yellow (A0513-126).

### Stage 2 (Crystallization)

| Step | Operation |
|---|---|
| 1 | Charge apomorphine.IPA (crude) (code NT0713, 1.00 wt) to |
| | vessel A. |
| 2 | Dissolve ascorbic acid-6-palmitate (USP) (code RM0771, 0.01 wt), |
| | in propan-2-ol (IPA, pharma grade) (code RM0767, 11.0 vol, 8.6 |
| | wt) in Vessel B, and adjust to 18-23° C. |
| | Charge the contents of vessel B to vessel A via an in line filter. |
| 3 | Perform three evacuation and argon purge cycles at 18-23° C. and |
| | place the vessel A and contents under a flow of argon. |
| 4 | Heat the slurry to 82-85° C. |
| 5 | Stir for 15-20 minutes at 82-85° C. and check for full dissolution. |
| 6 | Cool the slurry to 65-70° C., check for onset of crystallization and |
| | age for 55-65 minutes. (Crystallization is expected at ≦70° C.) |
| 7 | Slow the stirring for the remainder of the preparation to the |
| | minimum effective rate. |
| 8 | Cool the slurry to 18-23° C. over 4-6 hours at an approximately |
| | constant rate. |
| 9 | Age the slurry at 18-23° C. for 16-20 hours. |
| 10 | Filter the solid and wash with clarified IPA (pharma grade) (code |
| | RM0767, 2 × 3.0 vol, 2 × 2.4 wt) at 18-23° C. under nitrogen. |
| 11 | Dry the solid under a flow of nitrogen at ≦25° C. for at least 4 h |
| | until ≦20% w/w IPA (Target 18-19% w/w IPA) by ¹H NMR. |
| | MET/PR/1483 |

### Amorphous Apomorphine by Aqueous Procedure

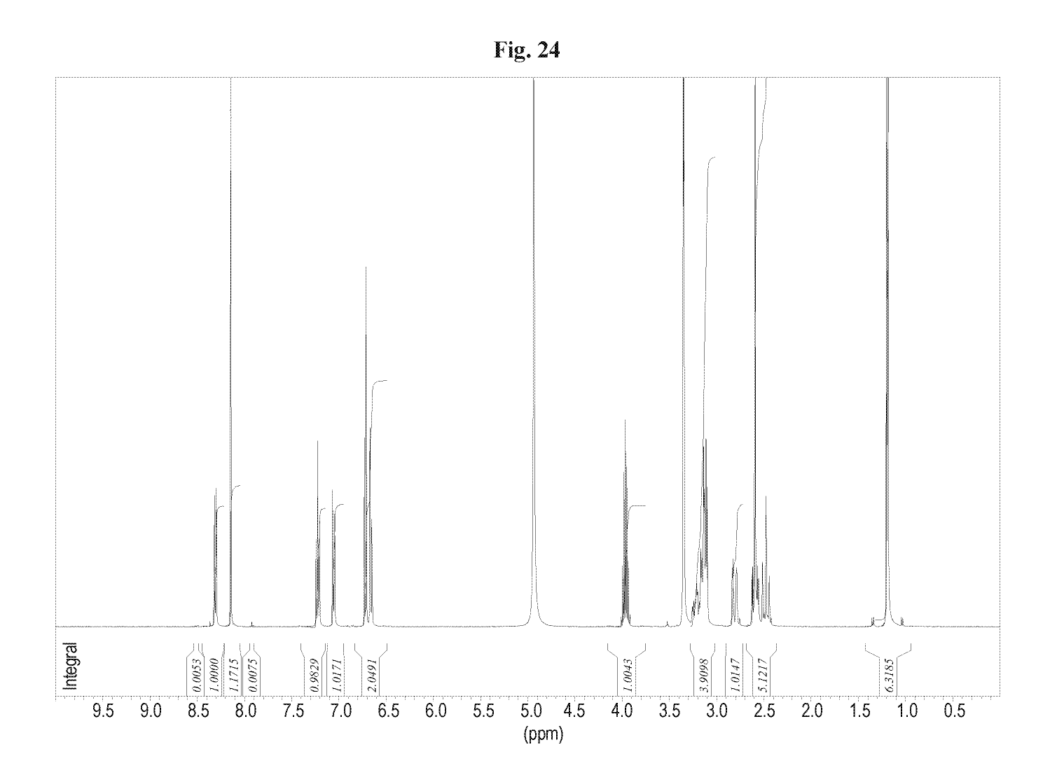

Scheme 8 summarizes the steps performed in the optimized aqueous-based procedure. Each step is described in more detail below:
1. Apomorphine hydrochloride (15.0 g, 1.0 wt) was dissolved in 0.1% w/w aqueous sodium metabisulfite solution (1005 ml, 67 vol).
2. 1N aqueous sodium bicarbonate solution was added (165 ml, 11 vol) at approximately 25° C.
3. The stirring continued for approximately 30 minutes at approximately 25° C.
4. The resulting precipitate was filtered, using Whatman paper #43 ø 110 mm (Buchner flask 2000 ml) using a vacuum pump.
5. The precipitate was washed with water (2×495 ml, 2×33 vol) and dried under N₂ flow using a vacuum pump for 4 h.
Output: 12.7 g, 97% (theoretical).
¹H NMR data is shown in FIG. 24.
XRPD data is shown in FIG. 25.

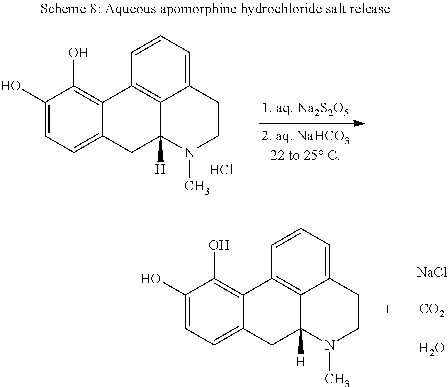

### Example 14: Apomorphine Solvates/Solvent

The following solvates were identified: formamide, acetone, TBME, methyl acetate, THF, ethanol, acetonitrile, 2-propanol (solvate investigated in the polymorph screen), water, 1,4-dioxane, nitromethane, pyridine, and ethylene glycol. The solvates contained variable amounts of amorphous apomorphine free base. The diffraction patterns and proposed onset temperatures of solvent release for each of the solvates were measured and are reported below.

### Solvates

### Apomorphine Formamide Solvate (A0530-004-F1)

FIG. 33 shows XRPD data for apomorphine formamide solvate, sample A0530-004-F1. Proposed onset temperature of solvent (formamide) release by DSC: 96.9° C.

2Theta: 7.489, 7.588, 8.192, 9.130, 10.978, 12.232, 13.529, 14.037, 14.928, 19.569, 20.241, 20.706, 21.859, 22.547, 22.898, 23.328, 24.066, 24.307, 25.313, 26.047, 26.834, 29.855, 33.007.

### Apomorphine Acetone Solvate (A0530-010-F1)

FIG. 34 shows XRPD data for apomorphine acetone solvate, sample A0530-010-F1. Proposed onset temperature of solvent (acetone) release by DSC: 86.1° C.

2Theta: 7.626, 8.780, 9.443, 10.327, 12.709, 13.053, 13.986, 14.821, 15.534, 16.590, 17.130, 17.621, 18.849, 19.610, 18.358, 20.273, 21.010, 22.729, 23.122, 23.392, 24.227, 26.806, 25.503, 28.535, 29.441, 30.622.

### Apomorphine TBME Solvate (A0530-010-G1)

FIG. 35 shows XRPD data for apomorphine TBME solvate, sample A0530-010-G1. Proposed onset temperature of solvent (TBME) release by DSC: 97.5° C. (complex thermogram).

2Theta: 8.289, 10.534, 14.010, 14.927, 18.727, 20.048, 22.104.

### Apomorphine Methyl Acetate Solvate (A0530-010-H1)

FIG. 36 shows XRPD data for apomorphine methyl acetate solvate, sample A0530-010-H1. Proposed onset temperature of solvent (methyl acetate) release by DSC: 101.7° C. (complex thermogram).

2Theta: 9.085, 10.615, 11.442, 12.771, 13.172, 13.899, 14.689, 15.291, 16.883, 18.195, 18.486, 19.028, 19.570, 20.629, 21.316, 21.647, 23.009, 24.239, 25.906, 26.858, 27.850, 29.163, 33.626, 35.225.

### Apomorphine THF Solvate (A0530-010-K1)

FIG. 37 shows XRPD data for apomorphine THF solvate, sample A0530-010-K1. Proposed onset temperature of solvent (THF) release by DSC: 91.0° C. (complex thermogram).

2Theta: 10.623, 10.905, 11.891, 12.572, 13.136, 13.840, 14.779, 16.212, 17.292, 17.808, 18.808, 19.922, 21.213, 21.847, 22.833, 24.266, 27.412, 29.007.

### Apomorphine Ethanol Solvate (A0530-010-O1)

FIG. 38 shows XRPD data for apomorphine ethanol solvate, sample A0530-010-O1. Proposed onset temperature of solvent (ethanol) release by DSC: 133.8° C.

2Theta: 10.585, 11.980, 12.768, 13.091, 14.344, 14.526, 15.596, 15.960, 17.637, 18.446, 18.708, 19.678, 20.224, 20.689, 21.497, 22.467, 24.326, 25.437, 26.387, 27.577, 28.067, 32.313, 28.850, 24.036.

### Apomorphine Acetonitrile Solvate (A0530-010-Q1)

FIG. 39 shows XRPD data for apomorphine acetonitrile solvate, sample A0530-010-Q1. Proposed onset temperature of solvent (acetonitrile) release by DSC: 123.5° C. (complex thermogram).

2Theta: 5.817, 9.377, 10.668, 11.655, 12.125, 13.161, 12.795, 14.150, 14.449, 14.671, 15.752, 17.289, 18.242, 19.259, 19.823, 20.266, 21.602, 22.852, 24.344, 25.423, 26.538, 27.547, 29.244.

### Apomorphine Hydrate (A0530-010-X1)

FIG. 40 shows XRPD data for apomorphine hydrate, sample A0530-010-X1. Proposed onset temperature of solvent (water) release by DSC: 129.8° C.

2Theta: 7.593, 7.988, 8.383, 10.306, 11.305, 11.946, 12.423, 12.997, 13.397, 14.587, 16.101, 16.614, 17.128, 17.441, 17.883, 19.152, 19.716, 20.610, 22.272, 23.872, 25.025, 26.199, 27.160, 27.887, 28.535.

### Apomorphine 1,4-Dioxane Solvate (A0530-010-Z1)

FIG. 41 shows XRPD data for apomorphine 1,4-dioxane solvate, sample A0530-010-Z1. Proposed onset temperature of solvent (1,4-dioxane) release by DSC: 128.1° C.

2Theta: 8.010, 8.779, 10.552, 11.038, 13.210, 14.057, 14.797, 14.962, 15.979, 16.816, 17.625, 18.398, 18.926, 19.478, 20.407, 21.896, 22.690, 22.996, 23.746, 24.197, 25.362, 26.374, 26.775, 27.255.

### Apomorphine Nitromethane Solvate (A0530-010-AB1)

FIG. 42 shows XRPD data for apomorphine nitromethane solvate, sample A0530-010-AB1. Proposed onset temperature of solvent (nitromethane) release by DSC: decomposition prior to solvent release.

2Theta: 9.033, 10.633, 11.565, 16.594, 20.748, 21.173, 22.987, 23.652, 24.487.

### Apomorphine Pyridine Solvate (A0530-010-AF1)

FIG. 43 shows XRPD data for apomorphine pyridine solvate, sample A0530-010-AF1. Proposed onset temperature of solvent (pyridine) release by DSC: 119.8° C. (complex thermogram).

2Theta: 11.731, 13.713, 14.859, 15.135, 18.047, 19.470, 21.359, 23.508, 24.428, 25.997, 29.428.

### Apomorphine Ethylene Glycol Solvate (A0530-010-AT1)

FIG. 44 shows XRPD data for apomorphine ethylene glycol solvate, sample A0530-010-AT1. Proposed onset temperature of solvent (ethylene glycol) release by DSC: 142.7° C. (complex thermogram).

2Theta: 8.023, 10.511, 12.063, 12.899, 14.382, 14.850, 15.487, 15.925, 17.334, 18.353, 18.623, 20.078, 20.258, 20.767, 21.277, 22.049, 22.746, 23.937, 24.409, 24.975, 25.929, 26.790, 27.540, 27.874, 28.911, 29.865.

Solvates from the Polymorph Screen

### Apomorphine•0.5*Acetone Solvate (A0505-080-A2)

FIG. 45 shows XRPD data for apomorphine•0.5*acetone solvate, sample A0505-080-A2. FIG. 46 shows corresponding DSC data. The events with onsets at 130° C. and 206° C. were attributed to acetone release and melting of the crystallized desolvated solvate.

2Theta: 8.194, 10.652, 11.468, 12.344, 14.096, 14.464, 15.493, 15.739, 16.392, 18.107, 18.404, 19.499, 20.025, 20.967, 21.502, 21.824, 22.359, 24.332, 24.766, 25.719, 26.455, 26.923, 28.444, 31.670, 32.289, 29.084.

### Apomorphine•1.0*TBME Solvate (A0505-080-D1)

FIG. 47 shows XRPD data for apomorphine•1.0*TBME solvate, sample A0505-080-D1 (prepared under anhydrous conditions). FIG. 49 shows corresponding DSC data. The events with onsets at 102° C. and 206° C. were attributed to TBME release and melting of the crystallized desolvated solvate.

2Theta: 8.282, 10.529, 13.709, 13.998, 14.892, 16.521, 17.772, 18.712, 18.953, 20.067, 20.907, 22.105, 23.789, 24.712, 25.758, 26.575, 27.700, 28.807.

### Apomorphine•1.0*TBME Solvate (A0505-090-D1)

FIG. 48 shows XRPD data for apomorphine•1.0*TBME solvate, sample A0505-090-D1 (prepared under aqueous conditions).

### Apomorphine•0.2*Cumene•0.5*IPA Solvate (A0505-080-E1)

FIG. 50 shows XRPD data for apomorphine•0.2*cumene•0.5*IPA solvate, sample A0505-080-E1 (prepared under anhydrous conditions). FIG. 52 shows corresponding DSC data. The complex event with onset at 74° C. was attributed to solvent release; no sharp event that corresponded to melting of the desolvated solvate was evident.

2Theta: 7.953, 8.440, 11.182, 12.017, 12.754, 12.917, 15.942, 16.872, 17.338, 17.815, 20.374, 21.456, 23.176, 23.721, 25.627, 27.141, 24.280.

### Apomorphine•0.2*Cumene•0.5*IPA Solvate (A0505-090-E1)

FIG. 51 shows XRPD data for apomorphine•0.2*cumene•0.5*IPA solvate, sample A0505-090-E1 (prepared under aqueous conditions).

### Apomorphine•0.5*EtOH Solvate (A0505-080-G1)

FIG. 53 shows XRPD data for apomorphine•0.5*EtOH solvate, sample A0505-080-G1 (prepared under anhydrous conditions). FIG. 55 shows corresponding DSC data. The complex bimodal event and single melt event with onsets at 135° C. and 206° C. were attributed to ethanol release and melting of the crystallized desolvated solvate.

2Theta: 7.962, 10.599, 11.952, 12.778, 14.352, 14.527, 15.608, 15.925, 17.584, 18.375, 18.693, 19.688, 20.249, 20.668, 21.480, 22.159, 22.447, 24.024, 24.365, 25.404, 25.662, 26.428, 27.535, 28.036, 28.896, 29.360, 29.860, 30.262, 31.018, 32.308.

### Apomorphine•0.5*EtOH Solvate (A0505-090-G1)

FIG. 54 shows XRPD data for apomorphine•0.5*EtOH solvate, sample A0505-090-G1 (prepared under aqueous conditions).

### Apomorphine•0.5*THF Solvate (A0505-080-O2)

FIG. 56 shows XRPD data for apomorphine•0.5*THF solvate, sample A0505-080-O2 (prepared under anhydrous conditions). FIG. 57 shows corresponding DSC data. The events with onsets at 126° C. and 206° C. were attributed to THF release and melting of the crystallized desolvated solvate.

2Theta: 8.050, 10.387, 11.260, 12.351, 14.123, 15.253, 16.126, 17.808, 19.671, 19.945, 20.544, 21.211, 21.741, 24.341, 24.871, 25.247, 26.496, 27.967.

### EQUIVALENTS

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that may vary depending upon the desired properties sought to be obtained by the present invention.

### INCORPORATION BY REFERENCE

The entire contents of all patents, published patent applications, websites, and other references cited herein are hereby expressly incorporated herein in their entireties by reference.

1. A solid crystalline form of apomorphine free base, or a hydrate, a solvate, or a co-crystal thereof.

2. The solid crystalline form of apomorphine free base, or the solvate thereof, of claim 1, wherein the solid crystalline form of apomorphine solvate comprises a solvate forming solvent.

3. The solid crystalline form of apomorphine solvate of claim 1, wherein the solvate forming solvent is selected from the group consisting of: a (C₁-C₃)alkylbenzene, a dialkylbenzene, a trialkylbenzene, pyridine, pyrrole, a (C₁-C₃)alkyl-CN, a (C₁-C₃)alkyl-NO₂, a (R)₂NC(O)H wherein R is H or a (C₁-C₆)alkyl, a (C₁-C₅)alkylC(O)O— ester, a (C₁-C₈)alkanol, a (C₂-C₈)alkyl-O—(C₁-C₈)alkyl, a (C₃-C₈)cyclic ether, a (C₃-C₇)cyclic diether, a (C₂-C₆)glycol, and a mixture thereof.

4. The solid crystalline form of claim 3, wherein the solvate forming solvent is selected from the group consisting of: a formamide, acetone, t-butyl methyl ether, tetrahydrofuran, acetonitrile, nitromethane, pyridine, ethylene glycol, cumene, MeOAc, EtOAc, isopropyl acetate, MeOH, EtOH, isopropyl alcohol (IPA), n-propanol, n-BuOH, 1,4-dioxane solvate, and a mixture thereof.

5. The solid crystalline form of claim 4, wherein the solvate forming solvent is a mixture of IPA and cumene.

6. The solid crystalline form of claim 4, comprising about 0.1 to about 1.1 mol of formamide, acetone, t-butyl methyl ether, tetrahydrofuran, acetonitrile, nitromethane, pyridine, ethylene glycol, cumene, MeOAc, EtOH, isopropyl alcohol, or 1,4-dioxane per about 1 mol of apomorphine free base.

7. The solid crystalline form of claim 1, having an X-ray powder diffraction (XRPD) pattern equivalent to that of FIG. 33, FIG. 34, FIG. 35, FIG. 36, FIG. 37, FIG. 38, FIG. 40, FIG. 41, FIG. 42, FIG. 43, FIG. 44, FIG. 45, FIG. 47, FIG. 48, FIG. 49, FIG. 50, FIG. 51, FIG. 52, FIG. 56, or FIG. 57.

8. The solid crystalline form of claim 3, wherein the solvate forming solvent is a (C₁-C₈) alkanol.

9. The solid crystalline form of claim 8, wherein the solvent is isopropyl alcohol.

10. The solid crystalline form of claim 9, wherein the isopropyl alcohol is about 15% to about 25% by weight, about 16% to about 20% by weight, about 17% to about 19% by weight, about 18% to about 19% by weight, or about 18.2% by weight of a crystal of the solid crystalline form.

11. The solid crystalline form of claim 9, comprising an isopropyl alcohol mono-solvate of apomorphine free base.

12. The solid crystalline form of claim 8, wherein a crystal of the solid crystalline form has enhanced stability against discoloration or decomposition relative to amorphous apomorphine free base.

13. The solid crystalline form of claim 9, wherein a crystal of the solid crystalline form desolvates at a temperature of about 110° C.; or wherein the crystal melts at a temperature of about 204° C.

14. The solid crystalline form of claim 8, wherein a crystal of the solid crystalline form absorbs less than 0.1% w/w water from the air when allowed to equilibrate, as measured by Gravimetric Vapour Sorption (GVS), from 0% to about 90% relative humidity, and 25±0.1° C.; or wherein the crystal contains about 0.2% w/w water or less.

15. The solid crystalline form of claim 8, having an X-ray powder diffraction (XRPD) pattern with peaks at:
(i) 8.44, 12.73, 15.84, 16.85, 17.24, 20.30, 21.37, 23.16, 23.70, 24.27, 24.82, 25.53, and 27.01 degrees 2-theta;
(ii) 8.48, 11.13, 12.88, 15.96, 16.85, 16.99, 23.69, 25.61, 30.38, and 34.35 degrees 2-theta;
(iii) 7.98, 8.49, 11.17, 12.03, 12.69, 12.88, 15.97, 16.83, 17.00, 17.36, 17.72, 20.31, 21.39, 22.43, 23.02, 23.71, 24.09, 24.85, 25.60, 27.04, 30.35, 32.18, and 34.38 degrees 2-theta;
(iv) 10.585, 11.980, 12.768, 13.091, 14.344, 14.526, 15.596, 15.960, 17.637, 18.446, 18.708, 19.678, 20.224, 20.689, 21.497, 22.467, 24.326, 25.437, 26.387, 27.577, 28.067, 32.313, 28.850, and 24.036 degrees 2-theta; or
(v) 7.962, 10.599, 11.952, 12.778, 14.352, 14.527, 15.608, 15.925, 17.584, 18.375, 18.693, 19.688, 20.249, 20.668, 21.480, 22.159, 22.447, 24.024, 24.365, 25.404, 25.662, 26.428, 27.535, 28.036, 28.896, 29.360, 29.860, 30.262, 31.018, and 32.308 degrees 2-theta;
wherein each peak value is ±0.2 degrees 2-theta.

16. The solid crystalline form of claim 8, having an XRPD pattern equivalent to that of FIG. 9, FIG. 10, FIG. 25, FIG. 29, FIG. 38, FIG. 53, or FIG. 54.

17. A liquid formulation comprising a solid crystalline form of apomorphine free base or a hydrate, a solvate, or a co-crystal thereof, dissolved in a solvent.

18. The liquid formulation of claim 17, comprising a solid crystalline form of apomorphine free base, or a solvate thereof, dissolved in a solvent.

19. The liquid formulation of claim 17, further comprising an antioxidant.

20. The liquid formulation of claim 17, further comprising a pharmaceutically acceptable carrier.

21. The liquid formulation of claim 20, wherein the formulation is suitable for subcutaneous, transdermal, intradermal, intraarterial, intramuscular, intraperitoneal, intrathecal, intrapleural, intratracheal, intravenous, intranasal, sublingual, or buccal administration.

22. A method of treating a neurological disease or disorder, a movement disease or disorder, or a condition associated therewith, in a patient in need thereof, comprising administering to the patient the liquid formulation of claim 17.

23-24. (canceled)

25. A method of producing the solid crystalline form of apomorphine free base or the solvate thereof, of claim 2, the method comprising the steps of:
(a) dissolving apomorphine hydrochloride and, optionally, an anti-oxidant, in a solvent selected from the group consisting of: a (C₁-C₃)alkylbenzene, a dialkylbenzene, a trialkylbenzene, pyridine, pyrrole, a (C₁-C₃)alkyl-CN, a (C₁-C₃)alkyl-NO₂, a (R)₂NC(O)H wherein R is H or a (C₁-C₆)alkyl, a (C₁-C₅)alkylC(O)O— ester, a (C₁-C₈)alkanol, a (C₂-C₈)alkyl-O—(C₁-C₈)alkyl, a (C₃-C₈)cyclic ether, a (C₃-C₇)cyclic diether, a (C₂-C₆)glycol, and a mixture thereof, to form a solution;
(b) contacting the solution obtained in (a) with a base in an amount sufficient to generate the apomorphine free base or the solvate thereof; and
(c) subjecting the solution to conditions that result in crystallization of the apomorphine free base or the solvate thereof, thereby producing the crystalline form of apomorphine free base or the solvate thereof.

26-33. (canceled)
